(19) United States
(12) Patent Application Publication
(10) Pub. No.: US 2026/0273029 A1
(43) Pub. Date: Sep. 17, 2026

(54) DRY EYE TREATMENT

(71) Applicant: Inoculus Ventures, LLC, Washington, NC (US)

(72) Inventor: Michael Bruce Wiggin, Raleigh, NC (US)

(21) Appl. No.: 19/679,884

(22) Filed: May 18, 2026

Related U.S. Application Data
(63) Continuation of application No. 19/086,648, filed on Sep. 22, 2023, now abandoned.
(60) Provisional application No. 63/376,788, filed on Sep. 23, 2022.

(51) Int. Cl. A61K 38/47; A61K 9/00; A61K 9/08; A61K 47/02; A61K 47/10; A61K 47/12; A61K 47/18; A61K 47/26; A61K 47/32; A61K 47/38; A61P 27/02; A61P 27/04

(52) U.S. Cl. CPC A61K 38/47; A61K 9/0048; A61K 9/08; A61K 47/02; A61K 47/10; A61K 47/12; A61K 47/183; A61K 47/186; A61K 47/26; A61K 47/32; A61K 47/38; A61P 27/02; A61P 27/04; C12Y 302/01001

(57) ABSTRACT

Ophthalmic composition for removing biofilms from the eye and for treating eye inflammation, blepharitis, and dry eye comprising α-amylase and optionally other ingredients are disclosed.

### BACKGROUND

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Blepharitis is an inflammation of the eyelids and its surrounding areas. Blepharitis usually affects both eyes, including areas along the edges of the eyelids. Blepharitis is believed to occur when tiny oil glands near the base of the eyelashes become clogged, causing irritation and redness. Several diseases and conditions can be associated with blepharitis. In addition to being uncomfortable and unsightly, blepharitis often occurs as a chronic condition that is difficult to treat, largely because current existing treatments, including artificial tears, immunosuppressive drugs (restasis), short term use of antibiotics, short term use of steroids, Lymphocyte function-associated antigen-1 (LFA-1) antagonist (xiidra), hot compresses, intense pulse light treatment, or having the meibum mechanically pressed out of the Meibomian gland do not address the root cause of the disease.

A significant gap remains in the therapeutic arsenal for treating blepharitis, largely due to the limitations of existing treatments in addressing the underlying cause. The present disclosure addresses this need first by identifying biofilm formation on the eyelid as a considerable factor in driving blepharitis prognosis. Second, the present disclosure provides compositions and methods of using the same that provide effective therapeutic treatments for blepharitis and dry eye.

### SUMMARY

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

In some aspects the disclosure provides, a method for treating one or more of blepharitis and dry eye comprising administering to a subject a therapeutically-effective amount of a composition comprising an active α-amylase enzyme, or a functional fragment thereof, optionally stabilized with a concentration of CaCl₂ and one or more pharmaceutically-acceptable excipients. In some instances, the administration promotes removal of a biofilm in the eye of the subject or in the areas surrounding the eye of the subject. In some instances, the therapeutically-effective amount of the composition is administered daily for a period of at least 1 second, at least 5 seconds, at least 1 day, 1-week, or for a period of at least 1-month.

In some aspects, the disclosure provides an ophthalmic composition comprising a therapeutically-effective amount of a functionally active α-amylase enzyme for treating a blepharitis or a dry eye condition, a concentration of CaCl₂ for stabilizing the α-amylase enzyme, and one or more pharmaceutically-acceptable excipients.

In some aspects, the disclosure provides compositions and methods that comprise one an α-amylase enzyme. In some aspects the disclosure provides a method for treating one or more of blepharitis and dry eye comprising administering to a subject a therapeutically-effective amount of a composition comprising an active α-amylase enzyme, or a functional fragment thereof, stabilized with a concentration of CaCl₂ and one or more pharmaceutically-acceptable excipients. In some formulations the composition is in a wipe, e.g., a premoistened wipe or a wipe that comprises dry ingredients. In many instances, the composition is applied to the eyelid or used as a facial wipe. In some instances, the administration promotes removal of a biofilm in the eye of the subject or in the areas surrounding the eye of the subject. The composition can be administered daily, e.g., for a period long enough to provide contact with the biofilm, at least 1 second, at least 5 seconds, at least 10 seconds. The composition can be administered daily for a period of at least 1-week. In some aspects, the α-amylase enzyme shares at least 90% sequence homology, at least 95% sequence homology, or at least 99% sequence homology with an α-amylase peptide sequence from Aspergillus oryzae. In some aspects, the composition comprises between 1 mg/mL (w/v) of the active α-amylase enzyme to 20 mg/mL (w/v) of the active a-amylase enzyme, between 1 mg/mL (w/v) of the active α-amylase enzyme to 10 mg/mL (w/v) of the active α-amylase enzyme, or another suitable amount. In some aspects, the therapeutically-effective amount comprises one or more drops of a formulation having between 1 to 3000 I.U./mg of the active α-amylase enzyme. In some aspects, the concentration of the CaCl₂ ranges from 0.2% to 0.8% (w/v). In some aspects, the composition further comprises a citrate buffer.

In some aspects the disclosure provides an ophthalmic composition comprising a therapeutically-effective amount of a functionally active α-amylase enzyme for treating a blepharitis or a dry eye condition, a concentration of CaCl₂ for stabilizing the α-amylase enzyme and one or more pharmaceutically-acceptable excipients. The α-amylase enzyme in the composition can share at least 90%, at least 95%, or at least 99% sequence homology with an α-Amylase peptide sequence from Aspergillus oryzae. The composition can comprise between 1 mg/ml (w/v) of the functionally active α-amylase enzyme to 20 mg/ml (w/v) of the functionally active α-amylase enzyme. The composition can comprise between 1 to 3000 I.U./mg of the active α-amylase enzyme. In some aspects, the concentration of the CaCl₂ ranges from 0.2% to 0.8% (w/v). The composition can further comprises one or more of an ophthalmic astringent, an ophthalmic demulcent, an ophthalmic emollient, an ophthalmic hypertonicity agent, or an ophthalmic vasoconstrictor. In some formulations the demulcent can be carboxymethylcellulose sodium (CMC), e.g., from 0.2% to 3.5% of the CMC. In some formulations the demulcent can be polyvinyl alcohol (PVA), e.g., 0.1% to 5% of the PVA. In some aspects, the composition further comprises a citrate buffer.

In some formulations the composition further comprises between 0.2% (v/v) to 3.5% (v/v) of hydroxyethyl cellulose, between 0.2% (v/v) to 3.5% (v/v) of hypromellose, between 0.2% (v/v) to 3.5% (v/v) of methylcellulose, between 0.01% (v/v) to 1.0% (v/v) of dextran, between 0.001% (v/v) to 0.1% (v/v) of gelatin, between 0.01% (v/v) to 1.5% (v/v) of glycerin, between 0.05% (v/v) to 1.5% (v/v) of polyethylene glycol 300, between 0.05% (v/v) to 1.5% (v/v) of polyethylene glycol 400, between 0.2% (v/v) to 1.0% (v/v) of polysorbate, between 0.2% (v/v) to 2.0% (v/v) of propylene glycol, between 0.2% (v/v) to 6.0% (v/v) of polyvinyl alcohol, between 0.1% (v/v) to 4.0% (v/v) of povidone, between 0.001% (v/v) to 0.1% (v/v) of benzalkonium chloride (BAK), and/or between 0.0001% (v/v) to 0.01% (v/v) of polyquad (polyquaternium-1). In some formulations the composition further comprises a stabilized oxychloro complex, a sodium perborate, a Edetate disodium and Sorbic acid, a Borate, sorbitol, propylene glycol and zinc Ionic buffer, a polyhexanide (polyhexamethylene biguanide). In preferred cases, the composition may be stabilized in a citrate buffer.

The composition can be an ophthalmic composition, i.e., a composition wherein the administration is ophthalmic administration. In some aspects, the administration is topical administration to the external eye. In some aspects, the composition is formulated as a liquid. In others, the composition is formulated as a gel. Yet, in certain preferred instances the composition is formulated in a wipe, either in a dry powder format or in a moist wipe.

In some aspects, the disclosure provides a composition comprising a concentration of a functionally active α-amylase enzyme ranging from 1 mg/mL to 20 mg/mL, a concentration of CaCl₂ ranging from 0.4 to 0.8% (w/v) for stabilizing the α-amylase enzyme, a concentration of polyvinyl alcohol (PVA) ranging from 0.1% to 5%, and at least one pharmaceutically-acceptable excipient(s). In some aspects, the concentration of the functionally active α-amylase enzyme is 10 mg/mL, and/or a concentration of the polyvinyl alcohol (PVA) is 4%, and/or a concentration of the CaCl₂ is 0.4%.

In some aspects, the disclosure provides a wipe comprising a concentration of a functionally active α-amylase enzyme ranging from 1 mg/mL to 20 mg/mL, a concentration of CaCl₂ ranging from 0.4 to 0.8% (w/v) for stabilizing the α-amylase enzyme, a concentration of polyvinyl alcohol (PVA) ranging from 0.1% to 5%, and at least one pharmaceutically-acceptable excipient(s). In some formulations the wipe comprises a concentration of a functionally active a-amylase of about 10 mg/mL, a concentration of CaCl₂ of about 0.4% (w/v), and a concentration of polyvinyl alcohol (PVA) of about 4% (w/v). In some aspects, the wipe is individually wrapped.

These aspects and other features and advantages of the invention are described below in more detail.

### BRIEF DESCRIPTION OF THE FIGURES

The patent or application file contains at least one drawing executed in color. Copies of this patent or patent application publication with color drawings(s) will be provided to the Office upon request and payment of the necessary fee.

The foregoing and other features and advantages of the present invention will be more fully understood from the following detailed description of illustrative embodiments taken in conjunction with the accompanying drawings in which:

FIGS. 1A and 1B (FIG. 1A and FIG. 1B) are photographs depicting two different areas of an eye of a subject afflicted with blocked meibomian glands, shiny biofilm at the base of eyelashes, shine biofilm on eyelashes, and lash growth misdirection. After the photos were taken, the subject was treated with Blephex and Intense Pulse Light Therapy for biofilm removal treating inflammation.

FIGS. 2A and 2B (FIG. 2A and FIG. 2B) are photographs depicting the two different areas of the eye of the subject approximately 6 months after the photographs of FIG. 1A and FIG. 1B were taken. After the photos were taken, the subject received a 1-time treatment of Blephex and Intense Pulse Light Therapy for biofilm removal treating inflammation. Subsequently, subject was prescribed daily use of Avenova (0.01% hypochlorous acid (HOCL)).

FIGS. 3A and 3B (FIG. 3A and FIG. 3B) are photographs depicting the two different areas of the eye of the subject approximately 6 months after the photographs of FIG. 2A and FIG. 2B were taken. After the photos were taken, the subject received a 1-time treatment of Blephex and Intense Pulse Light Therapy for biofilm removal treating inflammation. Subsequently, subject was prescribed daily use of Avenova (0.01% hypochlorous acid (HOCL)).

FIGS. 4A and 4B (FIG. 4A and FIG. 4B) are photographs depicting the two different areas of the eye of the subject approximately 4 months after the photographs of FIG. 3A and FIG. 3B were taken. After the photos were taken, the subject received a 1-time treatment of Blephex and Intense Pulse Light Therapy for biofilm removal treating inflammation. Subsequently, subject continued to be treated with daily use of Avenova (0.01% hypochlorous acid (HOCL)).

FIGS. 5A and 5B (FIG. 5A and FIG. 5B) are photographs depicting the two different areas of the eye of the subject approximately 1-month after the photographs of FIG. 4A and FIG. 4B were taken and after the subject received a few applications of a composition comprising 1 mg/ml of α-amylase from Aspergillus oryzae and 0.01% hypochlorous acid.

FIGS. 6A and 6B (FIG. 6A and FIG. 6B) are photographs depicting the two different areas of the eye of the subject approximately 6 months after the photographs of FIG. 5A and FIG. 5B were taken. After the photos of FIG. 5A and FIG. 5B were taken, the subject received routine daily use of the composition comprising 1 mg/ml of α-amylase from Aspergillus oryzae and 0.01% hypochlorous acid. As shown in FIGS. 6A and 6B, the biofilm is visibly eliminated, eyelashes are clean of all biofilm, inflammation and redness in eye lid reduced, patient reports major improvements in dry eye symptoms.

FIGS. 7A and 7B (FIG. 7A and FIG. 7B) are photographs depicting the two different areas of the eye of the subject approximately 6 months after the photographs of FIG. 5A and FIG. 5B were taken and after routine daily use of a composition comprising 1 mg/ml of α-amylase from Aspergillus oryzae and 0.01% hypochlorous acid over at time course of approximately 6 months.

FIGS. 8A and 8B (FIG. 8A and FIG. 8B) are photographs depicting the two different areas of the eye of the subject approximately 4 months after the photographs of FIG. 6A and FIG. 6B were taken and after routine daily use of a composition comprising 1 mg/ml of α-amylase from Aspergillus oryzae and 0.01% hypochlorous acid.

FIGS. 9A and 9B (FIG. 9A and FIG. 9B) are photographs depicting an area(s) of the eye of a subject after receiving a dose of 10 mg/ml of α-amylase in 0.01% hypochlorous acid.

FIGS. 10A and 10B (FIG. 10A and FIG. 10B) are photographs depicting a direct comparison of areas of the eye before and after treatment with a composition that comprises 10 mg/ml of α-amylase in 0.01% hypochlorous acid and Ca²⁺.

FIG. 11A (FIG. 11A) is a chart illustrating the results of testing various α-amylase compositions comprising calcium cofactor (CaCl₂) in various concentrations.

FIG. 11B (FIG. 11B) is a chart quantifying the percent of biofilm removed in 10 min by various compositions comprising ranges of CaCl₂.

FIG. 12A (FIG. 12A) is a chart quantifying the results of an analysis of the effect of various preservatives, namely BAK (benzalkonium chloride), oxychloro, 0.1%, polyquad (polyquaternium-1) 0.001%, and bleach controls on enzyme stability.

FIG. 12B (FIG. 12B) is a chart quantifying the results of an analysis of the effect of various preservatives, namely BAK (benzalkonium chloride) and GenAqua/Dequest (sodium perborate) on enzyme stability.

FIG. 13 (FIG. 13) is a chart quantifying the results of an analysis of a 0.01% hypochlorous acid preservative on enzyme activity.

FIG. 14A (FIG. 14A) is a chart illustrating the results of an analysis of various demulcents, including polyethylene glycol 400 (PEG 400) 1%, carboxymethylcellulose sodium (CMC) 2.5%, glycerin 1%, povidone (PVP) 2%, propylene glycol 1%, polyvinyl alcohol (PVA) 4%, dextran 70 0.1 percent+hypromellose 0.3%.

FIG. 14B (FIG. 14B) is a chart illustrating the results of an analysis of various demulcents, including polyvinyl alcohol (PVA), PVA+α-amylase, carboxymethylcellulose sodium (CMC), and carboxymethylcellulose sodium (CMC)+α-amylase.

FIG. 14C (FIG. 14C) is a chart illustrating the results of testing with a-Amylase stabilized in 4% PVA after 36 days.

FIG. 15 (FIG. 15) is a chart illustrating the results of an ophthalmic antibiotic comparison for staph aureus biofilm reduction.

FIG. 16 (FIG. 16) is a chart illustrating the results of various thermal stabilizing sugars and demulcents on biofilm reduction, including trehalose and sucrose combinations with demulcents.

FIG. 17 (FIG. 17) is a chart illustrating the results of testing with various concentrations of trehalose, including 3%, 6.8%, and 8%.

FIG. 18A (FIG. 18A) is a chart illustrating the results of testing 50 mM of citrate buffer with sugar stabilizers and demulcents.

FIG. 18B (FIG. 18B) is a chart illustrating the results of testing 100 mM of citrate buffer with sugar stabilizers and demulcents.

FIG. 19 (FIG. 19) is a chart illustrating the results of testing the effects of hyaluronic acid and tea tree oil on compositions comprising α-amylase.

It should be understood that the drawings and pictures are not necessarily to scale.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### Definitions

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure, e.g., α-amylase or lysozyme catalytic domain(s) refer to the domain capable of breaking peptide bonds.

Proteins are said to have an “N-terminus” and a “C-terminus.” The term “N-terminus” relates to the start of a protein or polypeptide, terminated by an amino acid with a free amine group (—NH₂). The term “C-terminus” relates to the end of an amino acid chain (protein or polypeptide), terminated by a free carboxyl group (—COOH).

The term “biofilm” as used herein refers to an assemblage of microbial cells that is associated with a surface of an eyelid and is enclosed in a matrix generally made of polysaccharide material. The lower layers of a biofilm contain microbes that are bound together in a polysaccharide matrix with other organic components such as DNA, proteins, and inorganic materials. The upper layer is a looser amorphous layer extending into the surrounding medium. The fluid layer bordering the biofilm generally has stationary and dynamic sublayers.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

“Sequence identity” or “identity” in the context of two polynucleotides or polypeptide sequences makes reference to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “in vitro” includes artificial environments and to processes or reactions that occur within an artificial environment (e.g., a test tube). The term “in vivo” includes natural environments (e.g., a cell or organism or body) and to processes or reactions that occur within a natural environment. The term “ex vivo” includes cells that have been removed from the body of an individual and to processes or reactions that occur within such cells.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which it does not.

Designation of a range of values includes all integers within or defining the range, and all subranges defined by integers within the range.

Unless otherwise apparent from the context, the term “about” encompasses values within a standard margin of error of measurement (e.g., SEM) of a stated value.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p≤0.05.

### DETAILED DESCRIPTION

### I. Overview

All of the functionalities described in connection with one embodiment of the methods, compositions, or formulations described herein are intended to be applicable to the additional embodiments of the methods, compositions, or formulations described herein except where expressly stated or where the feature or function is incompatible with the additional embodiments. For example, where a given feature or function of component is expressly described in connection with one embodiment but not expressly mentioned in connection with an alternative embodiment, it should be understood that the feature or component may be deployed, utilized, or implemented in connection with the alternative embodiment unless the feature or component is incompatible with the alternative embodiment.

One of the main causes of dry eye disease is believed to be inflammation that occurs in the tissues that produce and support tear production. Blepharitis describes inflammation of the eyelid margin. This is normally an early indication of dry eye disease, and it is commonly observed in persons with dry eye disease. When patients seek medical attention for blepharitis it is normally because they are starting to experience dry eye disease symptoms. Current treatments for dry eye disease include artificial tears, immunosuppressive drugs (restasis), temporary use of antibiotics, temporary use of steroids, Lymphocyte function-associated antigen-1 (LFA-1) antagonist (xiidra), hot compresses, intense pulse light treatment, or having the meibum mechanically pressed out of the Meibomian gland.

A significant driver of blepharitis is believed to be bacterial congestion of the meibomian glands of the eyelid. It is common for persons with dry eye disease to have increased bacterial levels and specifically the presence of biofilms on the eyelid, eye, and surrounding tissue. The eyelid margin is ideal for biofilm formation as it provides moisture, oils, and nutrients for the bacteria to survive. Analysis of biofilms from the eyelids of blepharitis patients indicates the Staphylococcus genus is the most prevalent bacteria. Further analysis shows that 46% to 51% of patients with staphylococcal blepharitis test positive for Staphylococcus Aureus. S. Aureus is considered to be a more pathogenic species than other Staphylococcus bacteria found on the eyelid. As these bacteria grow and propagate on the eyelid they form biofilms.

The bacterial biofilms encapsulate bacteria in polysaccharide matrix which can make the biofilms more resistant to antibiotics and white blood cells. Bacteria in biofilms can also exchange genetic information to better survive in their environment. As the biofilm progresses the bacteria begin to release toxins to break down the eyelid tissue for nutrients. This leads to an inflammatory response from the body. As the bacteria are encased in the biofilm the body's immune response does little to stop the bacterial attack on the eyelid tissue. This results in inflamed tissues that interfere with, e.g., the production of tear components. Further complications arise as S. Aureus and S. Epidermidis from the biofilm release lipase into the Meibomian glands. This results in turning the clear oils of the Meibomian gland into cloudy, sludge-like oils that clog the Meibomian gland leading to Meibomian gland dysfunction (MGD) and poor quality tears. Current treatments of biofilms on the eyelid margin include mechanical scrubbing by ophthalmologists with specialized tools and at home eyelid cleansers and scrubs. The only FDA approved method for removing biofilms from the eyelid margin is using Blephex, a mechanical device designed to scrub off the biofilm, similar to removing plaque from teeth at the dentist.

Certain strains of α-amylase have shown certain in-vitro effectiveness at the inhibition and reduction of S. Aureus biofilms, including α-amylase from Aspergillus oryzae (Sigma-Aldrich, St. Louis, MO, Catalog #10065), α-amylase from Bacillus subtilis (Sigma, catalog #10070), α-amylase from human saliva (Sigma, catalog #A1031), and P-amylase from sweet potato. Notably, the existing data demonstrate that most human α-amylase(s) tested were effective in preventing biofilm growth, but not in breaking down existing biofilms.

This invention relates to a composition and methods for using the same for enzymatic breakdown of a biofilm in the eyelid by the use of the enzyme amylase, or by combined use of the enzymes amylase and lysozyme. Relevant characteristics of the enzyme amylase are described above, and the lysozyme enzyme is a natural antibiotic. Although amylase and lysozyme can be found naturally in the tears of healthy persons, the present disclosure hypothesizes that people with dry eye disease produce inadequate amounts of these enzymes for effectively preventing the formation of biofilms leading to an inflammatory cycle. Further, the disclosure demonstrates that the use of these enzymes in appropriate formulations for ophthalmic administration significantly reduced biofilm formation and bacterial buildup on the eyelids.

### II. Ophthalmic Formulations Comprising Enzymes

The disclosure contemplates classes of enzymes that can remove one or more layers of biofilm from an eye of a subject, thus treating a condition that is caused or exacerbated by the presence of the biofilm. In some aspects, the disclosure contemplates classes of enzymes that can remove a biofilm by, for example, enzymatic degradation of the polysaccharide resulting in the removal of the biofilm. In some aspects, the disclosure contemplates classes of enzymes that can lyse microbial cells by degrading cell-membrane components and destabilizing its anchoring to the solid surface. In preferred aspects, the enzyme contemplated by the disclosure is α-amylase and/or a combination of α-amylase and lysozyme.

Amylase is an important group of enzymes, which are classified into α, β, γ subtypes, isoamylase, glucoamylase, and others. Amylases can be found in both plant and microbial sources. Based on the mode of action, amylases can be classified into exo-amylases and endo-amylases. Exo-amylases hydrolyze substrates from the non-reducing ends, resulting in shorter end products, whereas endo-amylases act on internal glycosidic linkages in a random manner within starch molecules, resulting in oligosaccharides of various lengths. α and β-amylases have the potential to catalyze the hydrolysis of chitosan and reduce its molecular weight, which makes it more soluble and amenable to being formulated in liquid ophthalmic compositions. Multiple amylases present in, e.g., L. pneumophila are essential for hydrolyzing polysaccharides into glucose and in helping intracellular proliferation. Amylase also helps trigger pro-inflammatory responses, which further helps prevent bacterial replication.

α-amylase(s), which predominantly act on starch (polysaccharide) as the major substrate, consists of two glucose polymers—amylose and amylopectin. α-amylase helps in the hydrolysis of α-1,4 and α-1,6-glycosidic linkages, which results in the formation of small glucose (monosaccharides) and maltose (disaccharide). To function, α-amylase is essentially a metalloenzyme, which typically requires metals such as Ca²⁺, for maintaining the stability of the enzyme molecule. Sequence alignment studies have found that α-amylases possess four conserved regions that are also present within the R strands. The α-amylases can be found widely within plants, microorganisms, and higher animals. The metabolic products obtained by the enzymatic action of α-amylases are oligosaccharides of various lengths including, branched malto-oligosaccharides possessing 6-8 glucose units that have -1,6 and -1,4, linkages, maltose, and maltotriose. These amylase enzymes can bind with substrates via catalytic groups that catalyze breakage of the glycosidic bond.

The present disclosure contemplates compositions comprising α-amylases complexed with a suitable amount of a metal, preferentially Ca²⁺, for maintaining stability of the enzyme. An amylase of the disclosure can be, for example, prepared by recombinant technology and it can have at least 99%, at least 95%, at least 90%, at least 85%, at least 80%, at least 75%, at least 70%, at least 65%, at least 60%, at least 55%, at least 50%, at least 45%, at least 40%, at least 35%, or at least 30% homology with an α-amylase peptide sequence from Aspergillus oryzae. An α-amylase of the disclosure can be prepared by the expression of an appropriate nucleic acid molecule from a recombinant organism that can have at least 99.99%, at least 99.9%, at least 99%, at least 95%, at least 90%, at least 85%, at least 80%, at least 75%, at least 70%, at least 65%, at least 60%, at least 55%, at least 50%, at least 45%, at least 40%, at least 35%, or at least 30% homology with a nucleic acid sequence encoding an α-amylase peptide sequence from Aspergillus oryzae. An appropriate nucleic acid sequence for the preparation of an α-amylase of the invention can be a degenerate sequence. The percent homology between sequences can be calculated using a plurality of algorithms, including the algorithms described in the definition.

Non-limiting examples of amylases contemplated by the disclosure and their preliminary assessment of their activity on Biofilms is listed on Table 1:

TABLE 1

| | | | Prohibits |
|---|---|---|---|
| Research | | Reduces | Biofilm |
| Grade: | Sigma Aldrich | Biofilm | Growth |
| α-Amylase | α-Amylase from porcine | unknown | unknown |
| | pancreas | | |
| | α-Amylase from Bacillus subtilis | Only after | Yes |
| | | 3 hr | |
| | α-Amylase from Aspergillus | Yes <10 | Yes |
| | oryzae | min | |
| | α-Amylase from human saliva | No | Yes |
| | α-Amylase from Bacillus | unknown | unknown |
| | amyloliquefaciens | | |
| | α-Amylase from Bacillus | unknown | unknown |
| | licheniformis | | |
| | α-Amylase, heat-stable from | unknown | unknown |
| | Bacillus licheniformis | | |
| β-Amylase | β-Amylase from barley | | |
| | β-Amylase from sweet potato | No | Partially |
| gluco | Glucoamylase from Rhizopus sp. | unknown | unknown |
| amylase | | | |
| | Amyloglucosidase from | unknown | unknown |
| | Aspergillus niger | | |
| | Amyloglucosidase from | unknown | unknown |
| | Rhizopus sp. | | |
| | Amylase, Maltogenic from | unknown | unknown |
| | Bacillus sp. | | |
| Food Grade: | Novozymes Food and Beverage | | |
| | Products | | |
| Oat | Fungamyl ® is a blend of high- | Yes | unknown |
| Beverages | quality exo- and endo-acting | | |
| | alpha-amylases | | |
| | BAN ®is a high-quality endo | unknown | unknown |
| | alpha-amylase | | |
| | Amylase ® AG is an exo-acting | unknown | unknown |
| | glucoamylase | | |
| | Maltogenase ® is a high-quality | unknown | unknown |
| | heat-stable exo-acting | | |
| | maltogenic amylase | | |
| Juice | Amylase ® AG 300 L delivers | unknown | unknown |
| | robust starch degradation. | | |
| | Amylase ® AG XXL is a heat- | unknown | unknown |
| | stable solution for starch | | |
| | degradation that's also highly | | |
| | stable across a wide range of | | |
| | pH values. | | |
| Brewing | Termamyl ® Classic, heat stable | unknown | unknown |
| | amylase | | |

An enzyme, e.g., an α-amylase, of the disclosure can be purchased from commercial sources and combined with other components to generate an ophthalmic composition described herein. Alternatively, an enzyme of the disclosure can be prepared, for example, by peptide synthesis or expression of an appropriate nucleic acid molecule. Non limiting examples of peptide sequencing methods include: a) liquid-phase peptide synthesis; b) solid-phase peptide synthesis, with a polystyrene resin, a polyamide resin, a PEG hybrid polystyrene resin, a PEG base resin, and/or a combination of any solid phase support; and c) synthetic biology. Non-limiting examples of methods for the expression of an appropriate nucleic acid molecule include molecular cloning and recombinant DNA technologies.

### III. Ophthalmic Compositions

An ophthalmologic composition of the disclosure can be a combination of one or more enzymes described herein with other chemical components, such as metal components that stabilize the enzyme and support its activity (e.g., Ca²⁺ and α-amylase), carriers, stabilizers, diluents, dispersing agents, suspending agents, thickening agents, and/or excipients. The ophthalmologic composition facilitates administration of the α-amylase in a functional and active form to an eye of a subject. Ophthalmologic composition can be administered in therapeutically-effective amounts, e.g., as eye drops via the optic route or the topical route, (e.g., solution, gel or cream direct application to the eyelid), via an eye-wash with a solution, gel, or cream, or via a wipe (e.g., moist wipe) containing a dosage of the enzyme.

Active compounds, including, e.g., α-amylase, can be formulated into a variety of liquid and topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

Active compounds, including, e.g., α-amylase, can be formulated into a wipe, for example a single use, individually wrapped wipe or a package having a plurality of wipes therein.

In practicing the methods of treatment or use provided herein, therapeutically-effective amounts of the enzymes described herein are administered in ophthalmologic composition to a subject having a disease or condition to be treated. In some embodiments, the subject is a mammal such as a human. A therapeutically-effective amount can vary widely depending on the severity of the disease, the age and relative health of the subject, the potency of the compounds used, and other factors. In preferred instances, the disease or condition is blepharitis, dry eye, or another associated inflammatory condition.

An enzyme described herein, e.g., α-amylase, can be present in a liquid composition in a range of from about 0.1 mg/mL to about 2000 mg/mL; from about 0.1 mg/mL to about 1000 mg/mL; from about 0.1 mg/mL to about 500 mg/mL; from about 0.1 mg/mL to about 100 mg/mL; from about 0.1 mg/mL to about 50 mg/mL; from about 0.1 mg/mL to about 25 mg/mL; from about 0.1 mg/mL to about 10 mg/mL; from about 1 mg/mL to about 2000 mg/mL; from about 1 mg/mL to about 1000 mg/mL; from about 1 mg/mL to about 500 mg/mL; from about 1 mg/mL to about 100 mg/mL; from about 1 mg/mL to about 50 mg/mL; from about 1 mg/mL to about 25 mg/mL; from about 1 mg/mL to about 10 mg/mL.

An enzyme described herein can be present in a liquid composition, e.g., α-amylase, in a range of from about 5 mg/mL to about 1000 mg/mL, from about 5 mg/mL to about 500 mg/mL, from about 5 mg/mL to about 100 mg/mL, from about 5 mg/mL to about 50 mg/mL, from about 5 mg/mL to about 25 mg/mL, or from about 5 mg/mL to about 20 mg/mL. The foregoing ranges are merely suggestive. Dosages can be altered depending on a number of variables, including, for example, the activity of the enzyme used, the disease or condition to be treated, the mode of administration, the requirements of the individual subject, the severity of the disease or condition being treated, and the judgment of the practitioner.

Each enzyme described herein, e.g., α-amylase can be present in, a single vial use tube or in a wipe, for example, in powder form or in solution form, in an amount of about 0.1 mg, of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg.

Each enzyme described herein, e.g., α-amylase can be present in, a single vial use tube or in a wipe, for example, in powder form or in solution form, in an amount that provides an activity that is greater than 15.0 U/mg, greater than 16.0 U/mg, greater than 17 U/mg, greater than 18 U/mg, greater than 19 U/mg, greater than 20 U/mg, greater than 21 U/mg, greater than 22 U/mg, greater than 23 U/mg, greater than 24 U/mg, greater than 25 U/mg, greater than 26 U/mg, greater than 27 U/mg, greater than 28 U/mg, greater than 29 U/mg, greater than 30 U/mg, greater than 31 U/mg, greater than 32 U/mg, greater than 33 U/mg, greater than 34 U/mg, greater than 35 U/mg, greater than 36 U/mg, greater than 37 U/mg, greater than 38 U/mg, greater than 39, greater than 40 U/mg, greater than 41 U/mg, greater than 42 U/mg, greater than 43 U/mg, greater than 44 U/mg, greater than 45 U/mg, greater than 46 U/mg, greater than 47 U/mg, greater than 48 U/mg, greater than 49 U/mg, greater than 50 U/mg, greater than 51 U/mg, greater than 52 U/mg, greater than 53 U/mg, greater than 54 U/mg, greater than 55 U/mg, greater than 56 U/mg, greater than 57 U/mg, greater than 58 U/mg, greater than 59 U/mg, greater than 60 U/mg, greater than 61 U/mg, greater than 62 U/mg, greater than 63 U/mg, greater than 64 U/mg, greater than 65 U/mg, greater than 66 U/mg, greater than 67 U/mg, greater than 68 U/mg, greater than 69 U/mg, greater than 70 U/mg, greater than 71 U/mg, greater than 72 U/mg, greater than 73 U/mg, greater than 74 U/mg, greater than 75 U/mg, greater than 76 U/mg, greater than 77 U/mg, greater than 78 U/mg, greater than 79 U/mg, greater than 80 U/mg, greater than 81 U/mg, greater than 82 U/mg, greater than 83 U/mg, greater than 84 U/mg, greater than 85 U/mg, greater than 86 U/mg, greater than 87 U/mg, greater than 88 U/mg, greater than 89 U/mg, greater than 90 U/mg, greater than 91 U/mg, greater than 92 U/mg, greater than 93 U/mg, greater than 94 U/mg, greater than 95 U/mg, greater than 96 U/mg, greater than 97 U/mg, greater than 98 U/mg, greater than 99 U/mg, or greater than 100 U/mg defined as one unit will liberate 1 mg of maltose from starch in 3 minutes at pH 6.9 at 20° C.

Each enzyme described herein, e.g., α-amylase can be present in, a single vial use tube or single use wipe, for example, in powder form or in solution form, in an amount that provides an activity that is between than 15.0 U/mg to 40 U/mg, between than 15.0 U/mg to 50 U/mg, between than 15.0 U/mg to 60 U/mg, between than 15.0 U/mg to 70 U/mg, between than 15.0 U/mg to 80 U/mg, between than 15.0 U/mg to 90 U/mg, between than 15.0 U/mg to 100 U/mg, between than 15.0 U/mg to 110 U/mg, between than 15.0 U/mg to 120 U/mg, between than 15.0 U/mg to 130 U/mg, between than 15.0 U/mg to 140 U/mg, between than 15.0 U/mg to 150 U/mg, between than 15.0 U/mg to 160 U/mg, between than 15.0 U/mg to 170 U/mg, between than 15.0 U/mg to 180 U/mg, between than 15.0 U/mg to 190 U/mg, or between than 15.0 U/mg to 200 U/mg defined as one unit will liberate 1 mg of maltose from starch in 3 minutes at pH 6.9 at 20° C.

A cofactor described herein, e.g., CaCl₂ can be present in a concentration that ranges from 0.01% (w/v) to 1.5% (w/v), 0.1% (w/v) to 1.5% (w/v), 0.2% (w/v) to 1.5% (w/v), 0.3% (w/v) to 1.5% (w/v), 0.4% (w/v) to 1.5% (w/v), 0.5% (w/v) to 1.5% (w/v), 0.6% (w/v) to 1.5% (w/v), 0.01% (w/v) to 1.0% (w/v), 0.1% (w/v) to 1.0% (w/v), 0.2% (w/v) to 1.0% (w/v), 0.3% (w/v) to 1.0% (w/v), 0.4% (w/v) to 1.0% (w/v), 0.2% to 0.8% (w/v), 0.3% to 0.8% (w/v), 0.4% to 0.8% (w/v), or another suitable range.

Ophthalmic compositions, e.g., wipes, drops, or another suitable composition, can be formulated using one or more physiologically-acceptable carriers comprising excipients and auxiliaries, which facilitate processing of the active compounds into preparations that can be used pharmaceutically. Formulation can be modified depending upon the route of administration chosen. Ophthalmic compositions comprising compounds described herein can be manufactured in a conventional manner, for example, by means of conventional mixing, dissolving, granulating, or emulsifying. The Ophthalmic compositions can include at least one pharmaceutically acceptable carrier, diluent, or excipient and compounds described herein or pharmaceutically-acceptable salt form.

Methods for the preparation of compositions comprising the compounds described herein can include formulating the compounds (e.g., suitable concentrations of α-amylase) with one or more inert, pharmaceutically-acceptable excipients. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. A composition can be formulated in a wipe. These compositions can also contain minor amounts of nontoxic, auxiliary substances, such as wetting or emulsifying agents, pH buffering agents, and other pharmaceutically-acceptable additives. Non-limiting examples of dosage forms suitable for use in the disclosure include liquid, elixir, nanosuspension, aqueous or oily suspensions, drops, syrups, and any combination thereof.

Non-limiting examples of pharmaceutically-acceptable excipients suitable for use in the disclosure include astringents, demulcents, emollients, granulating agents, binding agents, lubricating agents, disintegrating agents, sweetening agents, glidants, anti-adherents, anti-static agents, surfactants, anti-oxidants, gums, coating agents, coloring agents, flavouring agents, plasticizers, preservatives, sugars, suspending agents, emulsifying agents, plant cellulosic material and spheronization agents, and any combination thereof.

In some instances, an ophthalmic composition of the disclosure comprising one or more of α-amylase, a lysozyme, and/or a glucanase comprises an astringent. A suitable astringent is zinc sulfate, in approximate concentrations disclosed below:

TABLE 2
Ophthalmic astringent

Zinc sulfate, 0.25 percent

### Ophthalmic Demulcents

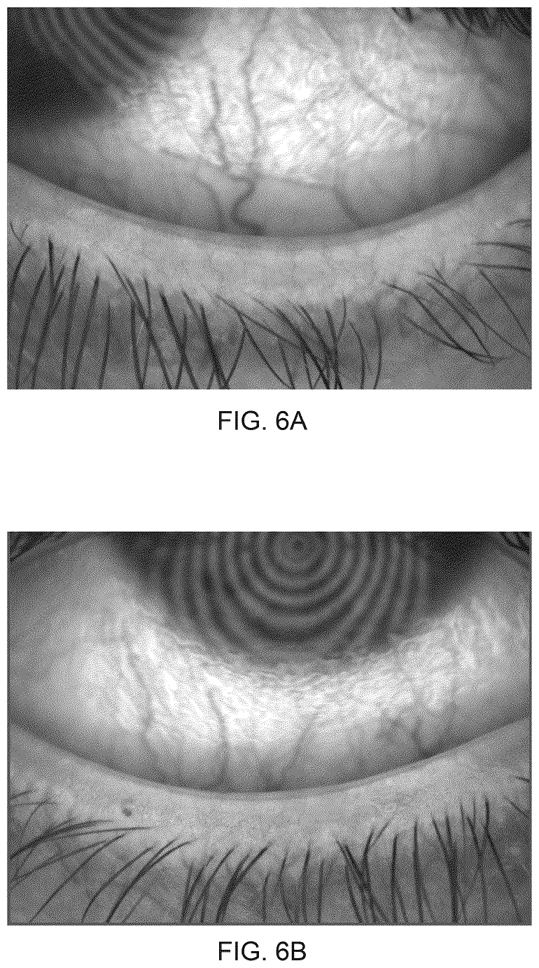

The disclosure demonstrates that α-amylase is effective at the inhibition and reduction of S. Aureus biofilms in-vivo (See, FIGS. 6A-9B). S. Aureus biofilms on the eyelid are believed to be a major cause of dry eye disease, α-amylase has not been previously utilized in the removal of biofilms from the eyelid. The disclosure contemplates ophthalmic formulations with certain ophthalmic demulcents for use in removing biofilms from the eye, eye lid, and surrounding tissues, thus treating blepharitis and dry eye conditions.

Ophthalmic demulcents can be used in eye drops to both thicken the drop, lubricate the epithelium, and relieve dryness and irritation. An ophthalmic composition of the disclosure may comprise an α-amylase(s) with one or more ophthalmic demulcents of Table 3.

TABLE 3
Ophthalmic demulcents

Carboxymethylcellulose sodium, 0.2 to 2.5 percent
Hydroxyethyl cellulose, 0.2 to 2.5 percent
Hypromellose, 0.2 to 2.5 percent
Methyl cellulose, 0.2 to 2.5 percent
Dextran 70, 0.1 percent when used with another
polymeric demulcent agent in this section
Gelatin, 0.01 percent
Glycerin, 0.2 to 1 percent
Polyethylene glycol 300, 0.2 to 1 percent
Polyethylene glycol 400, 0.2 to 1 percent
Polysorbate 80, 0.2 to 1 percent
Propylene glycol, 0.2 to 1 percent
Polyvinyl alcohol, 0.1 to 4 percent
Povidone, 0.1 to 2 percent

### Ophthalmic Emollients

Ophthalmic emollients generally refer to components that may form a film on an eye or in a skin. Generally, ophthalmic emollients can relieve dryness, itching and scaling and potentially help keep a moist layer of α-amylase on the skin or eye surface. An ophthalmic composition of the disclosure may comprise an α-amylase with one or more ophthalmic emollients of Table 4.

TABLE 4
Ophthalmic emollients

Anhydrous lanolin, 1 to 10 percent in
combination with one or more oleaginous
emollient agents included in the monograph
Lanolin, 1 to 10 percent in combination
with one or more oleaginous emollient
agents included in the monograph
Light mineral oil, up to 50 percent in
combination with one or more other
emollient agents included in the monograph
Mineral oil, up to 50 percent in combination
with one or more other emollient agents
included in the monograph
Paraffin, up to 5 percent in combination
with one or more other emollient agents
included in the monograph
Petrolatum, up to 100 percent
White ointment, up to 100 percent
White petrolatum, up to 100 percent
White wax, up to 5 percent in combination
with one or more other emollient agents
included in the monograph
Yellow wax, up to 5 percent in combination
with one or more other emollient agents
included in the monograph

### Ophthalmic Hypertonicity

Ophthalmic hypertonics are used to reduce swelling of the cornea (the front surface of your eye) caused by surgery, infection, trauma, or other eye conditions. An ophthalmic composition of the disclosure may comprise an α-amylase with one or more ophthalmic hypertonics of Table 5.

TABLE 5
Ophthalmic hypertonicity agent

Sodium chloride, 2 to 5 percent

### Ophthalmic Vasoconstrictors

The use of vasoconstrictors may help reduce inflammation on the eyelid caused by biofilms. An ophthalmic composition of the disclosure may comprise an α-amylase with one or more ophthalmic vasoconstrictors of Table 6, including: Ephedrine hydrochloride, Naphazoline hydrochloride, Phenylephrine hydrochloride, Tetrahydrozoline hydrochloride, Benzalkonium Chloride, Oxymetazoline, Brimonidine Tartrate.

TABLE 6
Ophthalmic vasoconstrictors

Ephedrine hydrochloride,
0.123 percent
Naphazoline hydrochloride,
0.01 to 0.03 percent
Phenylephrine hydrochloride,
0.08 to 0.2 percent
Tetrahydrozoline hydrochloride,
0.01 to 0.05 percent
Brimonidine Tartrate 0.025%.

An ophthalmic composition of the disclosure may comprise an α-amylase with one or more additional components of Table 7:

TABLE 7

| Ingredient | Purpose |
|---|---|
| Selenium | To treat hyperkeratinization, removal of |
| Sulfide | keratin proteins from the eye lid may help |
| | unclog meibomium glands. This drug is |
| | currently in clinical trials for this |
| | application |
| Perfluorohexyl | Reduce tear film instability |
| octane | |
| 0.01%-0.02% | Reduce Bacteria |
| Hypochlorous | |
| Acid | |
| Tea Tree Oil | Antimicrobial |
| Sucrose | Sugar Stabilizers for Amylase |
| Trehalose | Sugar Stabilizers for Amylase |
| Boric Acid | Relieves eye irritation |
| Sodium Borate | Mild Antibiotic/Buffering |
| Calcium | Ca²⁺ essential cofactor for α-amylase |
| Chloride | |
| Lecithin | Increased the lipid concentration |

An ophthalmic composition of the disclosure may comprise an α-amylase with one or more additional components of Table 8:

TABLE 8

| Ingredient | Purpose |
|---|---|
| Sorbitol | lowers the viscosity of gelling agents such as HP Guar |
| Hyaluronic | humectant that binds multiples of its weight in water |
| Acid | to lower tear osmolarity. It also adheres to the ocular |
| | surface to stabilize the thickness of the tear film |
| Sodium | Derivative of hyaluronic acid and shares many of its |
| hyaluronate | beneficial properties. Sodium hyaluronate is a |
| | humectant, lubricant, and hypo-osmotic. It is found |
| | naturally in the eye where it is produced as a response |
| | to ocular surface damage. |
| L-carnitine and | Always seen in combination as osmoprotectants. |
| Erythritol | Absorbed by dehydrated cells where they promote |
| | hydration and prevent cell shrinkage and inflammation. |
| Hydroxypropyl | A bean protein that is used as a thickening agent |
| Guar (HP Guar) | |
| Polyacrylic | It increases the viscosity and retention time of the |
| Acid | artificial tear |
| Tyloxapol | Helps to remove lipid and mucous from the surface of |
| | contact lenses |
| Tromethamine | Organic amine proton acceptor. Emulsifying agent |
| | (thins waxy agents) |

### Buffer Systems

Buffer systems are used to obtain a pH for the artificial tear that is healthy and comfortable for the eye. pH ~7 is most comfortable for dry eye patients (normal tear pH is about 6.5-7.6). Although purified human pancreatic alpha-amylase (alpha-1,4-glucan-4-glucano-hydrolase, EC 3.2.1.1) was found to be stable over a wide range of pH values (5.0 to 10.5) a buffer system may also help maintain the optimum activity of an a amylase in an ophtalmic composition of the disclosure. The disclosure contemplates that an optimal pH for the enzymatic activity of α-amylase is 5.5 and it contemplates certain compositions that use one or more of the components below to arrive at a pH between 5-7.5.

TABLE 9
Ingredient

Boric acid
Borate Buffer
Sodium-citrate
Citric Acid
Phosphate
Phosphate-acetate
Phosphate-citrate
Phosphate-citrate-
bicarbonate
Sodium hydroxide

### Electrolytes (e.g., Metal(s))

Electrolytes can be added to ophthalmic compositions to maintain or lower tear osmolarity as high osmolarity products pull water from epithelial cells, interfering with metabolism. Some of the added electrolytes are also important for corneal epithelial metabolism. Some electrolytes are part of buffer systems described above. Some electrolytes may be required as metal stabilizers of one or more enzymes in the ophthalmic compositions of the disclosure.

TABLE 10
Ingredients

| | |
|---|---|
| Calcium chloride | Ca may be needed for activation of α-amylase |

Magnesium chloride
Potassium chloride

| | |
|---|---|
| Zinc chloride | |
| Sodium chloride | |
| Sodium citrate | |
| Sodium lactate | |
| Sodium bicarbonate | |
| Calcium bicarbonate | * not in any current eye drops |

An ophthalmic composition of the disclosure may comprise one or more of a-amylase, a lysozyme, and/or a glucanase with one or more additional preservatives of Table 11:

TABLE 11
Preservatives

benzalkonium chloride0.01%
Polyquad 0.001%
GenAqua (sodium perborate)
Purite (stabilized oxychloro
complex)
Edetate disodium 0.1% and Sorbic
acid 0.1%
Polixetonium (Polyquaternium-42)
OcuPure (stabilized oxychloro
complex
Dequest (sodium perborate)
SofZia
polyhexamethylene biguanide
Ethylenediaminetetraacetic
acid EDTA)
sodium perborate

### IV. Ophthalmic Formulations

Ophthalmic compositions described herein can be formulated as eye drop containers (5 mL, 10 mL, 15 mL, 20 mL, or another suitable volume) or in unit dosage forms suitable for single administration of precise dosages. When formulated as eye drops, the formulation may be as such that each “drop” comprises a suitable amount of enzyme with a certain activity range.

In unit dosage form, the formulation is divided into unit doses containing appropriate quantities of one or more enzymes, e.g., α-amylase or α-amylase and lysozyme. The unit dosage can be in the form of a package containing discrete quantities of the formulation. Non-limiting examples are liquids in vials or ampoules. Aqueous suspension compositions can be packaged in single-dose non-reclosable containers. Multiple-dose reclosable containers can be used, for example, in combination with a preservative. Various preservatives and demulcents can be added to the composition (further described above).

Non-limiting examples of ophthalmic eye drops that can be formulated with an α-amylase, a lysozyme, or a glucanase of the disclosure include:

TABLE 12

| Product | Packaging | Active ingredients | Inactive ingredients |
|---|---|---|---|
| Advanced eye relief | 15 mL bottle | Glycerin (0.3%) | benzalkonium chloride |
| dry eye rejuvenation | | Propylene glycol | (0.01%), boric acid, |
| (Bausch & Lomb) | | (1.0%) | edetate disodium, |
| | | | potassium chloride, |
| | | | purified water, sodium |
| | | | borate, sodium chloride. |
| | | | Hydrochloric acid and/or |
| | | | sodium hydroxide may |
| | | | be used to adjust pH |
| Bion Tears(Alcon) | 28 PF vials | Dextran 70 0.1%, | Calcium chloride, |
| | (0.4 mL) | Hypromellose 2910 | magnesium chloride, |
| | | 0.3% | potassium chloride, |
| | | NOTE: Are you | purified water, sodium |
| | | looking for Tears | bicarbonate, sodium |
| | | Naturale | chloride, zinc chloride, |
| | | (discontinued)? Bion | hydrochloric acid and/or |
| | | Tears has the same | sodium hydroxide and/or |
| | | active ingredients but | carbon dioxide to adjust |
| | | some slight differences | pH. |
| | | in the inactives. | |
| BioTrue Lubricant | 10 mL PF | Glycerin 0.5% | boric acid, erythritol, |
| Eye Drops | bottle | | potassium chloride, |
| (Bausch & Lomb) | | | purified water, sodium |
| | | | borate, sodium |
| | | | hyaluronate |
| Blink Contacts | (10 mL) | (Lens wetting drops | Boric Acid; Calcium |
| Lubricating Eye | bottle | don't have their | Chloride; Magnesium |
| Drops | | ingredients broken | Chloride; Potassium |
| | | down by active vs | Chloride; Purified Water; |
| | | inactive) | Sodium Borate; Sodium |
| | | | Chloride; Sodium |
| | | | Chlorite (OcuPure |
| | | | Brand) as a Preservative; |
| | | | Sodium Hyaluronate. |
| Blink GelTears | 10 mL | Polyethylene Glycol | Boric Acid; Calcium |
| Lubricating Eye | bottle | 400 0.25% | Chloride; Magnesium |
| Drops | | | Chloride;Potassium |
| | | | Chloride; Purified Water; |
| | | | Sodium Borate; Sodium |
| | | | Chloride; Sodium |
| | | | Chlorite (OcuPure brand) |
| | | | as a preservative; Sodium |
| | | | Hyaluronate. |
| Blink Tears | 15 mL | Polyethylene Glycol | Boric Acid; Calcium |
| Lubricating | bottle | 400 0.25% | Chloride; Magnesium |
| Eye Drops | | | Chloride; Potassium |
| | | | Chloride; Purified Water; |
| | | | Sodium Borate; Sodium |
| | | | Chloride; Sodium |
| | | | Chlorite (OcuPure |
| | | | Brand) as a Preservative; |
| | | | Sodium Hyaluronate. |
| Blink Tears | 25 0.4 mL | Polyethylene Glycol | Boric Acid; Calcium |
| Preservative | PF Vials | 400 0.25% | Chloride; Magnesium |
| Free Lubricating | | | Chloride; Potassium |
| Eye Drops | | | Chloride; Purified Water; |
| | | | Sodium Borate; Sodium |
| | | | Chloride; Sodium |
| | | | Hyaluronate. |
| Blink-N-Clean | 15 mL bottle | polyhexamethylene | Purified Water, Sodium |
| Lens Drops | | biguanide 0.0001%, | Chloride |
| | | Tromethamine | |
| | | (buffer), | |
| | | Hydroxypropyl | |
| | | Methylcellulose | |
| | | (lubricant), Tyloxapol | |
| | | (surfactant), Edetate | |
| | | Disodium (chelating | |
| | | agent) | |
| Clear Eyes | Bottle | Polyvinyl alcohol 0.5% | Benzalkonium Chloride, |
| Natural Tears | | Povidone 0.6% | Dextose, Edetate |
| Lubricant | | | Disodium, Potassium |
| | | | Chloride, Purified Water, |
| | | | Sodium Bicarbonate, |
| | | | Sodium Chloride, |
| | | | Sodium Citrate, Sodium |
| | | | Phosphate (mono- and |
| | | | dibasic) |
| Clear Eyes Outdoor | Bottle | Polyvinyl alcohol 0.5% | Benzalkonium Chloride, |
| Dry Eye | | Povidone 0.6% | Dextose, Edetate |
| Protection | | | Disodium, Potassium |
| | | | Chloride, Purified Water, |
| | | | Sodium Bicarbonate, |
| | | | Sodium Chloride, |
| | | | Sodium Citrate, Sodium |
| | | | Phosphate (mono- and |
| | | | dibasic) |
| Clear Eyes Pure | “240 drops” | Glycerin 0.25% | Boric acid, purified |
| Relief for | PF multi- | | water, sodium borate, |
| Dry Eyes | dose bottle | | sodium chloride, sodium |
| *DISCONTINUED* | | | hyaluronate |
| Contact lens | 15 mL bottle | Carmellose sodium | Borate Buffer, Sodium |
| comfort drops | | | Chloride, Potassium |
| (Akorn) | | | Chloride, Sodium |
| | | | Bicarbonate (baking |
| | | | soda), Calcium Chloride, |
| | | | Magnesium Chloride, |
| | | | Sodium Phosphate, |
| | | | Sodium Perborate |
| | | | stabilized with |
| | | | phosphonic acid |
| Freshkote | 10 mL PF | Polyvinyl pyrrolidone | Boric acid, Edetate |
| Preservative | bottle | 2.0%, Polyvinyl | disodium, Ethanol, |
| Free Multi- | | alcohol 2.7% | Glycerin, Lecithin, |
| dose (Focus) | | | Phospholipids, |
| | | | Polysorbate-80, |
| | | | Potassium chloride, |
| | | | Purified water, Sodium |
| | | | chloride. May contain |
| | | | hydrochloric acid and/or |
| | | | sodium hydroxide to |
| | | | adjust pH. |
| Freshkote | 30 0.3 mL | Polyvinyl pyrrolidone | Boric acid, Edetate |
| Preservative | single use | 2.0%, Polyvinyl | disodium, Ethanol, |
| Free Single | vials | alcohol 2.7% | Glycerin, Lecithin, |
| Use Vials | | | Phospholipids, |
| (Focus) | | | Polysorbate-80, |
| | | | Potassium chloride, |
| | | | Purified water, Sodium |
| | | | chloride. May contain |
| | | | hydrochloric acid and/or |
| | | | sodium hydroxide to |
| | | | adjust pH. |
| Genteal Gel | 10 g tube | Hypromellose 0.3%. | carbopol 980, phosphoric |
| (Severe)(Alcon) | | | acid, purifed water, |
| | | | sodium hydroxide, |
| | | | sodium perborate, and |
| | | | sorbitol |
| Genteal Mild | 15 mL/ | Hypromellose 0.2% | Boric acid, calcium |
| (Alcon) | 25 ml bottle | | chloride dihydrate, |
| *DISCONTINUED* | | | GenAqua (sodium |
| | | | perborate), phosphonic |
| | | | acid, potassium chloride, |
| | | | purified water, and |
| | | | sodium chloride |
| Genteal Moderate | 15 mL bottle | Dextran 70 0.1%, | Boric acid, calcium |
| (Alcon) | | Glycerin 0.2%, | chloride, glycine, |
| | | Hypromellose 0.3% | hydrochloric acid and/or |
| | | | sodium hydroxide (to |
| | | | adjust pH), magnesium |
| | | | chloride, POLYQUAD ® |
| | | | (polyquaternium-1) |
| | | | 0.001% preservative, |
| | | | polysorbate 80, |
| | | | potassium chloride, |
| | | | purified water, sodium |
| | | | chloride, zinc chloride. |
| Genteal | 3.5 g | White petrolatum 93% | anhydrous liquid lanolin |
| ointment(Alcon) | | Mineral oil 4% | |
| Genteal | 36 PF Vials | Dextran 70 0.1%, | Potassium Chloride, |
| Tears(Alcon)Previously | (0.9 mL) | Hypromellose 2910 | Purified Water, Sodium |
| Tears Naturale Free | | 0.3% | Borate, Sodium Chloride, |
| | | | May contain: |
| | | | Hydrochloric Acid, |
| | | | Sodium Hydroxide |
| Lubrifresh PM | 3.5 g tube | 83% petrolatum | Lanolin alcohol |
| | | 15% mineral oil | |
| Oasis Tears | 10 mL bottle | Glycerin (0.22%) | EDTA, potassium |
| (Oasis Medical) | | | sorbate, sodium chloride, |
| | | | sodium hyaluronate, |
| | | | sodium hydroxide, |
| | | | sodium phosphate |
| | | | dibasic, sodium |
| | | | phosphate monobasic, |
| | | | water |
| Oasis Tears(Oasis | 30 PF vials | Glycerin (0.2%) | Hyaluronan, sodium |
| Medical) | (0.3 mL) | | chloride, sodium |
| | | | phosphate dibasic, |
| | | | sodium phosphate |
| | | | monobasic, water. |
| Oasis Tears | 10 mL PF | Glycerin (0.25%) | sodium chloride, sodium |
| PF(Oasis | bottle | | hyaluronate, sodium |
| Medical) | | | hydroxide, sodium |
| | | | phosphate dibasic, |
| | | | sodium phosphate |
| | | | monobasic, water |
| Oasis Tears | 30 PF vials | Glycerin (0.2%) | Hyaluronan, sodium |
| Plus(Oasis | (0.3 mL) | | chloride, sodium |
| Medical) | | | phosphate dibasic, |
| | | | sodium phosphate |
| | | | monobasic, water. |
| Oasis Tears Plus | 10 mL PF | Glycerin (0.2%) | Sodium chloride, sodium |
| PF(Oasis Medical) | bottle | | hyaluronate, sodium |
| | | | hydroxide, sodium |
| | | | phosphate dibasic, |
| | | | sodium phosphate |
| | | | monobasic, water. |
| Optase Dry Eye | 10 mL (0.33 | Glycerin (0.2%) | Hydrochloric acid, |
| Intense Drops | oz) bottle | | sodium hyaluronate, |
| | | | sorbital, trometamol, |
| | | | water |
| Optase Dry | 17 mL (0.58 | Glycerin 1% | Sea buckthorn seed oil, |
| Eye Spray | oz) bottle | | sodium hyaluronate, |
| | | | trometamol-citrate |
| Optase Hylo | 5 g tube | White Petrolatum | Retinol palmitate |
| Night | | (90%), lanolin (6.9%) | (Vitamin A), light liquid |
| | | | paraffin |
| | | | May contain peanut oil |
| Optase MGD | 10 mL (0.33 | Glycerin 0.9% | Citric acid monohydrate, |
| Advanced | oz) bottle | | Polyoxyethylenesorbitan, |
| | | | Monooleate, Potassium |
| | | | Chloride, Purified Water, |
| | | | Sacha Inchi Seed Oil, |
| | | | Sodium hyaluronate, |
| | | | Sodium Monooleate, |
| | | | Trehalose, Tromethamine |
| Refresh | 30 PF vials | Carboxymethylcellulose | Calcium chloride, |
| Celluvisc(Allergan) | (0.4 mL) | sodium 1% | potassium chloride, |
| | | | purified water, sodium |
| | | | chloride, and sodium |
| | | | lactate. |
| Refresh Classic | 30 PF vials | Polyvinyl Alcohol | Purified water, sodium |
| (Allergan) | (0.4 mL) | 1.4% Povidone 0.6% | chloride. May also |
| | | | contain hydrochloric acid |
| | | | and/or sodium hydroxide |
| | | | to adjust pH. |
| Refresh Contacts | 12 mL bottle | Carboxymethylcellulose | sodium chloride, boric |
| (Allergan) | | sodium | acid, sodium borate |
| | | | decahydrate, potassium |
| | | | chloride, calcium |
| | | | chloride, magnesium |
| | | | chloride, purified water, |
| | | | and is preserved with |
| | | | PURITE ® (stabilized |
| | | | oxychloro complex). |
| | | | This formula may also |
| | | | contain hydrochloric acid |
| | | | and/or sodium hydroxide |
| | | | to adjust pH. |
| Refresh | 10 mL bottle | Carboxymethylcellulose | Boric acid; carbomer |
| Digital | | sodium 0.5% | copolymer type A; castor |
| | | Glycerin 1% | oil; erythritol; |
| | | Polysorbate 80 0.5% | levocarnitine; purified |
| | | | water; PURITE ® |
| | | | (stabilized oxychloro |
| | | | complex); and sodium |
| | | | hydroxide. |
| Refresh | 30 0.4 mL | Carboxymethylcellulose | Boricacid; carbomer |
| Digital PF | PF vials | sodium 0.5% | copolymer type A; castor |
| | | Glycerin 1% | oil; erythritol; |
| | | Polysorbate 80 0.5% | levocarnitine; purified |
| | | | water; and sodium |
| | | | hydroxide. |
| Refresh | 3.5 g tube | Mineral Oil | Chlorobutanol and |
| LacriLube(Allergan)OUT | | 42.5% White | lanolin alcohols. |
| OF PRODUCTION | | Petrolatum 56.8% | |
| Refresh Liquigel | 15 mL bottle | Carboxymethylcellulose | Boric acid, calcium |
| (Allergan) | | sodium 1% | chloride, magnesium |
| | | | chloride, potassium |
| | | | chloride, purified water, |
| | | | PURITE ® (stabilized |
| | | | oxychloro complex), |
| | | | sodium borate, and |
| | | | sodium chloride. |
| Refresh | 30 PF vials | Carboxymethylcellulose | Boric acid; calcium |
| Optive(Allergan) | (0.4 mL) | sodium 0.5%, | chloride dihydrate; |
| | | Glycerin 0.9% | erythritol; levocarnitine; |
| | | | magnesium chloride |
| | | | hexahydrate; potassium |
| | | | chloride; purified water; |
| | | | sodium borate |
| | | | decahydrate; and sodium |
| | | | citrate dihydrate. |
| | 15 mL bottle | | Boric acid; calcium |
| | | | chloride dihydrate; |
| | | | erythritol; levocarnitine; |
| | | | magnesium chloride |
| | | | hexahydrate; potassium |
| | | | chloride; purified water; |
| | | | PURITE ® (stabilized |
| | | | oxychloro complex); |
| | | | sodium borate |
| | | | decahydrate; and sodium |
| | | | citrate dihydrate. |
| Refresh Optive | 30 PF vials | Carboxymethylcellulose | Boric acid; carbomer |
| Advanced(Allergan) | (0.4 mL) | sodium 0.5% | copolymer type A; castor |
| | | Glycerin 1% | oil; erythritol; |
| | | Polysorbate 80 0.5% | levocarnitine; purified |
| | | | water; and sodium |
| | | | hydroxide. |
| | 10 mL bottle | | Boric acid; castor oil; |
| | | | erythritol; levocarnitine; |
| | | | carbomer copolymer type |
| | | | A; purified water; and |
| | | | PURITE ®(stabilized |
| | | | oxychloro complex). |
| | | | May also contain sodium |
| | | | hydroxide to adjust pH. |
| Refresh Optive | 10 mL bottle | Carboxymethylcellulose | Boric acid; calcium |
| Gel Drops | | sodium 1%, glycerin | chloride dihydrate; |
| (Allergan) | | 0.9% | erythritol; levocarnitine; |
| | | | magnesium chloride |
| | | | hexahydrate; potassium |
| | | | chloride; PURITE ® |
| | | | (stabilized oxychloro |
| | | | complex); sodium borate |
| | | | decahydrate; sodium |
| | | | citrate dihydrate; purified |
| | | | water. |
| Refresh Optive | 30 PF vials | Carboxymethylcellulose | Boric acid; butylated |
| MEGA-3(Allergan) | (0.4 mL) | sodium 0.5% | hydroxyl toluene; castor |
| | | Glycerin 1% | oil; erythritol; flaxseed |
| | | Polysorbate 80 0.5% | oil; levocarnitine; |
| | | | pemulen TR-2; polyoxyl |
| | | | 40 stearate; purified |
| | | | water; sodium hydroxide; |
| | | | trehalose. |
| Refresh | 30 PF vials | Carboxymethylcellulose | Calcium chloride; |
| Plus(Allergan) | (0.4 mL) | sodium 0.5% | magnesium chloride; |
| | | | potassium chloride; |
| | | | purified water; sodium |
| | | | chloride; and sodium |
| | | | lactate. May also contain |
| | | | hydrochloric acid and/or |
| | | | sodium hydroxide to |
| | | | adjust pH. |
| Refresh PM | 3.5 g tube | Mineral Oil | Lanolin alcohols |
| ointment(Allergan) | | 42.5% White | |
| | | Petrolatum 57.3% | |
| Refresh Relieva | 10 mL | carboxymethylcellulose | Boric acid; calcium |
| | (0.33 fl oz) | sodium 0.5% and | chloride dihydrate; |
| | multi-dose | glycerin 0.9% | erythitrol; levocarnitine; |
| | bottleAlso | | magnesium chloride |
| | sold in | | hexahydrate; potassium |
| | boxes of 30 | | chloride; purified water; |
| | PF vials | | sodium borate |
| | | | decahydrate; sodium |
| | | | citrate dihydrate; and |
| | | | sodium hyaluronate. May |
| | | | also contain hydrochloric |
| | | | acid and/or sodium |
| | | | hydroxide to adjust pH. |
| Refresh Tears | 15 mL bottle | Carboxymethylcellulose | Oxychloro complex, |
| (Allergan) | | Sodium | Sodium Borate |
| | | | decahydrate, Sodium |
| | | | chloride, Potassium |
| | | | chloride, Calcium |
| | | | chloride dihydrate, |
| | | | Magnesium chloride |
| | | | hexahydrate, Boric acid, |
| | | | Sodium hydroxide to |
| | | | adjust pH and Purified |
| | | | water. |
| Retaine CMC | 30 PF vials | Carboxymethylcellulos | Calcium chloride, |
| (Ocusoft) | (0.6 mL) | e sodium 0.5% | magnesium chloride, |
| | | | potassium chloride, |
| | | | purified water, sodium |
| | | | chloride, sodium lactate. |
| | | | May also contain |
| | | | hydrochloric acid and/or |
| | | | sodium hydroxide to |
| | | | adjust pH. |
| Retaine | 10 mL bottle | Hypromellose 0.3% | Citric acid, sodium |
| HPMC(Ocusoft) | (preservative | | citrate, sorbitol, water |
| | free) | | for injection. |
| Retaine | 30 PF vials | Light Mineral Oil | Cetalkonium chloride, |
| MGD(Ocusoft)(Equivalent | (0.4 mL) | (0.5%) Mineral Oil | glycerol, poloxamer 188, |
| to Cationorm which is | | (0.5%) | tris hydrochloride, |
| sold in Europe) | | | tromethamine, tyloxapol, |
| | | | water for injection. |
| Retaine PM | 5 g tube | Mineral oil (20%) | none (one of the few |
| ointment(Ocusoft) | | White petrolatum | ointments that does not |
| | | (80%) | contain lanolin alcohol) |
| Soothe Long Lasting | 15 mL bottle | Povidone (2.0%) | benzalkonium chloride |
| Hydration | | | (0.005%), boric acid, |
| (Bausch & Lomb) | | | edetate |
| | | | disodiumdihydrate, |
| | | | purified water, sodium |
| | | | borate, sodium chloride, |
| | | | tyloxapol. Hydrochloric |
| | | | acid and/or sodium |
| | | | hydroxide may be used |
| | | | to adjust pH. |
| Soothe Lubricant Eye | 15 mL bottle | Povidone (1.25%) | boric acid, potassium |
| Drops Hydration | | | chloride, sodium borate |
| (Bausch & Lomb) | | | and sodium |
| | | | chloride;preserved with |
| | | | edetate disodium 0.1% |
| | | | and sorbic acid 0.1% |
| Soothe Lubricant | 28 PF Vials | Glycerin (0.6%) | boric acid, |
| Eye Drops | (0.6 mL) | Propylene Glycol | hydroxyalkylphosphonate, |
| Preservative Free | | (0.6%) | purified water, sodium |
| (Bausch & Lomb) | | | alginate, sodium borate |
| Soothe Maximum | 15 mL bottle | Povidone (2.0%) | benzalkonium chloride |
| Hydration | | | (0.005%), boric acid, |
| (Bausch & Lomb) | | | edetate |
| | | | disodiumdihydrate, |
| | | | purified water, sodium |
| | | | borate, sodium chloride, |
| | | | tyloxapol. Hydrochloric |
| | | | acid and/or sodium |
| | | | hydroxide may be used |
| | | | to adjust pH. |
| Soothe Night | 3.5 g tube | Mineral oil (20%), | None. |
| Time Ointment | | White petrolatum | Note - this is one of the |
| (Bausch & Lomb) | | (80%) | only lubricant ointments |
| | | | on the list WITHOUT |
| | | | lanolin alcohol |
| | | | (Jun. 14, 2018) |
| Soothe XP Emollient | 15 mL bottle | Light mineral oil | boric acid, edetate |
| (Bausch & Lomb) | | (1.0%), Mineral oil | disodium, octoxynol-40, |
| | | (4.5%) | polyquaternium-1 |
| | | | (preservative), |
| | | | polysorbate 80, purified |
| | | | water, sodium borate |
| | | | decahydrate. May |
| | | | contain hydrochloric acid |
| | | | and/or sodium hydroxide |
| | | | (to adjust pH). |
| Soothe XP | 30 0.3 mL | Light mineral oil | boric acid, edetate |
| Emollient PF | preservative- | (1.0%), Mineral oil | disodium, octoxynol-40, |
| (Bausch & Lomb) | free vials | (4.5%) | polysorbate 80, purified |
| | | | water, sodium borate |
| | | | decahydrate. May |
| | | | contain hydrochloric acid |
| | | | and/or sodium hydroxide |
| | | | (to adjust pH). |
| Systane | 28 PF vials | Polyethylene Glycol | Boric acid, calcium |
| (Alcon) | (0.4 mL) | 400 0.4%, Propylene | chloride, hydroxypropyl |
| | | Glycol 0.3% | guar, magnesium |
| | | | chloride, potassium |
| | | | chloride, purified water, |
| | | | sodium chloride, zinc |
| | | | chloride. May contain |
| | | | hydrochloric acid and/or |
| | | | sodium hydroxide to |
| | | | adjust pH. |
| | 15 mL bottle | | Boric acid, calcium |
| | | | chloride, hydroxypropyl |
| | | | guar, magnesium |
| | | | chloride, |
| | | | polyquaternium-1 as a |
| | | | preservative, potassium |
| | | | chloride, purified water, |
| | | | sodium chloride, zinc |
| | | | chloride. May contain |
| | | | hydrochloric acid and/or |
| | | | sodium hydroxide to |
| | | | adjust pH. |
| Systane Balance | 10 mL bottle | Propylene Glycol 0.6% | Boric acid, dimyristoyl |
| (Alcon) | | | phosphatidylglycerol, |
| | | | edetate disodium, |
| | | | hydroxypropyl guar, |
| | | | mineral oil, polyoxy1 40 |
| | | | stearate, POLYQUAD ® |
| | | | (polyquaternium-1) |
| | | | 0.001% preservative, |
| | | | sorbitan tristearate, |
| | | | sorbitol and purified |
| | | | water. May contain |
| | | | hydrochloric acid and/or |
| | | | sodium hydroxide to |
| | | | adjust pH. |
| Systane Complete | 10 mL bottle | Propylene Glycol 0.6% | Boric acid, dimyristoyl |
| (Alcon) | (1/3 fl oz) | | phosphatidylglycerol, |
| | | | edetate disodium, |
| | | | hydroxypropyl guar, |
| | | | mineral oil, polyoxl 40 |
| | | | Stearate, POLYQUAD |
| | | | (polyquaternium-1) |
| | | | 0.001% preservative, |
| | | | sorbitan tristearate, |
| | | | sorbital and purified |
| | | | water. May contain |
| | | | hydrochloric acid and/or |
| | | | sodium hydroxide to |
| | | | adjust pH. |
| Systane Complete | 10 mL PF | Propylene Glycol 0.6% | Boric acid, dimyristoyl |
| Preservative Free | bottle | | phosphatidylglycerol, |
| Multidose (Alcon) | (1/3 fl oz) | | hydroxypropyl guar, |
| | | | mineral oil, polyoxl 40 |
| | | | stearate, sorbitan |
| | | | tristearate, sorbital and |
| | | | purified water. May |
| | | | contain hydrochloric acid |
| | | | and/or sodium hydroxide |
| | | | to adjust pH. |
| Systane Contacts | 12 mL bottle | dextran, hydroxypropyl | sodium chloride, |
| (Alcon) | | methylcellulose | potassium chloride, with |
| | | | edetate disodium 0.1% |
| | | | and POLYQUAD ® |
| | | | (polyquaternium-1) |
| Systane Day & Night | Two 10 mL | See Systane Gel Drops | See Systane Gel Drops |
| (Alcon) | bottles | and Systane Ultra | and Systane Ultra |
| | (Systane | | |
| | Gel Drops | | |
| | and Systane | | |
| | Ultra) | | |
| Systane Gel Drops | 10 mL bottle | Polyethylene Glycol | Aminomethylpropanol, |
| (Alcon) | | 400 0.4%, Propylene | boric acid, edetate |
| | | Glycol 0.3% | disodium, hydroxypropyl |
| | | | guar, POLYQUAD ® |
| | | | (polyquaternium-1) |
| | | | 0.001% preservative, |
| | | | potassium chloride, |
| | | | sodium chloride, sorbitol |
| | | | and purified water. May |
| | | | contain hydrochloric acid |
| | | | and/or sodium hydroxide |
| | | | to adjust pH. |
| Systane | Box of 30 | Polyethylene Glycol | aminomethylpropanol, |
| Hydration PF | 10 mL vials | 400 0.4%, Propylene | boric acid, |
| | | Glycol 0.3% | hydroxypropyl guar, |
| | | | potassium chloride, |
| | | | purified water, sodium |
| | | | borate, sodium chloride, |
| | | | sodium hyaluronate, |
| | | | sorbitol. May contain |
| | | | hydrochloric acid and/or |
| | | | sodium hydroxide to |
| | | | adjust pH. |
| Systane Hydration | 10 mL bottle | Polyethylene Glycol | aminomethylpropanol, |
| Multidose | | 400 0.4%, Propylene | boric acid, |
| Preservative-Free | | Glycol 0.3% | hydroxypropyl guar, |
| | | | potassium chloride, |
| | | | purified water, sodium |
| | | | borate, sodium chloride, |
| | | | sodium hyaluronate, |
| | | | sorbitol. May contain |
| | | | hydrochloric acid and/or |
| | | | sodium hydroxide to |
| | | | adjust pH. |
| Systane Lubricant | 10 g | Hypromellose (0.3%) | Carbopol 980, |
| Eye Gel(Alcon) | | | phosphonic acid, purified |
| | | | water, sodium perborate |
| | | | and sorbitol. May contain |
| | | | sodium hydroxide to |
| | | | adjust pH. |
| Systane Nighttime | 3.5 g tube | White petrolatum 94% | Anhydrous Liquid |
| ointment(Alcon) | | Mineral oil 3% | Lanolin 3%. |
| Systane Sport | Two 10 mL | Polyethylene Glycol | Aminomethylpropanol, |
| (Alcon) | bottles | 400 0.4% Lubricant | boric acid, |
| | | Propylene Glycol 0.3% | hydroxypropyl guar, |
| | | | POLYQUAD ® |
| | | | (polyquaternium-1) |
| | | | 0.001% preservative, |
| | | | potassium chloride, |
| | | | purified water, sodium |
| | | | chloride, sorbitol. May |
| | | | contain hydrochloric acid |
| | | | and/or sodium hydroxide |
| | | | to adjust pH. |
| Systane | 25 PF vials | Polyethylene Glycol | Aminomethylpropanol, |
| Ultra(Alcon) | (0.7 mL) | 400 0.4%, Propylene | boric acid, |
| | 60 PF vials | | hydroxypropyl guar, |
| | (0.7 mL) | | potassium chloride, |
| | | | purified water, sodium |
| | | | chloride, sorbitol. May |
| | | | contain hydrochloric acid |
| | | | and/or sodium hydroxide |
| | | | to adjust pH. |
| | 10 mL bottle | Glycol 0.3% | Aminomethylpropanol, |
| | | | boric acid, |
| | | | hydroxypropyl guar, |
| | | | POLYQUAD* |
| | | | (polyquaternium-1) |
| | | | 0.001% preservative, |
| | | | potassium chloride, |
| | | | purified water, sodium |
| | | | chloride, sorbitol. May |
| | | | contain hydrochloric acid |
| | | | and/or sodium hydroxide |
| | | | to adjust pH. |
| Thera Tears | 30 mL bottle | Sodium | Borate Buffers, Calcium |
| dry eye | | Carboxymethyl | Chloride, Dequest, |
| therapy (Akorn) | | | Magnesium Chloride, |
| | | | Potassium Chloride, |
| | | | Purified Water, Sodium |
| | | | Bicarbonate, Sodium |
| | | | Chloride, Sodium |
| | | | Perborate, Sodium |
| | | | Phosphate |
| | 32 PF vials | Cellulose (0.25%) | Borate Buffers, Calcium |
| | (0.6 mL) | | Chloride, Magnesium |
| | | | Chloride, Potassium |
| | | | Chloride, Purified Water, |
| | | | Sodium Bicarbonate, |
| | | | Sodium Chloride and |
| | | | Sodium Phosphate. |
| Thera Tears | 15 mL bottle | Sodium | Boric Acid Calcium |
| EXTRA Dry | | Carboxymethyl | Chloride, Dequest, |
| Eye Therapy | | Cellulose (0.25%) | Potassium Chloride, |
| | | | Sodium Bicarbonate, |
| | | | Sodium Borate, Sodium |
| | | | Chloride, Magnesium |
| | | | Chloride, Sodium |
| | | | Perborate, Sodium |
| | | | Phosphate Trehalose, |
| | | | Water for Injection |
| TheraTears | 28 PF vials | Sodium | Borate Buffer, Calcium |
| nighttime dry | (0.6 mL) | Carboxymethyl | Chloride, Magnesium |
| eye therapy | | Cellulose (1%) | Chloride, Potassium |
| (gel) (Akorn) | | | Chloride, Water |
| | | | (Purified), Sodium |
| | | | Bicarbonate (Baking |
| | | | Soda), Sodium Chloride, |
| | | | Sodium Phosphate |
| Visine Dry | 15 mL bottle | Glycerin 0.2% | ascorbic acid, |
| Eye Relief | | Hypromellose 0.2% | benzalkomium chloride, |
| | | Polyethylene glycol | boric acid, dextrose, |
| | | 400 1% | disodium phosphate, |
| | | | glycine, magnesium |
| | | | chloride, potassium |
| | | | chloride, purified water, |
| | | | sodium borate, sodium |
| | | | chloride, sodium citrate, |
| | | | sodium lactate |
| Visine Tired | 15 mL bottle | Glycerin 0.2% | ascorbic acid, boric |
| Eye Dry Eye | | Hypromellose 0.36% | acid, dextrose, glycine, |
| Relief | | Polyethylene glycol | magnesium chloride, |
| | | 400 1% | polyquaternium-42, |
| | | | potassium chloride, |
| | | | purified water, sodium |
| | | | borate, sodium chloride, |
| | | | sodium citrate, sodium |
| | | | lactate, sodium |
| | | | phosphate dibasic |
| iVIZIA Sterile | 0.33 fl oz | Povidone 0.5% | |
| Lubricant | bottle | | |
| Eye Drops for | | | |
| Dry Eyes, | | | |
| Preservative-Free | | | |

Non-limiting examples of ophthalmic eye drops that can be formulated with an ai-amylase of the disclosure include:

TABLE 13

| | | | Concentration |
|---|---|---|---|
| Supplier | Name | Preservative | (where stated) |
| Bausch + | Advanced Eye Relief | Benzalkonium | 0.010% |
| Lomb | Dry Eye Rejuvenation | chloride | |
| | Lubricant eye drops | | |
| | Soothe XP Emolient Eye | Polyquad | |
| | Drops | | |
| Soothe | Lubricant Eye Drops - | Benzalkonium | 0.005% |
| | Maximum Hydration | chloride | |
| | Hydration LubricantEye | Edetate | 0.1% |
| | Drops | disodium | |
| | | Sorbic acid | 0.1% |
| Clear Eyes | Natural Tears Lubricant | Benzalkonium | |
| | | chloride | |
| Johnson & | Visine Dry Eye Tired | Benzalkonium | |
| Johnson | Eye Relief | chloride | |
| Vision | | | |
| Prestige | Murine Tears for Dry | Benzalkonium | |
| Consumer | Eyes | chloride | |
| Healthcare | | | |
| Alcon | ISOPTO* TEARS 0.5% | Benzalkonium | 0.010% |
| | | chloride | |
| | Systane Original | Polyquad | 0.001% |
| | Systane Ultra | Polyquad | 0.001% |
| | Systane Ultra Hydration | Polyquad | 0.001% |
| | Systane Balance | Polyquad | 0.001% |
| | Systane Complete | Polyquad | 0.001% |
| | Systane Gel Drops | Polyquad | 0.001% |
| | Systane Lubricant Eye | Sodium | |
| | Gel | perborate | |
| | GenTeal | GenAqua | |
| | | (sodium | |
| | | perborate) | |
| | Tears Naturale II | Polyquad | 0.001% |
| | Tears Naturale Forte | Polyquad | 0.001% |
| Allergan | Refresh Repair | Purite | |
| | | (stabilized | |
| | | oxychloro | |
| | | complex) | |
| | Refresh Optive | Purite | |
| | | (stabilized | |
| | | oxychloro | |
| | | complex) | |
| | Refresh Optive Gel | Purite | |
| | Drops | (stabilized | |
| | | oxychloro | |
| | | complex) | |
| | Refresh Optive | Purite | |
| | Advanced | (stabilized | |
| | | oxychloro | |
| | | complex) | |
| | Refresh Tears | Purite | |
| | | (stabilized | |
| | | oxychloro | |
| | | complex) | |
| Refresh | Purite (stabilized | | |
| Liquigel | oxychloro complex) | | |
| Focus | FreshKote Lubricant Eye | Polixetonium | |
| Laboratories | Drops | (Polyquaternium-42) | |
| Johnson and | Blink Tears Lubricating | OcuPure (stabilized | |
| Johnson | Eye Drops | oxychloro complex) | |
| Vision | Blink Gel Tears | OcuPure (stabilized | |
| | Lubricating Eye Drops | oxychloro complex) | |
| Visine Dry | Polyquaternium-42 | | |
| Eye Tired | | | |
| Eye Relief | | | |
| Thera Tears | Extra Dry Eye Therapy | Dequest (sodium perborate) | |
| Dry Eye | Dequest (sodium | | |
| Therapy | perborate) | | |

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Example 1. Opthalmic Demulcent Composition

This example describes the procedure for testing a composition comprising a therapeutically effective concentration of an α-amylase enzyme from Aspergillus oryzae with various ophthalmic demulcents. The rationale for selection of the following ophthalmic demulcents is as follows:

### Selection of Demulcents

### Cellulose Derivatives (Demulcent Compositions 1-4):

Aspergillus oryzae α-amylase (E.C. 3.2.1.1) catalyzes endohydrolysis of 1,4-alpha-D-glucosidic linkages in polysaccharides. While cellulose is made of polysaccharides, because they are linked by β (1→4) glucan linkages, α-amylase is not believed to break such bonds, and may become inhibited by bonding to it. This study will evaluate if cellulose derivatives are suitable for use as demulcent's for compositions containing α-amylase, or if α-amylase activity is inhibited.

### Dextran:

Dextran is a branched glucan polysaccharide comprising α-1,6 glycosidic linkages. Notably, dextran is different from a dextrin which is a polysaccharide that can comprise either α-1,4 or α-1,6 linkages. This study will evaluate if dextran is suitable for use as a demulcent in compositions containing α-amylase.

### Gelatin:

Gelatin is comprised of proteins and peptides and is not expected to interact functionally with α-amylase. This study will evaluate if gelatin is suitable for use as a demulcent in compositions containing α-amylase.

### Polyols:

It is hypothesized that addition of polyols to an aqueous of α-amylase may increase the thermal stability of α-amylase in solution. This study will evaluate if polyols are suitable for use as a demulcent in compositions containing α-amylase.

### Polyvinyl Alcohol:

Polyvinyl alcohol is a biocompatible, water soluble, synthetic polymer and should not interact with α-amylase. This study will evaluate if polyvinyl alcohol is suitable for use as a demulcent in compositions containing α-amylase.

### Povidone:

Povidone is a biocompatible, water soluble, polymer and should not interact with α-amylase. This study will evaluate if povidone is suitable for use as a demulcent in compositions containing α-amylase.

Compositions tested in this study are as follows:

TABLE 14

| α-amylase + Demulcent | |
|---|---|
| Composition | Key Components (enzyme + ophthalmic demulcent) |
| Composition 1 | α-amylase + Carboxymethylcellulose sodium, 0.2% to 2.5% |
| | (percent volume) |
| Composition 2 | α-amylase + Hydroxyethyl cellulose, 0.2% to 2.5% (percent |
| | volume) |
| Composition 3 | α-amylase + Hypromellose, 0.2% to 2.5% (percent volume) |
| Composition 4 | α-amylase + Methylcellulose, 0.2% to 2.5% (percent volume) |
| Composition 5 | α-amylase + Dextran 70, 0.1% (percent volume) |
| Composition 6 | α-amylase + Gelatin, 0.01% (percent volume) |
| Composition 7 | α-amylase + Glycerin, 0.2% to 1% (percent volume) |
| Composition 8 | α-amylase + Polyethylene glycol 300, 0.2% to 1% (percent |
| | volume) |
| Composition 9 | α-amylase + Polyethylene glycol 400, 0.2% to 1% (percent |
| | volume) |
| Composition 10 | α-amylase + Polysorbate 80, 0.2% to 1% (percent volume) |
| Composition 11 | α-amylase + Propylene glycol, 0.2% to 1% (percent volume) |
| Composition 12 | α-amylase + Polyvinyl alcohol, 0.1% to 4% (percent volume) |
| Composition 13 | α-amylase + Povidone, 0.1% to 2% (percent volume) |

### 1.1 Composition Development—Demulcent Characterization

Compositions of α-amylase in combination with the aforementioned ophthalmic demulcents are made by using preservative free commercial eye drops in a buffered saline solution. Notably, α-amylase is believed to require calcium ions to function and compositions of the disclosure include calcium ions. Note: CaCl₂ concentration may be adjusted in protocol based on Ca Cofactor test results. If excessive Ca results in inactivation of enzymes the concentration can be adjusted.

Compositions comprising different preservatives are tested in a different experiment, described in Example 2.

1.1.1 Composition Development—α-Amylase Formulation with Co-Factors (No Demulcent):
Step 1: Prepare a 10 ml solution of 10 mg/ml α-amylase with cofactors. Add 100 mg of α-amylase from Aspergillus oryzae (Sigma-Aldrich Catalog #10065) to 10 ml sterile DI water in a flask. Mix until α-amylase is dissolved and evenly distributed.
Step 2: Dissolve 40 mg of calcium chloride and 56 mg of sodium chloride into the solution. Mix until all components are dissolved and evenly distributed.

### 1.1.2 Composition Development—α-Amylase Formulation+Cellulose Derivative Demulcents (Compositions 1-4):

Protocol for preparing a 10 ml solution of 10 mg/ml α-amylase in saline with Carboxymethylcellulose sodium 0.5%. Note: Carboxymethylcellulose sodium can be substituted with hydroxyethyl cellulose, Hypromellose, or methylcellulose in the ranges described in the above Table.
Step 1: Add 100 mg of α-amylase from Aspergillus oryzae (Sigma-Aldrich Catalog #10065) to flask. Add 40 mg of calcium chloride and 56 mg of sodium chloride to flask. Mix until all components are dissolved and evenly distributed.
Step 2: Add 50 mg of Carboxymethylcellulose sodium (Sigma-Aldrich Catalog #419273) (or another appropriate cellulose derivative described herein) and mix until dissolved.

### 1.1.3 Composition Development—α-Amylase Formulation+Dextran Demulcents (Composition 5):

Protocol for preparing a 10 ml solution of 10 mg/ml α-amylase in saline with Dextran 70 0.1%, Hypromellose 0.3%
Step 1: add 20 mg of α-amylase from Aspergillus oryzae (Sigma-Aldrich Catalog #10065) to 2 ml ALCON GenTeal Tears Preservative Free Single Use Vials in a flask. Mix until all components are dissolved and evenly distributed.
Step 2: dissolve 0.006 g of calcium chloride into the solution., mix until dissolved.

### 1.1.4 Composition Development—α-Amylase Formulation+Gelatin (Composition 6):

Protocol for preparing a 2 ml solution of 10 mg/ml α-amylase in saline with Gelatin 0.01%:
Step 1: Add 0.2 mg pure gelatin powder to 2 ml room temperature saline in a flask, do not mix.
Step 2: Bloom gelatin for 5 min.
Step 3: Warm flask to 50 C and mix gelatin until dissolved
Step 4: Cool flask to room temperature before proceeding.
Step 5: Add 20 mg of α-amylase from Aspergillus oryzae (Sigma-Aldrich Catalog #10065) to flask. Mix until α-amylase is dissolved and evenly distributed.

Dissolve 0.006 g of calcium chloride into the solution.

### 1.1.5 Composition Development—α-Amylase Formulation+Glycerin (Composition 7):

Protocol for preparing a 10 ml solution of 10 mg/ml α-amylase in saline with Glycerin 0.5%
Step 1: Add 100 mg of α-amylase from Aspergillus oryzae (Sigma-Aldrich Catalog #10065) to flask. Add 40 mg of calcium chloride and 56 mg of sodium chloride to flask. Mix until α-amylase is dissolved and evenly distributed.
Step 2: Add 100 mg of glycerin to flask. Fill flask to 10 ml mark with sterile DI water. Mix until all components are dissolved and evenly distributed.

### 1.1.6 Composition Development—α-Amylase Formulation in Saline+Polyols (Compositions 8-11):

### Polyethylene Glycol 400-1.0%

Step 1: Add 100 mg of α-amylase from Aspergillus oryzae (Sigma-Aldrich Catalog #10065) to flask. Add 40 mg of calcium chloride and 56 mg of sodium chloride to flask.

Mix until α-amylase is dissolved and evenly distributed.
Step 2: Add 100 mg of Polyethylene glycol 400 (Sigma-Aldrich Catalog #8074850050) and mix until dissolved. Fill flask to 10 ml mark with sterile DI water. Mix until all components are dissolved and evenly distributed.

Note: Polyethylene Glycol 400 can be substituted with Polyethylene glycol 300 and other suitable polyols.

### Propylene Glycol 1.0%

Step 1: Add 100 mg of α-amylase from Aspergillus oryzae (Sigma-Aldrich Catalog #10065) to flask. Add 40 mg of calcium chloride and 56 mg of sodium chloride to flask.
Step 2: Add 100 mg of propylene glycol to flask. Fill flask to 10 ml mark with sterile DI water. Mix until all components are dissolved and evenly distributed.

### Polyvinyl Alcohol 4% (PVA)

Step 1: 4% PVA solution (4 g PVA per 100 ml DI water) should be made ahead of time as PVA must be added to room temperature DI water in a gently capped media bottle with a magnetic stir, gently heated while being stirred continuously to 90 C, held at 90 C for one hour, and brought back to room temperature.
Step 2: Add 100 mg of α-amylase from Aspergillus oryzae (Sigma-Aldrich Catalog #10065) to 10 ml 4% PVA solution. Mix until α-amylase is dissolved and evenly distributed.
Step 3: Dissolve 40 mg of calcium chloride and 56 mg of sodium chloride into the solution. Mix until all components are evenly dissolved.

### 1.2 Composition Assessment—Well Plate Set Up for In-Vitro Testing of Inhibition of Biofilm Formation

Using the protocol listed below to prepare and plate Staph. Aureus for biofilm formation protocol.

### 1.2.1 Thaw S. Aureus

Thaw the bacterial strain upright using gentle agitation in a 25° C. to 30° C. water bath. Thawing will be rapid; approximately 2-3 minutes or until all ice crystals have melted.

Immediately after thawing, wipe down the mini-cryovial with 70% ethanol and aseptically inoculate a vented sterile culture tube for aerobic bacteria with 10 mL of Tryptic Soy broth (30 g per 1000 ml DI H₂O) using a sterile inoculating loop. Incubate at 37° C. for 24 hours in a shaking incubator.

### 1.2.2 Prepare Culture for Plating

Step 1. Place a new CuveTip on the Photopette
Step 2. Turn on the Photopette and ensure the bluetooth has connected to the iPad.
Step 3. Open the App and set the measurement to collect at 600 nm (use ecoli setting for brevity).
Step 4. Zero the Photopette in DI water, and clean the tip with a chemwipe.
Step 5. Aseptically transfer 100 ml of the TSB citrate media (TSBC) to a flask.
Step 6. Aseptically transfer the overnight culture of Staphylococcus aureus subsp. aureus Rosenbach (ATCC 29213) in Tryptic Soy Broth to the TSB citrate media (TSBC) using a pipet until the culture is diluted to A600 of 0.02 (~2×10\hat{ }8 CFU/ml) measured with the Photopette.
Step 7. Remove a sterile 96 well plate from the sterile packaging in the hood and place on the table.
Step 8. Set an 8 channel pipet for 100l and attach sterile tips.
Step 9. Plate the desired number of wells with 1001 of the diluted culture.
Step 10. Cover the well plate and incubate overnight for 18 hrs at 37° C.

### 1.2.3 Plating

Plate S. Aureus in all wells of a 96 tissue culture treated well plate. The well plate should incubate for 18 hrs at 37° C. before being assayed for biofilm formation.

### 1.3 Composition Testing

### 1.3.1 Test Compositions Day 1

Carefully tilt plate and pipet all liquid from the wells by inserting the pipet tips in the bottom corner of the wells to remove non-adherent cells without disturbing the biofilm grown on the bottom of the well.

Start a timer for 10 minutes when the first row is treated using an 8-channel pipette. Each row should be treated every 30 seconds making sure to gently release the composition in the corner of the well to not disturb the biofilm mechanically.

For Row 1: add 50 μl of 10 mg/ml α-amylase with cofactors to all wells.

For Row 2: add 50 μl of 10 mg/ml α-amylase formulated with Polyethylene Glycol 400 1.0% to all wells.

For Row 3: add 50 μl of 10 mg/ml α-amylase formulated with Carboxymethylcellulose sodium 2.5% to all wells.

For Row 4: add 50 μl of 10 mg/ml α-amylase formulated with Glycerin 1.0% to all wells.

For Row 5: add 50 μl of 10 mg/ml α-amylase formulated with Povidone 2.0% to all wells.

For Row 6: add 50 μl of 10 mg/ml α-amylase formulated with Propylene Glycol 1.0% to all wells.

For Row 7: add 50 μl of 10 mg/ml α-amylase formulated with Polyvinyl alcohol 2.7% to all wells.

For Row 8: add 50 μl of 10 mg/ml α-amylase in saline with Dextran 70 0.1%, Hypromellose 2910 0.3% to all wells.

For Row 9: add 50 DI water to all well. Do not treat Rows 11-12. Incubate plate for 10 minutes at 37° C.

### 1.4 Assessment of Biofilm Reduction

After 10 min all fluid should be removed row by row every 30 seconds starting with Row 1. The fluid must be removed with gentle pipetting, with the plate at an angle, with the pipette in the corner to remove non-adherent cells without disturbing the biofilm grown on the bottom of the well.

Add 50 μl of 0.1% crystal violet (CV) to stain adherent cells for all wells.

Carefully rinse all wells by submerging the plate in a DI water bath to remove all remaining crystal violet.

Dry the well plate and measure the optical density of the biofilms using a plate reader at 600 nm.

Once all measurements have been taken the well plate should be transferred to a bleach solution for sanitization and disposed of in a biological waste bin.

### 1.5 Assessment of Preservative Effects of Various Compositions

Repeat the steps outlined in 1.2 and 1.3 for scheduled testing days on days 7, 14, 21, 28, and 35. For each day of testing scheduled at days 7, 14, 21, 28, and 35 a well plate of biofilm should be prepared the day before.

Once all measurements have been taken the well plate should be transferred to a bleach solution for sanitization and disposed of in a biological waste bin. All experiments are performed using sterile techniques with sterile equipment in a clean environment.

### Example 2. Opthalmic Preservative Composition Testing

This example describes the procedure for testing compositions of α-amylase from Aspergillus oryzae with ophthalmic preservatives for compatibility. These preservatives tested are intended to prohibit microbial growth, not extend the shelf life on α-amylase.

2.1 Composition Development—Evaluation of Preservative on α-amylase function

The present disclosure postulates that S. Aureus biofilms on the eyelid are a major cause of dry eye disease. α-amylase has not been used previously to remove biofilms from the eyelid. The present disclosure describes the testing and characterization of various combinations of ophthalmic solution ingredients with α-amylase for efficacy in removing biofilms in cell culture before testing these solutions on tissue. This experiment aims to evaluate whether ophthalmic preservatives help or hinder the effectiveness of α-amylase in the reduction of S. Aureus biofilms. The preservatives below are evaluated in this example:

A) BAK generally functions as a detergent action for dissolving cell walls and membranes, however it is unclear if it affects α-amylase stability/function. B) Polyquatenium-1 is believed to act on cell membranes, similarly it is unclear if it affects α-amylase stability/function. C) Stabilized oxychloro complex(es) are believed to function as preservatives via oxidation of intracellular lipids and glutathione, similarly it is unclear if it affects α-amylase stability/function. D) Sodium perborate is believed to function by forming hydrogen peroxide, oxidizing action, however it is unknown if it affects α-amylase stability/function. E) Edetate disodium and Sorbic acid, or Edetate disodium, or Ethylenediaminetetraacetic acid (EDTA) are believed to function by binding to heavy metals such as Iron or Calcium. Since α-amylase uses Calcium as a cofactor, this protocol will test if these preservatives affect α-amylase stability/function. F) Polixetonium (Polyquaternium-42) is uncommonly used as a preservative, it is unclear if it affects α-amylase stability/function. G) Borate, sorbitol, propylene glycol and zinc Ionic buffer may function via multiple potential methods of action, this protocol will test if these preservatives affect α-amylase stability/function. H) Polyhexanide is believe to work by impairing bacterial cell membrane activity, this protocol will test if these preservatives affect a-amylase stability/function.

TABLE 15

| α-amylase + Preservative | |
|---|---|
| Composition | Key Components (enzyme + ophthalmic preservative) |
| Composition 14 | α-amylase + BAK (benzalkonium chloride) 0.01% |
| Composition 15 | α-amylase + Polyquad (polyquaternium-1) 0.001% |
| Composition 16 | α-amylase + Purite/OcuPure (stabilized oxychloro complex) |
| Composition 17 | α-amylase + GenAqua/Dequest (sodium perborate) |
| Composition 18 | α-amylase + Edetate disodium 0.1% and Sorbic acid 0.1% |
| Composition 19 | α-amylase + Polixetonium (Polyquaternium-42) |
| Composition 20 | α-amylase + SofZia (Borate, sorbitol, propylene glycol |
| | and zinc ionic buffer) |
| Composition 21 | α-amylase + Polyhexanide (polyhexamethylene biguanide) |

2.1.1 Composition Development—α-Amylase Formulation with Cofactors (No Preservative—Control Composition):

Step 1: add 100 mg of α-amylase from Aspergillus oryzae (Sigma-Aldrich Catalog #10065) to 10 ml sterile DI Water in a flask. Mix until α-amylase is dissolved and evenly distributed.

Step 2: Dissolve 40 mg of calcium chloride and 56 mg of sodium chloride into the solution. Mix until all ingredients are dissolved and evenly distributed.

### 2.1.2 Composition Development—α-Amylase Formulation+Benzalkonium Chloride (Composition 14):

Protocol for preparing a 10 ml solution of 10 mg/ml α-amylase with benzalkonium chloride 0.01%:

Step 1: Add 100 mg of α-amylase from Aspergillus oryzae (Sigma-Aldrich Catalog #10065) to 10 ml benzalkonium chloride (Sigma-Aldrich Catalog #PHR1371) diluted with DI water to 0.01% in a flask. Mix until α-amylase is dissolved and evenly distributed.

Step 2: Dissolve 40 mg of calcium chloride and 56 mg of sodium chloride into the solution.

### 2.1.3 Composition Development—α-Amylase Formulation+Polyquad (Polyguaternium-1) (Composition 15):

Protocol for preparing a 10 ml solution of 10 mg/ml α-amylase with Polyquad 0.001%:

Step 1: Add 100 mg of α-amylase from Aspergillus oryzae (Sigma-Aldrich Catalog #10065) to 2 ml of Systane original in a flask. Mix until α-amylase is dissolved and evenly distributed.

Prepare a 10 ml solution of 10 mg/ml α-amylase in saline with sodium perborate 0.01%.

Step 2: Dissolve in 1 mg of Polyquaternium-1 (Santa Cruise Biochemicals Catalog #sc-476677). Dissolve 40 mg of calcium chloride and 56 mg of sodium chloride into the solution.

### 2.1.4 Composition Development—α-Amylase Formulation+Stabilized Oxvchloro Complex (Composition 16):

Protocol for preparing a 10 ml solution of 10 mg/ml α-amylase in saline with stabilized oxychloro complex:

Add 100 mg of α-amylase from Aspergillus oryzae (Sigma-Aldrich Catalog #10065) to 10 ml of Blink Tears Lubricating Eye Drops in a flask, which already comprise a suitable amount of stabilized oxychloro complex. Mix until α-amylase is dissolved and evenly distributed.

### 2.1.5 Composition Development—α-Amylase Formulation+GenAqua/Dequest (Sodium Perborate) (Composition 17):

Protocol for preparing a 2 ml solution of 10 mg/ml α-amylase in saline with GenAqua/Dequest (sodium perborate) 0.01%:

Step 1: Add 100 mg of α-amylase from Aspergillus oryzae (Sigma-Aldrich Catalog #10065) to DI in a flask. Mix until α-amylase is dissolved and evenly distributed.

Step 2: Dissolve 1 mg of sodium perborate into the solution.

Step 3: Dissolve in 30 mg of calcium chloride and 60 mg of sodium chloride.

### 2.1.6 Composition Development—α-Amylase Formulation+Edetate Disodium 0.1% and Sorbic Acid 0.1% (Composition 18):

Protocol for preparing a sodium hypochlorite solution:

Step 1: Prepare 10 ml 5% bleach (Sodium hypochlorite) solution.

Step 2: Add 6.66 ml of commercially available bleach 7.5% Sodium hypochlorite to a 10 ml flask and fill to 10 ml with DI water.

2.2 Composition Assessment—Well Plate Set Up for In-Vitro Testing Compatibility of α-Amylase from Aspergillus Oryzae with Ophthalmic Preservatives

Using the protocol listed in Example 1 (1.2.1), a plate is prepared for plating Staph. Aureus for preservative compatibility protocol testing. Briefly, S. Aureus is plated as described in Example 1 for rolls 1-10, 1-8 leaving rows 11-12 for DI water for spectrophotometry comparison. The well plates are incubated for 18 hrs at 37° C. before being assayed for biofilm formation.

### 2.3 Composition Testing

### 1.3.1 Test Compositions Day 1

Carefully tilt plate and pipet all liquid from the wells by inserting the pipet tips in the bottom corner of the wells to remove non-adherent cells without disturbing the biofilm grown on the bottom of the well.

Start a timer for 10 minutes when the first row is treated using an 8-channel pipette. Each row should be treated every 30 seconds making sure to gently release the composition in the corner of the well to not disturb the biofilm mechanically.

For Row 1: add 50 μl of 10 mg/ml α-amylase with cofactors to row 1.

For Row 2: add 50 μl of 10 mg/ml α-amylase with benzalkonium chloride 0.01% to row 2.

For Row 3: add 50 μl of 10 mg/ml α-amylase with Polyquad 0.001% to row 3.

For Row 4: add 50 μl of 10 mg/ml α-amylase with sodium perborate 0.01% to row 4.

For Row 5: add 50 μl of 10 mg/ml α-amylase with stabilized oxychloro complex to row 5.

For Row 6: add 50 μl of 7.5% bleach (Sodium hypochlorite) solution to row 6.

For Row 7: add 50 μl DI water to row 7.

Incubate plate for the remaining 10 minutes at 37° C.

### 2.4 Assessment of Biofilm Reduction

After 10 min all fluid should be removed row by row every 30 seconds starting with Row 1. The fluid must be removed with gentle pipetting, with the plate at an angle, with the pipette in the corner to remove non-adherent cells without disturbing the biofilm grown on the bottom of the well.

Add 50 μl of 0.1% crystal violet (CV) to stain adherent cells for all wells.

Carefully rinse all wells by submerging the plate in a DI water bath to remove all remaining crystal violet.

Dry the well plate and measure the optical density of the biofilms using a plate reader at 600 nm.

Once all measurements have been taken the well plate should be transferred to a bleach solution for sanitization and disposed of in a biological waste bin.

### Example 3. Biofilm Formation

Overview of Protocol for Human Conjunctival Epithelial Cells and Staphylococcus aureus Co-Culture Study.

Human Conjunctival Epithelial Cells (HConEpiC) from ScienCell Research Laboratories Catalog #6630 are grown as a monolayer on a poly-L-lysine-coated 24 well plate using Corneal Epithelial Cell Medium.

### 3.1 Initiating the Culture:

ScienCell primary cells are cultured in a 37° C., 5% CO₂ incubator as follows:

1. Prepare a poly-L-lysine-coated culture vessel (2 μg/cm², T-75 flask is recommended). To obtain a 2 μg/cm² poly-L-lysine-coated culture vessel, add 10 ml of sterile water to a T-75 flask and then add 15 l of poly-L-lysine stock solution (10 mg/ml, Cat. #0413). Leave the vessel in a 37° C. incubator overnight (or for a minimum of one hour).

2. Prepare complete medium. Decontaminate the external surfaces of medium bottle and medium supplement tubes with 70% ethanol and transfer them to a sterile field. Aseptically transfer supplement to the basal medium with a pipette. Rinse the supplement tube with medium to recover the entire volume.

3. Rinse the poly-L-lysine-coated vessel twice with sterile water and then add 20 ml of complete medium. Leave the vessel in the sterile field and proceed to thaw the cryopreserved cells.

4. Place the frozen vial in a 37° C. water bath. Hold and rotate the vial gently until the contents completely thaw. Promptly remove the vial from the water bath, wipe it down with 70% ethanol, and transfer it to the sterile field.

5. Carefully remove the cap without touching the interior threads. Gently resuspend and dispense the contents of the vial into the equilibrated, poly-L-lysine-coated culture vessel.

Note: Dilution and centrifugation of cells after thawing is not recommended since these actions can be harmful to the cells. It is also important to plate cells in conditions that favor cell attachment, e.g., in poly-L-lysine-coated culture vessels to promote cell attachment.

6. Replace the cap or lid of the culture vessel and gently rock the vessel to distribute the cells evenly. Loosen cap, if necessary, to allow gas exchange.

7. Return the culture vessel to the incubator.

8. Do not disturb the culture for at least 16 hours after initiation. Refresh culture medium the next day to remove residual DMSO and unattached cells.

### 3.2 Maintaining the Culture:

1. Refresh supplemented culture medium the next morning after establishing a culture from cryopreserved cells.

2. Change the medium every three days thereafter, until the culture is approximately 70% confluent.

3. Once the culture reaches 70% confluency, change medium every other day until the culture is approximately 90% confluent.

### 3.3 Subculturing:

1. Subculture when the culture reaches 90% confluency.

2. Prepare poly-L-lysine-coated culture vessels (2 μg/cm²) one day before subculture.

3. Warm complete medium, trypsin/EDTA solution, 0.05% (T/E, Cat. #0183), T/E neutralization solution (TNS, Cat. #0113), and DPBS (Ca++- and Mg++-free, Cat. #0303) to room temperature. We do not recommend warming reagents and medium in a 37° C. water bath prior to use.

4. Rinse the Cells with DPBS.

5. Add 10 ml 0.05% T/E solution (Cat. #0183) into flask (in the case of a T-75 flask). Gently rock the flask to ensure complete coverage of cells by T/E solution. Use a microscope to monitor the change in cell morphology.

Note: Recommend using ScienCell 0.05% TIE solution which is optimized to minimize cell damage due to over trypsinization, although other solutions can be used. If 0.25% TIE solution (Cat. #0103) is used, then 8 ml of DPBS and 2 ml of 0.25% TIE solution should be used.

6. During incubation, prepare a 50 ml conical centrifuge tube with 5 ml of fetal bovine serum (FBS, Cat. #0500).

7. Once the cells completely round up, transfer T/E solution from the flask to the 50 ml centrifuge tube (a small percent of cells may detach) and incubate the flask at 37° C. for 2-3 minutes (no solution in the flask at this time).

8. At the end of incubation, tap the side of the flask to dislodge cells from the surface. Check under a microscope to make sure that all cells detach.

9. Add 5 ml of TNS solution to the flask and transfer detached cells to the 50 ml centrifuge tube. Rinse the flask with another 5 ml of TNS to collect the residual cells.

10. Examine the flask under a microscope for a successful cell harvest by looking at the number of cells being left behind; there should be less than 5%.

11. Centrifuge the 50 ml centrifuge tube at 1000 rpm for 5 minutes. Gently resuspend cells in culture medium.

12. Count and plate cells in a new poly-L-lysine-coated culture vessel with the recommended cell density. A seeding density of 5,000 cells/cm² is recommended.

### 3.4 Co-Culture Development

After the establishment of a monolayer of human conjunctival epithelial cells, a layer of Staphylococcus aureus subsp. aureus Rosenbach (ATCC 29213) will be established on top of the epithelial cells.

1. Add 650 μl of Staphylococcus aureus subsp. aureus Rosenbach (ATCC 29213) in Tryptic Soy Broth to the TSB citrate media (TSBC) diluted to A600 of 0.01 (~1×10\hat{ }8 CFU/ml) into each well in rows A-C leaving row D untreated.

2. Cover the well plate and incubate for 4 hrs at 37° C.

### 3.5 Testing

Compositions of α-amylase, preferentially the compositions that have demonstrated the ability to remove biofilms in cell culture as described in Example 1, are tested in this co-culture to study to characterize their potential toxicity to the underlying monolayer of human conjunctival epithelial cells. The hypothesis is that with treatment and elimination of the insulting S. aureus, the inflammatory response from the epithelial cells will revert to normal at the mRNA and protein level.

1 ml of each test composition is added to each well of row A.

Row B will is tested with I ml of a negative control (saline).

Row C is tested with 1 ml of a positive control (gentamicin).

Test compositions should be incubated for 10 min at 37° C.

All layers should be washed three times prior to lysing. Plates are gently lysed with 1 ml trypsin to remove cells. Lysed cells are stained with trypan blue and evaluated under a microscope for cell counting. The presence of blue cells indicates cell death. Samples are evaluated for cell number and visibility curves. Samples are also collected and frozen at pre- and post-challenge for mRNA, DNA, and protein analysis for future expression analysis.

### Example 4. Treating Antibiotics+α-Amylase on S. Aureus Biofilms

S. Aureus produces extracellular capsular polysaccharides which link to create biofilms. Aspergillus oryzae α-amylase (E.C. 3.2.1.1) catalyzes endohydrolysis of 1,4-alpha-D-glucosidic linkages in polysaccharides containing three or more 1,4-alpha-linked D-glucose units. α-amylase does not have antibacterial properties, but it can inhibit and reduce S. Aureus biofilms by breaking the extracellular polysaccharides bonds of the biofilm. Antibiotics have been shown to have some effect in reducing biofilms, but are less effective on established biofilms. Further, prolonged use of antibiotics can lead to antibiotic resistant bacteria. α-amylase does not have antibacterial properties and does not have a known risk of creating antibiotic resistant bacteria.

The external application of α-amylase described in this example has never been used to reduce or prevent biofilm formation on the eyelid. This experiment aims to characterize the use of α-amylase and α-amylase compositions with other antibacterial or antimicrobial agents on S. Aureus biofilms grown in well plates for 24 hours. It is hypothesized that the addition of a-amylase will improve the biofilm removal of all compounds.

This study describes the testing of compositions of 0.01% Hypochlorous Acid solution with and without α-amylase from Aspergillus oryzae on biofilms formed in-vitro. This study also describes the testing of compositions of common ophthalmic antibiotics with and without α-amylase from Aspergillus oryzae. These antibiotics include: Erythromycin, Moxifloxacin, and Gentamicin Sulfate. This study uses sterile saline as a negative control and a 5% bleach solution as a positive control.

### 4.1 Composition Development—α-Amylase Formulation in Saline:

α-amylase requires calcium ions to function. Saline solutions are prepared with 0.4% calcium chloride and 0.56% sodium chloride. 0.4% calcium chloride is added to α-amylase solutions to ensure calcium ions present.

Step 1: Prepare a 10 ml solution of 10 mg/ml α-amylase with cofactors. Add 100 mg of α-amylase from Aspergillus oryzae (Sigma-Aldrich Catalog #10065) to 10 ml sterile DI Water in a flask. Mix until α-amylase is dissolved and evenly distributed.

Step 2: Dissolve 40 mg of calcium chloride and 56 mg of sodium chloride into the solution. Mix until all ingredients are evenly distributed.

### 4.1.2 Composition Development—α-Amylase in 0.01% Hypochlorous Acid Solution

Protocol for preparing a 2 ml solution of 10 mg/ml α-amylase in 0.01% Hypochlorous Acid solution:

Step 1: Add 100 mg of α-amylase from Aspergillus oryzae (Sigma-Aldrich Catalog #10065) to 10 ml of 0.01% Hypochlorous Acid (Avenova OTC Antimicrobial Spray Solution) in saline in a flask. Mix until α-amylase is dissolved and evenly distributed.

Step 2: dissolve 40 mg of calcium chloride into the solution. Mix until α-amylase is dissolved and evenly distributed.

### 4.1.3 Composition Development—α-Amylase in Ofloxacin Solution

Protocol for preparing a 2 ml of 10 mg/ml α-amylase in an Ofloxacinophthalmic solution 0.3%:

Briefly: add 20 mg of α-amylase from Aspergillus oryzae (Sigma-Aldrich Catalog #10065) to 1 ml of Erythromycin ophthalmic solution 0.5% in a flask and mix until evenly distributed. Dissolve 8 mg of calcium chloride into the solution.

### 4.1.4 Composition Development—α-Amylase in 0.01% Moxifloxacin Solution

Protocol for preparing a 1 ml of 10 mg/ml α-amylase in a Moxifloxacin ophthalmic solution 0.5%:

Briefly: add 10 mg of α-amylase from Aspergillus oryzae (Sigma-Aldrich Catalog #10065) to 1 ml of Moxifloxacin ophthalmic solution 0.5% in a flask and mix until evenly distributed. Dissolve 3 mg of calcium chloride into the solution.

### 4.1.5 Composition Development—α-Amylase in Gentamicin Sulfate

Protocol for preparing a 2 ml of 10 mg/ml α-amylase in a Gentamicin Sulfate ophthalmic solution 0.5%:

Briefly: add 20 mg of α-amylase from Aspergillus oryzae (Sigma-Aldrich Catalog #10065) to 1 ml of Gentamicin Sulfate ophthalmic solution 0.5% in a flask and mix until evenly distributed. Dissolve 8 mg of calcium chloride into the solution.

### 4.1.6 Composition Development—α-Amylase in Bleach

Protocol for preparing a 10 ml 5% bleach (Sodium hypochlorite) solution:

Briefly: add 6.66 ml of commercially available bleach 7.5% Sodium hypochlorite to a 10 ml flask and fill to 10 ml with DI water.

### 4.2 Well Plate Setup

Using the protocol listed in Example 1 (1.2.1), a plate is prepared for plating Staph. Aureus for preservative compatibility protocol testing. Briefly, S. Aureus is plated as described in Example 1, in all wells of a 96 tissue culture treated well plate. The well plate should incubate for 18 hrs at 37° C. before being assayed for biofilm formation.

### 4.3 Test Compositions

Carefully tilt plate and pipet all liquid from the wells by inserting the pipet tips in the bottom corner of the wells to remove non-adherent cells without disturbing the biofilm grown on the bottom of the well.

Start a timer for 10 minutes when the first row is treated using an 8-channel pipette. Each row should be treated every 30 seconds making sure to gently release the composition in the corner of the well to not disturb the biofilm mechanically.

For Row 1: add 50 μl of 10 mg/ml α-amylase in co-factor solution to all wells.

For Row 2: add 50 μl of 10 mg/ml α-amylase in 0.01% Hypochlorous Acid solution to all wells.

For Row 3: add 50 μl of 0.01% Hypochlorous Acid (Avenova OTC Antimicrobial Spray Solution) in saline to all wells.

For Row 4: add 50 μl of 10 mg/ml α-amylase in an Ofloxacin ophthalmic solution 0.3% to all wells.

For Row 5: add 50 μl of Ofloxacin ophthalmic solution 0.3% to all wells.

For Row 6: add 50 μl of 10 mg/ml α-amylase in a Gentamicin Sulfate ophthalmic solution 0.5% to all wells.

For Row 7: add 50p Gentamicin Sulfate ophthalmic solution 0.5% to all wells.

For Row 8: add 50 μl of 5% bleach (Sodium hypochlorite) solution to all wells.

For Row 9: add 50 μl DI water to all wells.

Do not treat Rows 10-12. Incubate plate for the remaining 10 minutes at 37 C.

### 4.4. Test for Biofilm Reduction

After 10 min all fluid should be removed row by row every 30 seconds starting with Row 1. The fluid must be removed with gentle pipetting, with the plate at an angle, with the pipette in the corner to remove non-adherent cells without disturbing the biofilm grown on the bottom of the well.

Add 50 μl of 0.1% crystal violet (CV) to stain adherent cells for all wells.

Carefully rinse all wells by submerging the plate in a DI water bath to remove all remaining crystal violet.

Dry the well plate and measure the optical density of the biofilms using a plate reader at 600 nm. Once all measurements have been taken the well plate should be transferred to a bleach solution for sanitizing and disposed of in a biological waste bin.

### Example 5. Ranges of Cofactors+α-Amylase Treatment on S. Aureus Biofilms

This protocol is designed to evaluate optimal ranges of the cofactor calcium for a-amylase from Aspergillus oryzae when used to remove S. Aureus biofilms.

Ca²⁺ has been identified as a necessary cofactor for α-amylase, however prior research into α-amylase on S. Aureus biofilms has not introduced Ca²⁺ to the compositions to remove or inhibit biofilm growth in cell culture. α-amylase has two binding sites for Ca ions. 1 mg of a-amylase contains 1.075*10\hat{ }16 enzymes, and the disclosure provides that it may take at least 0.0039 mg of CaCl₂ to provide two Calcium ions per enzyme. It is not clear how much Ca²⁺ is needed to provide optimal functionality for removing biofilms and some literature suggests that excess calcium could have a negative effect. Although some electrolytes, including Ca²⁺, are present naturally in the eye, this study is designed to characterize the effectiveness of α-amylase from Aspergillus oryzae in removing S. Aureus biofilms in cell culture at different concentrations of the Ca²⁺ cofactor.

Normal Electrolyte Concentration in Human Tears (mMol/Liter)

TABLE 16

| Electrolyte | mMol/Liter |
|---|---|
| Sodium (Na) | 132 |
| Potassium | 24 |
| HCO₂ | 32.8 |
| Ca | 0.8 |
| Mg | 0.61 |

### 5.1 Composition Development

5.1.1—Prepare a 10 ml Solution of 10 mg/Ml α-Amylase in DI Water

Add 100 mg of α-amylase from Aspergillus oryzae (Sigma-Aldrich Catalog #10065) to 2 ml sterile DI water in a flask. Mix until α-amylase is dissolved and evenly distributed.

5.1.2—Prepare a 10 ml Solution of 10 mg/Ml α-Amylase in DI Water with 0.05% CaCl₂) 0.85% NaCl.

Add 100 mg of α-amylase from Aspergillus oryzae (Sigma-Aldrich Catalog #10065) to 10 ml sterile DI water in a flask. Mix until α-amylase is dissolved and evenly distributed. Add 5 mg of CaCl₂. Add 85 mg of NaCl. Mix until dissolved.

5.1.3—Prepare a 10 ml Solution of 10 mg/Ml α-Amylase in DI Water with 0.1% CaCl₂) 0.8% NaCl.

Add 100 mg of α-amylase from Aspergillus oryzae (Sigma-Aldrich Catalog #10065) to 10 ml sterile DI water in a flask. Mix until α-amylase is dissolved and evenly distributed. Add 10 mg of CaCl₂. Add 80 mg of NaCl. Mix until dissolved.

5.1.4—Prepare a 10 ml Solution of 10 mg/Ml α-Amylase in DI Water with 0.2% CaCl₂) 0.7% NaCl.

Add 100 mg of α-amylase from Aspergillus oryzae (Sigma-Aldrich Catalog #10065) to 10 ml sterile DI water in a flask. Mix until α-amylase is dissolved and evenly distributed. Add 20 mg of CaCl₂. Add 70 mg of NaCl. Mix until dissolved.

5.1.5—Prepare a 10 ml Solution of 10 mg/Ml α-Amylase in DI Water with 0.3% CaCl₂ 0.6% NaCl.

Add 100 mg of α-amylase from Aspergillus oryzae (Sigma-Aldrich Catalog #10065) to 10 ml sterile DI water in a flask. Mix until α-amylase is dissolved and evenly distributed. Add 30 mg of CaCl₂. Add 60 mg of NaCl. Mix until dissolved.

5.1.6—Prepare a 10 ml Solution of 10 mg/Ml α-Amylase in DI Water with 0.4% CaCl₂ 0.5% NaCl.

Add 100 mg of α-amylase from Aspergillus oryzae (Sigma-Aldrich Catalog #10065) to 10 ml sterile DI water in a flask. Mix until α-amylase is dissolved and evenly distributed. Add 40 mg of CaCl₂. Add 50 mg of NaCl. Mix until dissolved.

5.1.7—Prepare a 10 ml Solution of 10 mg/Ml α-Amylase in DI Water with 0.5% CaCl₂ 0.4% NaCl.

Add 100 mg of α-amylase from Aspergillus oryzae (Sigma-Aldrich Catalog #10065) to 10 ml sterile DI water in a flask. Mix until α-amylase is dissolved and evenly distributed. Add 50 mg of CaCl₂. Add 40 mg of NaCl. Mix until dissolved

5.1.8—Prepare a 10 ml Solution of 10 mg/Ml α-Amylase in DI Water with 0.6% CaCl₂ 0.3% NaCl

Add 100 mg of α-amylase from Aspergillus oryzae (Sigma-Aldrich Catalog #10065) to 10 ml sterile DI water in a flask. Mix until α-amylase is dissolved and evenly distributed. Add 60 mg of CaCl₂. Add 30 mg of NaCl. Mix until dissolved.

5.1.9—Prepare a 10 ml Solution of 10 mg/Ml α-Amylase in DI Water with 0.8% CaCl₂ 0.1% NaCl

Add 100 mg of α-amylase from Aspergillus oryzae (Sigma-Aldrich Catalog #10065) to 10 ml sterile DI water in a flask. Mix until α-amylase is dissolved and evenly distributed. Add 80 mg of CaCl₂. Add 10 mg of NaCl. Mix until dissolved.

### 5.2 Test Compositions

Carefully tilt plate and pipet all liquid from the wells by inserting the pipet tips in the bottom corner of the wells to remove non-adherent cells without disturbing the biofilm grown on the bottom of the well.

Start a timer for 10 minutes when the first row is treated using an 8-channel pipette. Each row should be treated every 30 seconds making sure to gently release the composition in the corner of the well to not disturb the biofilm mechanically.

For Row 1: Add 50 μl of 10 mg/ml α-amylase in DI water to row 1.

For Row 2: Add 50 μl solution of 10 mg/ml α-amylase in DI water with 0.05% CaCl₂ 0.85% NaCl to row 2.

For Row 3: Add 50 μl solution of 10 mg/ml α-amylase in DI water with 0.1% CaCl₂ 0.8% NaCl to row 3.

For Row 4: Add 50 μl solution of 10 mg/ml α-amylase in DI water with 0.2% CaCl₂ 0.7% NaCl to row 4.

For Row 5: Add 50 μl solution of 10 mg/ml α-amylase in DI water with 0.3% CaCl₂ 0.6% NaCl to row 5.

For Row 6: Add 50 μl solution of 10 mg/ml α-amylase in DI water with 0.4% CaCl₂ 0.5% NaCl to row 6.

For Row 7: Add 50 μl solution of 10 mg/ml α-amylase in DI water with 0.5% CaCl₂ 0.4% NaCl to row 7.

For Row 8: Add 50 μl solution of 10 mg/ml α-amylase in DI water with 0.6% CaCl₂ 0.3% NaCl to row 8.

For Row 9: Add 50 μl solution of 10 mg/ml α-amylase in DI water with 0.8% CaCl₂ 0.1% NaCl to row 9.

For Row 10: Add 50 μl of 7.5% bleach (Sodium hypochlorite) solution to row 10.

For Row 11: Add 50 μl DI water to row 11.

Do not treat Row 12. The plate should be placed in the incubator at 37 C for the remainder of the 10 min period.

### 5.3 Test for Biofilm

After 10 min all fluid should be removed row by row every 30 seconds starting with Row 1. The fluid must be removed with gentle pipetting, with the plate at an angle, with the pipette in the corner to remove non-adherent cells without disturbing the biofilm grown on the bottom of the well.

Add 50 μl of 0.1% crystal violet (CV) to stain adherent cells for all wells.

Carefully rinse all wells by submerging the plate in a DI water bath to remove all remaining crystal violet. Dry the well plate and measure the optical density of the biofilms using a plate reader at 600 nm.

Once all measurements have been taken the well plate should be transferred to a bleach solution for sanitization and disposed of in a biological waste bin.

### Example 6. α-Amylase Compositions Comprising Calcium Cofactor (CaCl₂)) in Concentrations Ranging from 0.4% CaCl₂) to 0.8% CaCl₂)Volume

The experiments below summarize outcomes observed with different compositions comprising various additives to α-amylase from Aspergillus oryzae to determine if any such additives enhance, do not effect, or inhibit the enzyme's ability to remove biofilms grown in cell culture. The remaining biofilm density was assessed using a cell plate reader at 600 nm called an optical density 600 (OD₆₀₀) measurement.

### Example 6.1. α-amylase Compositions Comprising Calcium Cofactor (CaCl₂)

### Calcium Cofactor Optimization:

Calcium has been identified as a necessary cofactor for α-amylase, however prior research into α-amylase on S. Aureus biofilms have not introduced calcium to the compositions to remove or inhibit biofilm growth in cell culture. This study was initiated to determine how much calcium is needed to provide optimal functionality for removing biofilms. Some literature suggests that excess calcium could have a negative effect. It was thus important to consider concentration ranges of Ca²⁺ that support α-amylase activity without inhibiting it.

### General Methods:

Staphylococcus aureus biofilms were grown in 96 well (Corning Falcon) cell culture treated plates using a TSB broth supplemented with yeast extract, glucose, and sodium citrate to induce biofilm formation and incubated for 18 hours.

Non-adherent cells were removed and the biofilms were treated with different solutions to test the ability to break down the Staph. aureus biofilms. Solutions with α-amylase were produced at 10 mg/mL using α-amylase from Aspergillus oryzae (Sigma-Aldrich Catalog #10065) ~30 U/mg.

After 10 minutes of incubation non-adherent cells were removed and the residual biofilms were stained with crystal violet. The density of the remaining biofilm was measured at 600 nm using a Byonoy Absorbance 96 μlate reader.

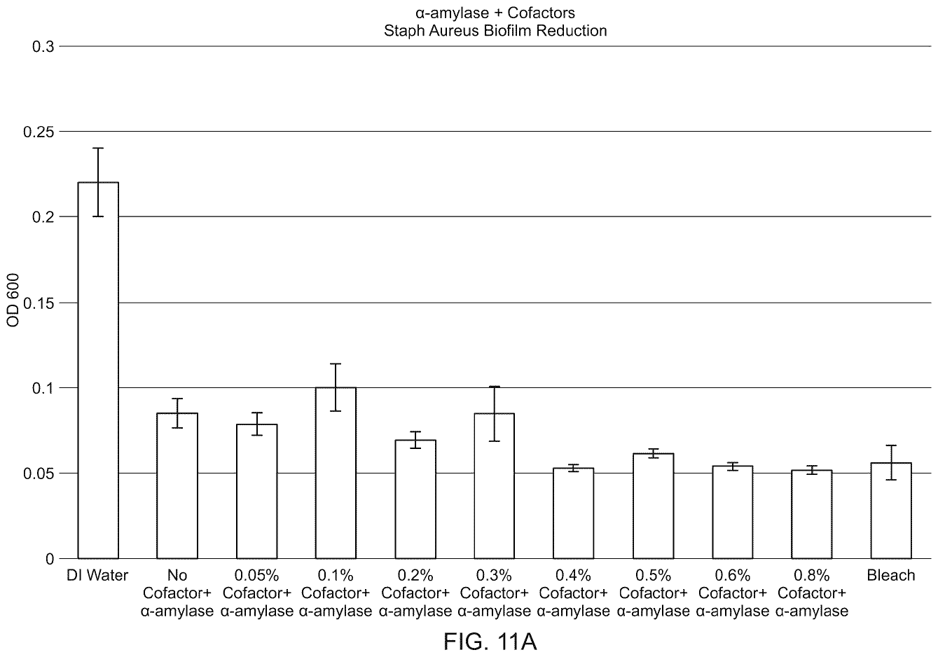

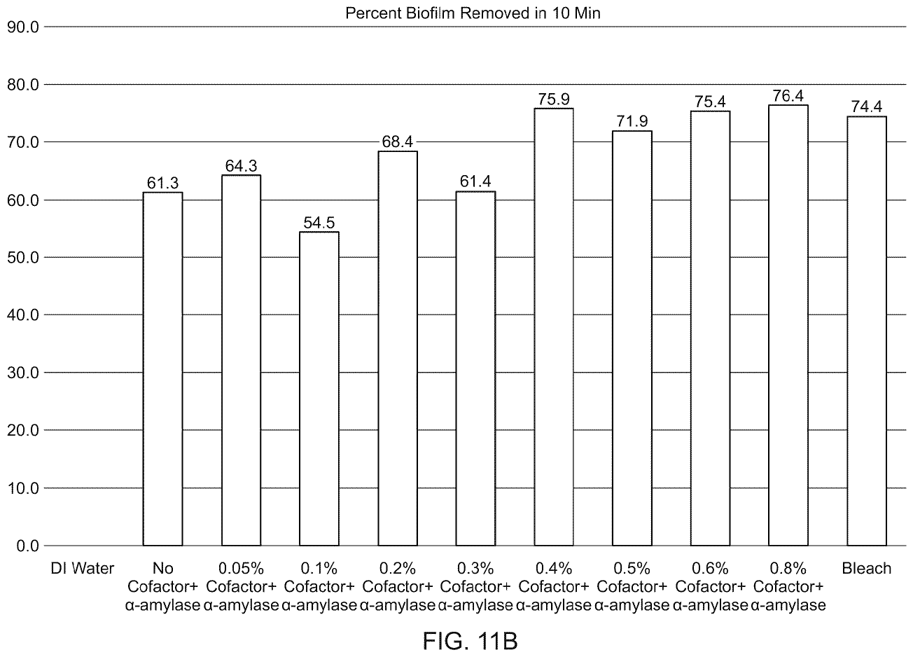

FIG. 11A is a chart illustrating the results of the experiment described above. The optical density of the biofilm is shown on the Y-axis. The X-axis illustrates various concentrations ofthe CaCl₂ co-factors and two controls: deionized water (negative control) and bleach (positive control for removal of biofilm). The concentrations tested include 0.0500 CaCl₂ cofactor, 0.10 CaCl₂ cofactor, 0.2% CaCl₂ cofactor, 0.3% CaCl₂ cofactor, 0.4 CaCl₂ cofactor, 0.5 CaCl₂ cofactor, 0.6% CaCl₂ cofactor, or 0.8% CaCl₂ cofactor,

The results of the Tukey's multiple comparisons test analysis, summarized in the Table below:

TABLE 17

| | Mean | 95.00% CI | Below | | Adjusted |
|---|---|---|---|---|---|
| Tukey's multiple comparisons test | Diff. | of diff. | threshold? | Summary | P Value |
| DI Water vs. No Cofactor + α- | 0.135 | 0.08803 to | Yes | **** | <0.0001 |
| amylase | | 0.1820 | | | |
| DI Water vs. 0.05% Cofactor + α- | 0.1415 | 0.09453 to | Yes | **** | <0.0001 |
| amylase | | 0.1885 | | | |
| DI Water vs. 0.1% Cofactor + α- | 0.1199 | 0.07290 to | Yes | **** | <0.0001 |
| amylase | | 0.1668 | | | |
| DI Water vs. 0.2% Cofactor + α- | 0.1505 | 0.1035 to | Yes | **** | <0.0001 |
| amylase | | 0.1975 | | | |
| DI Water vs. 0.3% Cofactor + α- | 0.1353 | 0.08828 to | Yes | **** | <0.0001 |
| amylase | | 0.1822 | | | |
| DI Water vs. 0.4% Cofactor + α- | 0.167 | 0.1200 to | Yes | **** | <0.0001 |
| amylase | | 0.2140 | | | |
| DI Water vs. 0.5% Cofactor + α- | 0.1584 | 0.1114 to | Yes | **** | <0.0001 |
| amylase | | 0.2053 | | | |
| DI Water vs. 0.6% Cofactor + α- | 0.166 | 0.1190 to | Yes | **** | <0.0001 |
| amylase | | 0.2130 | | | |
| DI Water vs. 0.8% Cofactor + α- | 0.1681 | 0.1212 to | Yes | **** | <0.0001 |
| amylase | | 0.2151 | | | |
| DI Water vs. Bleach | 0.1639 | 0.1169 to | Yes | **** | <0.0001 |
| | | 0.2108 | | | |
| No Cofactor + α-amylase vs. 0.05% | 0.0065 | −0.04047 to | No | ns | >0.9999 |
| Cofactor + α-amylase | | 0.05347 | | | |
| No Cofactor + α-amylase vs. 0.1% | −0.01513 | −0.06210 to | No | ns | 0.9919 |
| Cofactor + α-amylase | | 0.03185 | | | |
| No Cofactor + α-amylase vs. 0.2% | 0.0155 | −0.03147 to | No | ns | 0.9902 |
| Cofactor + α-amylase | | 0.06247 | | | |
| No Cofactor + α-amylase vs. 0.3% | 0.00025 | −0.04672 to | No | ns | >0.9999 |
| Cofactor + α-amylase | | 0.04722 | | | |
| No Cofactor + α-amylase vs. 0.4% | 0.032 | −0.01497 to | No | ns | 0.4711 |
| Cofactor + α-amylase | | 0.07897 | | | |
| No Cofactor + α-amylase vs. 0.5% | 0.02338 | −0.02360 to | No | ns | 0.8548 |
| Cofactor + α-amylase | | 0.07035 | | | |
| No Cofactor + α-amylase vs. 0.6% | 0.031 | −0.01597 to | No | ns | 0.5192 |
| Cofactor + α-amylase | | 0.07797 | | | |
| No Cofactor + α-amylase vs. 0.8% | 0.03313 | −0.01385 to | No | ns | 0.4187 |
| Cofactor + α-amylase | | 0.08010 | | | |
| No Cofactor + α-amylase vs. | 0.02888 | −0.01810 to | No | ns | 0.6227 |
| Bleach | | 0.07585 | | | |
| 0.05% Cofactor + α-amylase vs. | −0.02163 | −0.06860 to | No | ns | 0.906 |
| 0.1% Cofactor + α-amylase | | 0.02535 | | | |
| 0.05% Cofactor + α-amylase vs. | 0.009 | −0.03797 to | No | ns | >0.9999 |
| 0.2% Cofactor + α-amylase | | 0.05597 | | | |
| 0.05% Cofactor + α-amylase vs. | −0.00625 | −0.05322 to | No | ns | >0.9999 |
| 0.3% Cofactor + α-amylase | | 0.04072 | | | |
| 0.05% Cofactor + α-amylase vs. | 0.0255 | −0.02147 to | No | ns | 0.7758 |
| 0.4% Cofactor + α-amylase | | 0.07247 | | | |
| 0.05% Cofactor + α-amylase vs. | 0.01688 | −0.03010 to | No | ns | 0.9816 |
| 0.5% Cofactor + α-amylase | | 0.06385 | | | |
| 0.05% Cofactor + α-amylase vs. | 0.0245 | −0.02247 to | No | ns | 0.8152 |
| 0.6% Cofactor + α-amylase | | 0.07147 | | | |
| 0.05% Cofactor + α-amylase vs. | 0.02663 | −0.02035 to | No | ns | 0.7277 |
| 0.8% Cofactor + α-amylase | | 0.07360 | | | |
| 0.05% Cofactor + α-amylase vs. | 0.02238 | −0.02460 to | No | ns | 0.8857 |
| Bleach | | 0.06935 | | | |
| 0.1% Cofactor + α-amylase vs. | 0.03063 | −0.01635 to | No | ns | 0.5374 |
| 0.2% Cofactor + α-amylase | | 0.07760 | | | |
| 0.1% Cofactor + α-amylase vs. | 0.01538 | −0.03160 to | No | ns | 0.9908 |
| 0.3% Cofactor + α-amylase | | 0.06235 | | | |
| 0.1% Cofactor + α-amylase vs. | 0.04713 | 0.0001542 to | Yes | * | 0.0485 |
| 0.4% Cofactor + α-amylase | | 0.09410 | | | |
| 0.1% Cofactor + α-amylase vs. | 0.0385 | −0.008471 to | No | ns | 0.2109 |
| 0.5% Cofactor + α-amylase | | 0.08547 | | | |
| 0.1% Cofactor + α-amylase vs. | 0.04613 | −0.0008458 to | No | ns | 0.0587 |
| 0.6% Cofactor + α-amylase | | 0.09310 | | | |
| 0.1% Cofactor + α-amylase vs. | 0.04825 | 0.001279 to | Yes | * | 0.0389 |
| 0.8% Cofactor + α-amylase | | 0.09522 | | | |
| 0.1% Cofactor + α-amylase vs. | 0.044 | −0.002971 to | No | ns | 0.0866 |
| Bleach | | 0.09097 | | | |
| 0.2% Cofactor + α-amylase vs. 0.3% | −0.01525 | −0.06222 to | No | ns | 0.9914 |
| Cofactor + α-amylase | | 0.03172 | | | |
| 0.2% Cofactor + α-amylase vs. 0.4% | 0.0165 | −0.03047 to | No | ns | 0.9844 |
| Cofactor + α-amylase | | 0.06347 | | | |
| 0.2% Cofactor + α-amylase vs. 0.5% | 0.007875 | −0.03910 to | No | ns | >0.9999 |
| Cofactor + α-amylase | | 0.05485 | | | |
| 0.2% Cofactor + α-amylase vs. 0.6% | 0.0155 | −0.03147 to | No | ns | 0.9902 |
| Cofactor + α-amylase | | 0.06247 | | | |
| 0.2% Cofactor + α-amylase vs. 0.8% | 0.01763 | −0.02935 to | No | ns | 0.9749 |
| Cofactor + α-amylase | | 0.06460 | | | |
| 0.2% Cofactor + α-amylase vs. | 0.01338 | −0.03360 to | No | ns | 0.997 |
| Bleach | | 0.06035 | | | |
| 0.3% Cofactor + α-amylase vs. | 0.03175 | −0.01522 to | No | ns | 0.4831 |
| 0.4% Cofactor + α-amylase | | 0.07872 | | | |
| 0.3% Cofactor + α-amylase vs. | 0.02313 | −0.02385 to | No | ns | 0.863 |
| 0.5% Cofactor + α-amylase | | 0.07010 | | | |
| 0.3% Cofactor + α-amylase vs. | 0.03075 | −0.01622 to | No | ns | 0.5313 |
| 0.6% Cofactor + α-amylase | | 0.07772 | | | |
| 0.3% Cofactor + α-amylase vs. | 0.03288 | −0.01410 to | No | ns | 0.4302 |
| 0.8% Cofactor + α-amylase | | 0.07985 | | | |
| 0.3% Cofactor + α-amylase vs. | 0.02863 | −0.01835 to | No | ns | 0.6347 |
| Bleach | | 0.07560 | | | |
| 0.4% Cofactor + α-amylase vs. | −0.008625 | −0.05560 to | No | ns | >0.9999 |
| 0.5% Cofactor + α-amylase | | 0.03835 | | | |
| 0.4% Cofactor + α-amylase vs. | −0.001 | −0.04797 to | No | ns | >0.9999 |
| 0.6% Cofactor + α-amylase | | 0.04597 | | | |
| 0.4% Cofactor + α-amylase vs. | 0.001125 | −0.04585 to | No | ns | >0.9999 |
| 0.8% Cofactor + α-amylase | | 0.04810 | | | |
| 0.4% Cofactor + α-amylase vs. | −0.003125 | −0.05010 to | No | ns | >0.9999 |
| Bleach | | 0.04385 | | | |
| 0.5% Cofactor + α-amylase vs. | 0.007625 | −0.03935 to | No | ns | >0.9999 |
| 0.6% Cofactor + α-amylase | | 0.05460 | | | |
| 0.5% Cofactor + α-amylase vs. | 0.00975 | −0.03722 to | No | ns | 0.9998 |
| 0.8% Cofactor + α-amylase | | 0.05672 | | | |
| 0.5% Cofactor + α-amylase vs. | 0.0055 | −0.04147 to | No | ns | >0.9999 |
| Bleach | | 0.05247 | | | |
| 0.6% Cofactor + α-amylase vs. | 0.002125 | −0.04485 to | No | ns | >0.9999 |
| 0.8% Cofactor + α-amylase | | 0.04910 | | | |
| 0.6% Cofactor + α-amylase vs. | −0.002125 | −0.04910 to | No | ns | >0.9999 |
| Bleach | | 0.04485 | | | |
| 0.8% Cofactor + α-amylase vs. | −0.00425 | −0.05122 to | No | ns | >0.9999 |
| Bleach | | 0.04272 | | | |

A reduction in biofilm density indicates removal of the biofilm by the enzyme. The results suggest that concentrations of CaCl₂ from 0.4% to 0.8% consistently remove the biofilm. See FIG. 11A. FIG. 11B is a chart quantifying the percent of biofilm removed in 10 min by each of the tested compositions. Bleach is used as a positive control in FIG. 11B.

### Example 7. Ophthalmic Preservatives

In the configurations where the composition is delivered in individual sterile packaging, sterile filtering and packaging can be used to avoid the addition of preservatives. In cases where the composition is delivered in a multidose package a preservative may be present. This example describes general preservatives that were tested and found not to inhibit enzyme effectiveness:

### General Methods:

Staphylococcus aureus biofilms were grown in 96 well (Crnming Falcon) cell culture treated plates using a TSB broth supplemented with yeast extract, glucose, and sodium citrate to induce biofilm formation and incubated for 18 hours.

Non-adherent cells were removed and the biofilms were treated with different solutions to test the ability to break down the Staph. aureus biofilms. Solutions with α-amylase were produced at 10 mg/mL using α-amylase from Aspergillus oryzae (Sigma-Aldrich Catalog #10065) ~30 U/mg.

After 10 minutes of incubation non-adherent cells were removed and the residual biofilms were stained with crystal violet. The density of the remaining biofilm was measured at 600 nm using a Byonoy Absorbance 96 μlate reader.

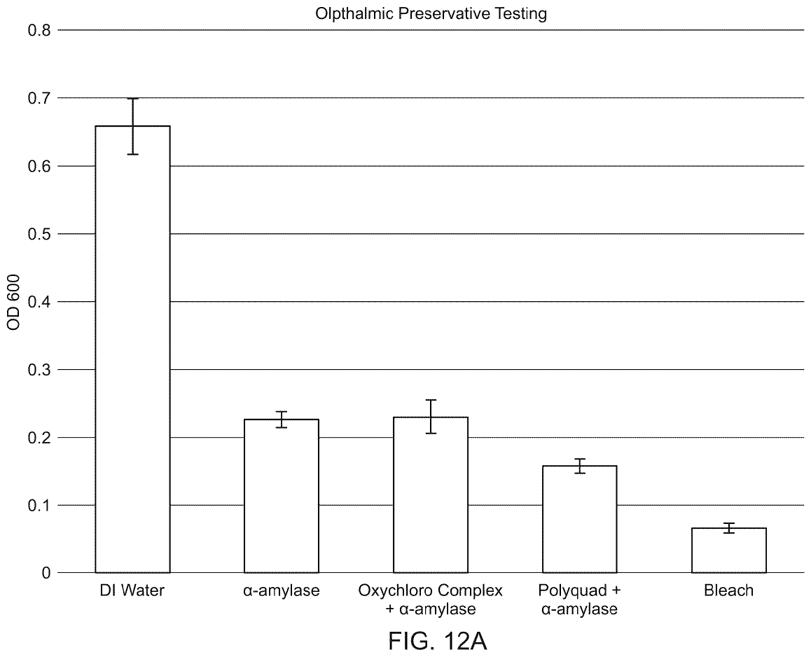

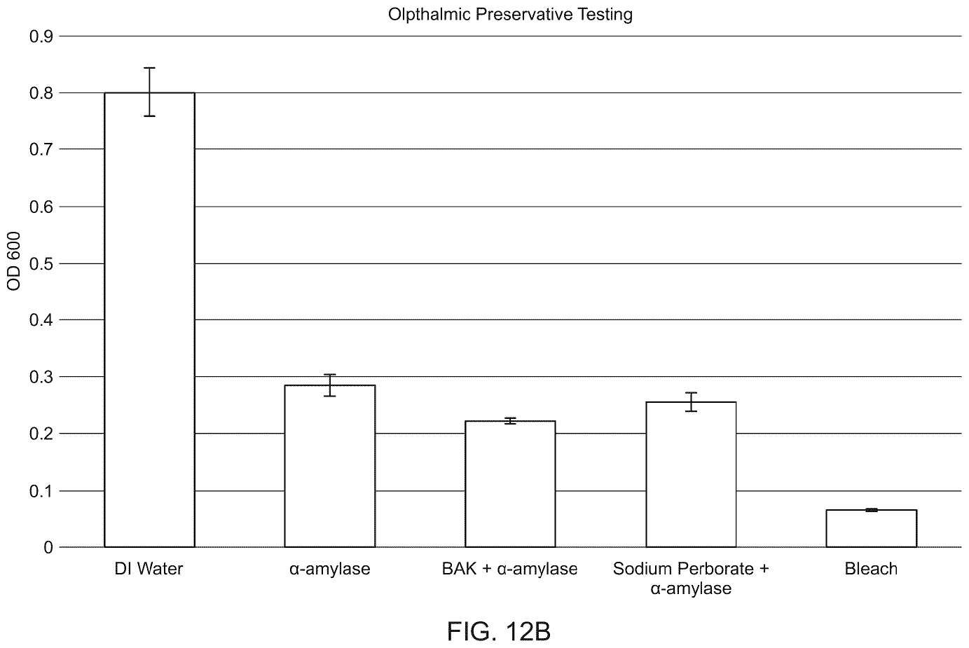

FIG. 12A is chart illustrating the results of the experiment for α-amylase, BAK+α-amylase, sodium perborate+α-amylase, and their comparisons to the negative control (DI water) and positive control (bleach). The preservatives tested in FIG. 12A are: Preservative 1: Purite/OcuPure (stabilized oxychloro complex)−No Reduction in Enzyme Effectiveness; Preservative 2: Polyquad (polyquaternium-1) 0.001%−No Reduction in Enzyme Effectiveness.

The preservatives tested in FIG. 12B are: Preservative 1: BAK (benzalkonium chloride) 0.01% —No Reduction in Enzyme Effectiveness; and Preservative 2 GenAqua/Dequest (sodium perborate)−No Reduction in Enzyme Effectiveness.

The aforementioned tested compositions in the specified concentrations were found not to inhibit enzyme activity.

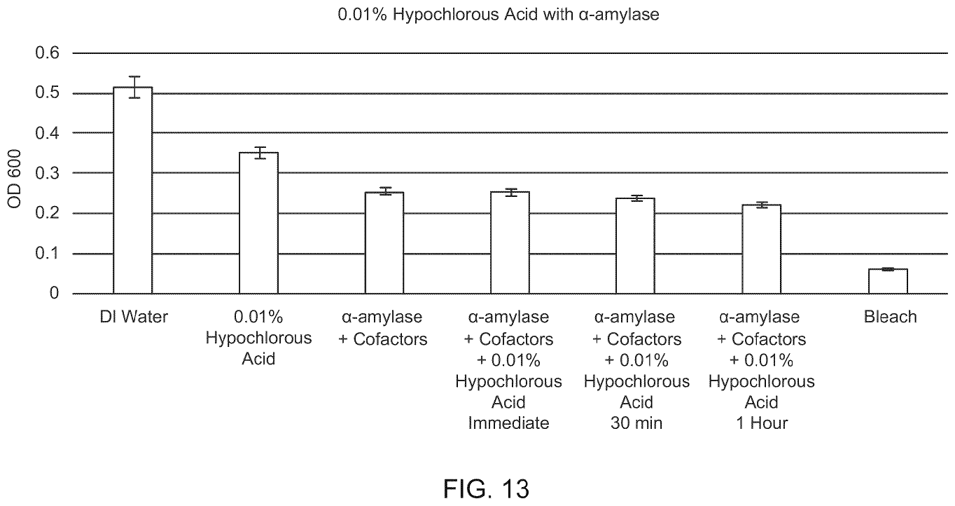

Another preservative for use in treating dry eye conditions tested was 0.01% hypochlorous acid. Dilute hypochlorous acid is used in eyelid cleaners in percentages ranging from 0.01% to 0.02%. In our studies 0.01% hypochlorous acid did not inhibit biofilm removal by the enzyme. There was no statistical difference between biofilm reduction with 0.01% hypochlorous acid and α-amylase plus cofactors mixed immediately, 30 minutes, or 1 hour before treatment. While 0.01% hypochlorous acid did slightly reduce the biofilm, when combined with α-amylase and cofactors there was no additive benefit of increased biofilm removal. See FIG. 13.

### Example 8. Ophthalmic Demulcents

This protocol investigated the efficacy of α-amylase, an enzyme effective in inhibiting and reducing S. Aureus biofilms, in combination with ophthalmic demulcents. These demulcents are used in eye drops to alleviate symptoms of dry eye and could potentially prolong the contact of α-amylase with the biofilm on the eye. Some demulcents could also have a stabilizing effect on the enzyme. The study assessed the impact of various demulcents on the activity of α-amylase.

The following demulcents were contemplated:
(a) Cellulose derivatives: (1) Carboxymethylcellulose sodium, 0.2 to 2.5 percent; (2) Hydroxyethyl cellulose, 0.2 to 2.5 percent; (3) Hypromellose, 0.2 to 2.5 percent; (4) Methylcellulose, 0.2 to 2.5 percent.
(b) Dextran 70, 0.1 percent when used with another polymeric demulcent agent in this section.
(c) Gelatin, 0.01 percent.
(d) Polyols, liquid: (1) Glycerin, 0.2 to 1 percent; (2) Polyethylene glycol 300, 0.2 to 1 percent; (3) Polyethylene glycol 400, 0.2 to 1 percent; (4) Polysorbate 80, 0.2 to 1 percent; (5) Propylene glycol, 0.2 to 1 percent.
(e) Polyvinyl alcohol, 0.1 to 4 percent.
(f) Povidone, 0.1 to 2 percent.

All tested solutions were prepared with 10 mg/mL α-amylase from Aspergillus oryzae (Sigma-Aldrich Catalog #10065) ~30 U/mg with 0.4% CaCl₂ and 0.5% NaCl and tested with the protocols described in the above examples. The concentrations tested on this study were as follows:

TABLE 18

| | |
|---|---|
| PEG 400 | 1% |
| CMC | 2.5% |
| Glycerin | 1% |
| PVP | 2% |
| Propylene Glycol | 1% |
| PVA—Polyvinyl alcohol | 4% |
| Dextran 70, Hypromellose | 0.1%, 0.3% |

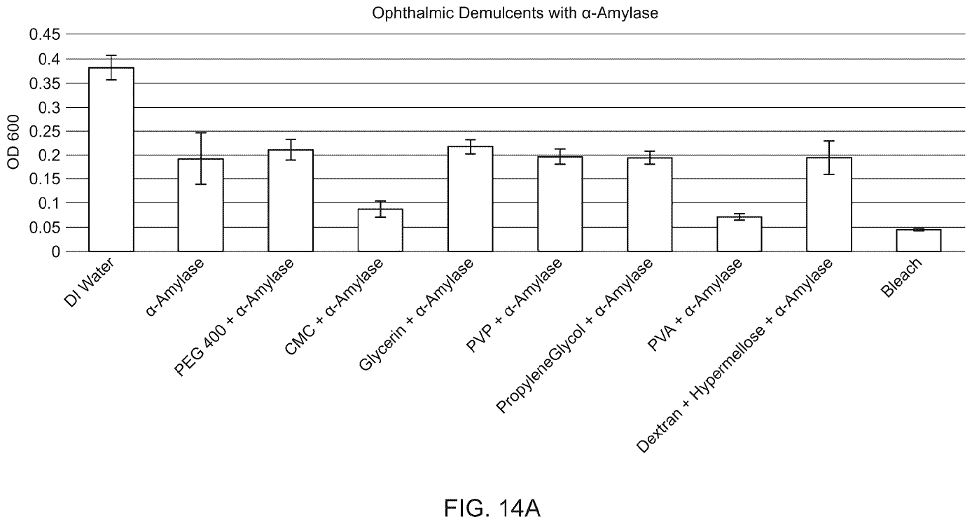

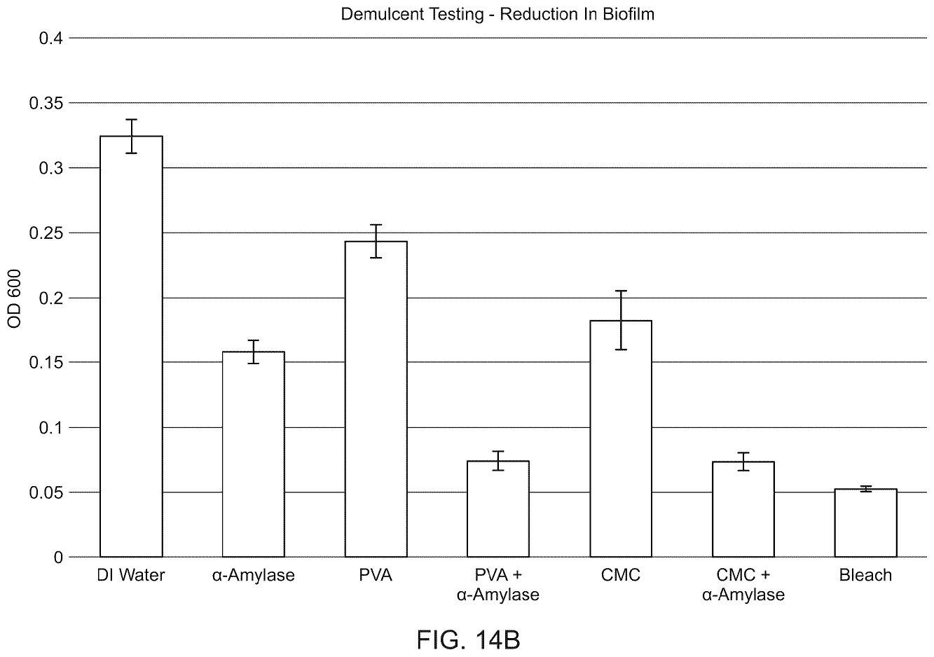

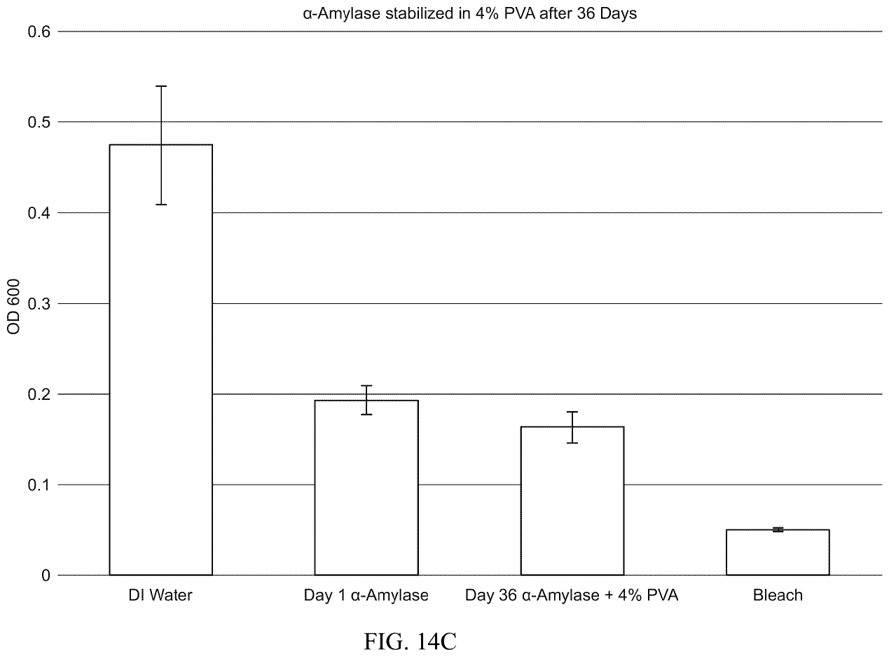

FIG. 14A is a chart illustrating the results of an analysis of the various demulcents tested, including polyethylene glycol 400 (PEG 400) 1%, carboxymethylcellulose sodium (CMC) 2.5%, glycerin 1%, povidone (PVP) 2%, propylene glycol 1%, polyvinyl alcohol (PVA) 4%, dextran 70 0.1 percent+hypromellose 0.3%. FIG. 14B is a chart illustrating the results of an analysis of various demulcents tested, including polyvinyl alcohol (PVA), PVA+α-amylase, carboxymethylcellulose sodium (CMC), and carboxymethylcellulose sodium (CMC)+α-amylase. As shown in FIGS. 14A and 14B, CMC and PVA showed a beneficial effect on the removal of biofilms. Based on these results, it was hypothesized that PVA may help stabilize α-amylase in certain configurations of the formulation.

FIG. 14C is a chart illustrating the results of testing with a-Amylase stabilized in 4% PVA after 36 days. It was found that PVA and CMC independently aid in the removal of biofilms and also work with α-amylase to remove the biofilm. PVA is preferred over CMC for filtration sterilization and lack of susceptibility to bacterial growth. PVA provides a pH of 5.8 which is ideal for enzyme activation. Literature suggests a pH between 5-6 is ideal for α-amylase from Aspergillus oryzae.

PVA solutions are better candidates for sterilization filtration than CMC solutions. The solution of 4% PVA was filtered sterilized using a 2 um sterile syringe filter and kept refrigerated for 36 days before repeating biofilm removal testing. The 4% PVA solution at day 36 performed better than a fresh solution of α-amylase with cofactors. This indicates that PVA can stabilize the α-amylase enzyme for at least 36 days in an aqueous solution refrigerated at 4° C.

### Example 9. Antibiotic Testing

This protocol investigated the efficacy of α-amylase from Aspergillus oryzae combined with commonly prescribed ophthalmic antibiotics to see if this aided in the removal of biofilms. Typically, antibiotics are not believed to penetrate biofilms on their own. Biofilms can also allow bacteria to become antibiotic resistant as bacteria can transfer DNA within the biofilm.

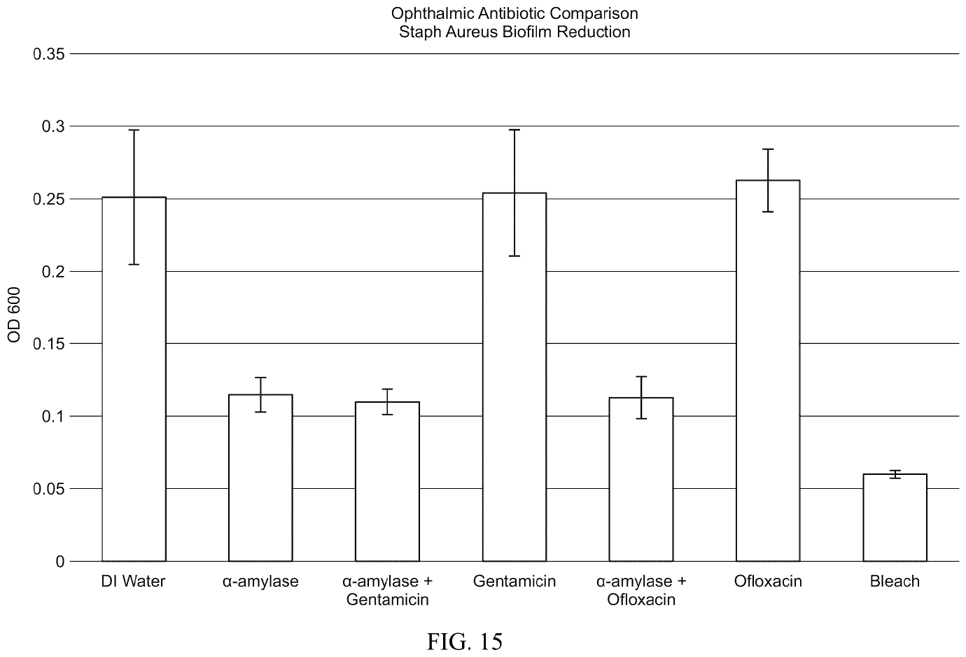

FIG. 15 is a chart illustrating the results of an ophthalmic antibiotic comparison for staph aureus biofilm reduction. 10 mg/mL of α-amylase from Aspergillus oryzae (Sigma-Aldrich Catalog #10065) formulated with 30 U/mg with 0.4% CaCl₂ and 0.56% NaCl as cofactors was used in formulations with gentamicin and ofloxacin. Both antibiotics were also tested on their own.

The results of the study shown in FIG. 15 indicate that the ability for α-amylase from Aspergillus oryzae to remove biofilms is not inhibited or enhanced by topical antibiotics. The combination of ophthalmic antibiotics and α-amylase could aid patients with a chronic infection where bacteria are enclosed in a biofilm.

### Example 10. Stabilizing Sugars

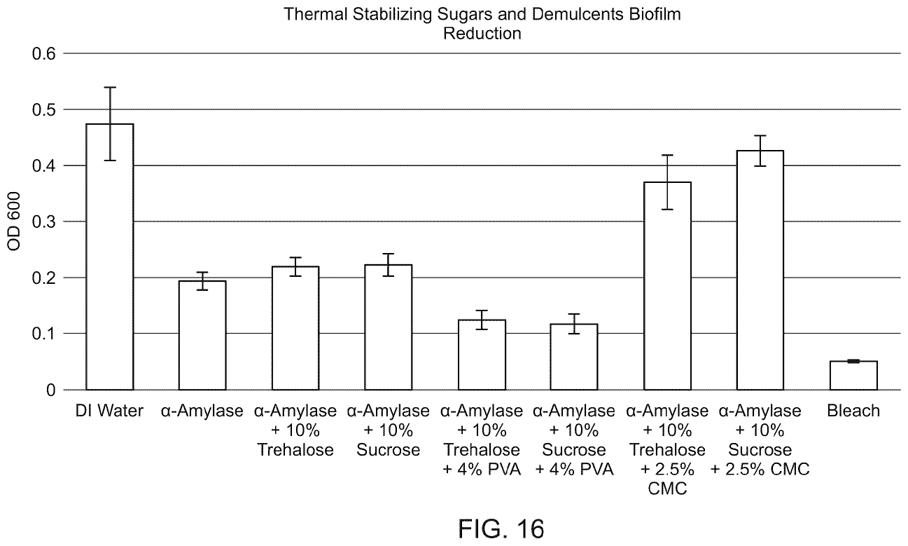

The use of sugars, especially Sucrose and Trehalose have been previously reported to increase the thermal stability of α-amylase from Aspergillus oryzae in aqueous solutions. Trehalose is used in eyedrops for dry eye patients for its bioprotectant properties for epithelial cells of the eye. Solutions of up to 200 mM (6.8%) of Trehalose have been used in commercial eye drops. This experiment tests Sucrose and Trehalose at 10% solutions with and without the demulcents Carboxymethylcellulose and Polyvinyl Alcohol to see if they impact the enzyme's ability to reduce biofilms. This effects of these formulation on biofilm reduction is shown on FIG. 16.

These data indicate that both stabilizing sugars do not have any statistically significant (α=0.05) impact on the enzyme's ability to reduce biofilms. When the stabilizing sugars were combined with Carboxymethylcellulose, the enzyme's ability to reduce biofilms was limited. This indicates that Carboxymethylcellulose is not an ideal demulcent to be combined with the stabilizing sugars Trehalose or Sucrose. The study indicates that the stabilizing sugars combined with PVA still have a strong ability to remove biofilms. As Trehalose is an ideal stabilizing sugar for use around the eye, the combination of PVA and Trehalose is future examined in the buffer studies below. Trehalose was also studied as an independent variable below.

### Trehalose

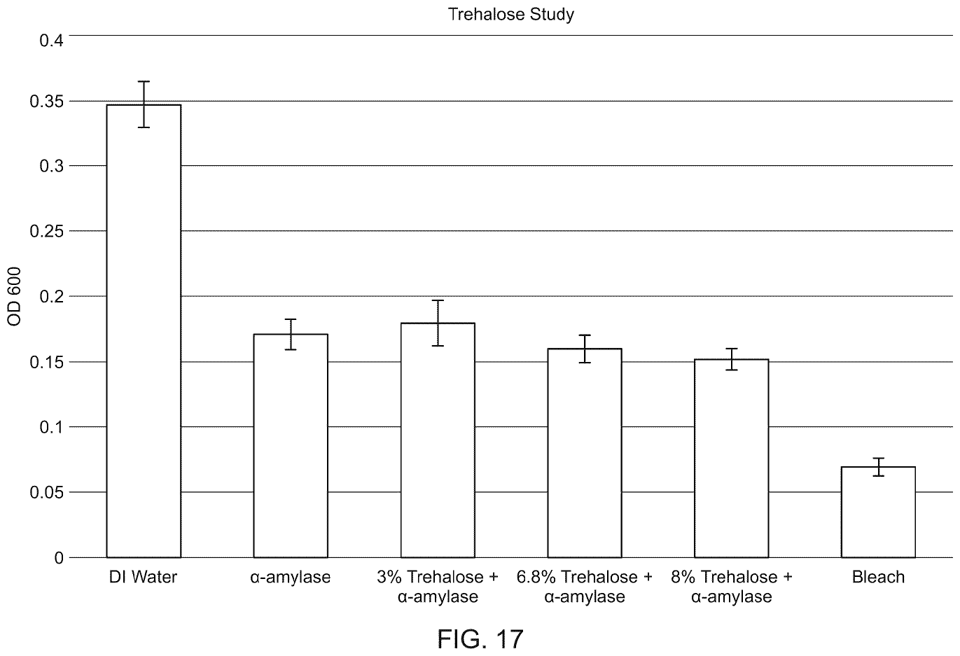

The α-amylase enzyme's ability to break down biofilms was tested with 3%, 6.8%, and 8% Trehalose solutions. There were no significant differences (α=0.05) in the enzyme activity at these concentrations. 6.8% Trehalose was the highest concentration reported in commercially available eye drops and is considered well tolerated. Higher concentrations of Trehalose can be considered as the solution is applied to intact skin and not the eye itself. See FIG. 17.

### Example 11. pH

Literature shows that a-Amylase from Aspergillus oryzae optimal activity is between a pH of 5-6. Tears have a pH of 6.5 to 7.6, normal skin has a pH of 4.7 to 5.75 and oily skin has a pH of 5.7-7. The application of the enzymatic composition will be on the eyelid, not in the eyes. However, due to the proximity to the eyes a pH of 5.8-6 will be used in the final composition to balance enzymatic activity and user comfort. In testing, the disclosure identified the 4% PVA solutions to have a pH of 5.8 and the 2% PVA solutions have a pH of 5.9.

The following buffers are contemplated for pH stabilization:

TABLE 19

| | | | Concentration |
|---|---|---|---|
| Buffer System | Components | pH Range | Range Used |
| Citrate Buffer | Citric Acid (C₆H₈O₇) | 3.0-6.2 | 10-100 mM |
| | Sodium citrate (C₆H₅Na₃O₇) | | |
| Phosphate Buffer | Monosodium Phosphate (H₂NaO₄P) | 5.8-8.0 | 0.1-160 mM |
| | Dibasic sodium phosphate | | |
| | (HNa₂O₄P) | | |
| Tris-HCl Buffer | Tris (hydroxymethyl) aminomethane | 7.2-9.0 | 10-100 mM |
| | (C₄H₁₁NO₃) | | |
| | Hydrochloric acid (HCl) | | |
| Borate Buffer | Boric Acid (BH₃O₃) | 7.6-9.1 | 10-100 mM |
| | Sodium hexaborate | | |

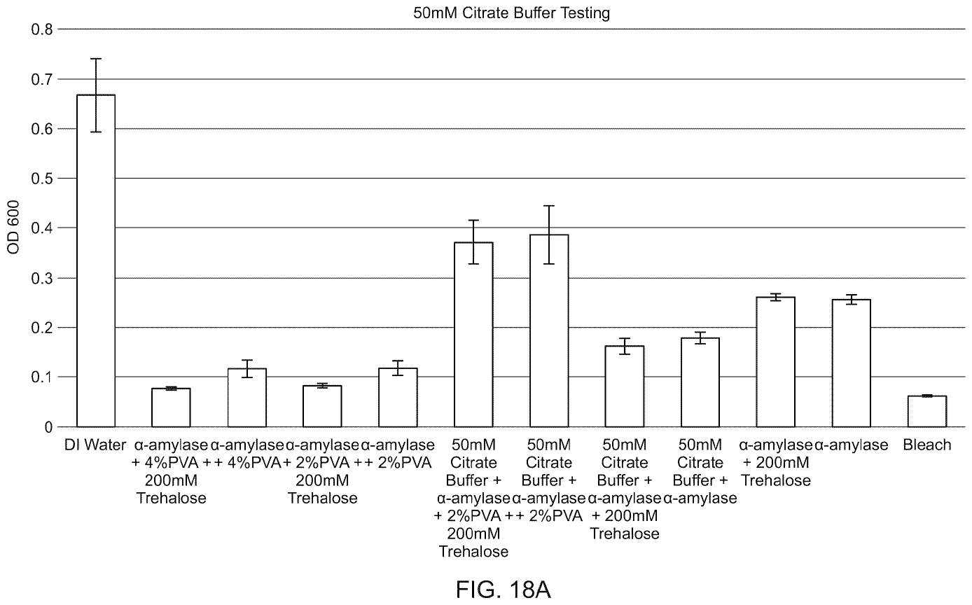

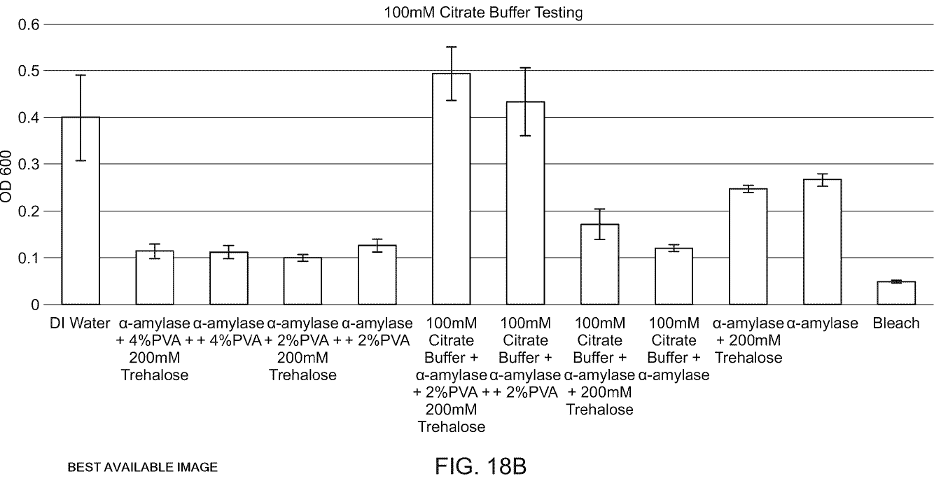

Phosphate buffers and Citrate buffers were further analyzed for use in the composition to maintain a pH of 5.8 as they are ideal candidates in that pH range. When tested with concentrations of 0.4% o CaCl₂, the calcium reacted with phosphate and precipitated. This reaction eliminated phosphate buffers as a candidate if CaCl₂ is used as a cofactor. This suggests that the optimum ophthalmic buffer candidate as the Citrate buffer. Based on literature, solutions were made and tested for their ability to remove biofilms at citrate buffer concentrations of 50 mM and 100 mM. Trehalose used as a thermal stabilizer for the enzyme was added at 200 mM as this was reported well tolerated for use in eyedrops in literature. When the citrate buffer was added directly to a 400 PVA solution the sodium citrate formed a gel and would not dissolve. To test the citrate buffer with PVA, sodium citrate and citric acid were dissolved in DI water and mixed in a 50:50 ratio with the 400 PVA solution to make a 2% o PVA solution for testing. All citrate buffer solutions were brought to a pH of 5.8 using NaOH and HCL. FIGS. 18A and 18B are charts illustrating the results of testing 50 mM-100 mM of citrate buffers with sugar stabilizers and demulcents.

The study indicates that citrate buffers interact with PVA to inhibit the biofilm removal function of the composition at both 50 mM and 100 mM concentrations. Citrate buffers used with NaOH and HCl to bring the composition pH to 5.8 alone enhance the enzyme's performance. See FIG. 18A and FIG. 18B. The best performance of the enzyme was without the citrate buffers with either 2% or 4% PVA with or without Trehalose.

### Example 12. Hyaluronic Acid

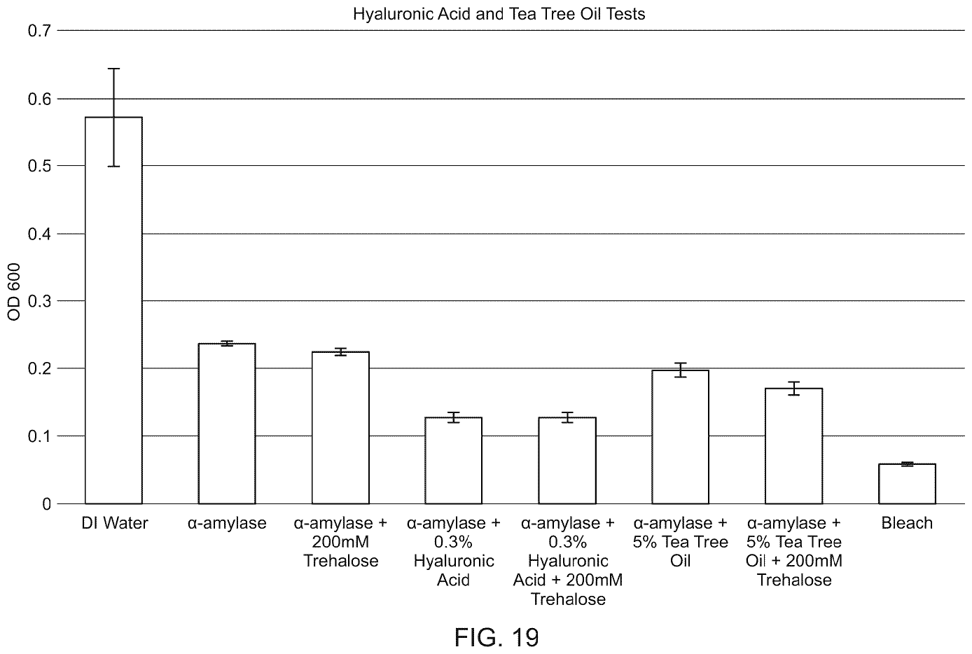

Hyaluronic Acid and Tea Tree Oil are common additives in facial washes and eyelid cleaners. In this study 0.3% Hyaluronic Acid and 5% Tea Tree Oil were added to α-amylase with and without Trehalose to quantify the effect on biofilm removal. FIG. 19 is a chart illustrating the results of testing the effects of hyaluronic acid and tea tree oil on compositions comprising a-amylase. As shown in FIG. 19, 0.3% Hyaluronic acid slightly increased the enzyme's ability to remove biofilm. Tea Tree Oil did not notably enhance or degrade the enzyme's performance. This indicates that Hyaluronic Acid could have a beneficial effect in the composition for biofilm removal. In eye drop formulations, Hyaluronic Acid is often combined with Trehalose for their combined benefit of hydration.

### Example 13. Treatment of a Human Subject with a Composition of the Disclosure

All images are from the same subject at the Watson Dry Eye Center in Raleigh NC at the specified times. The images were reviewed retrospectively to determine if the visual signs of biofilm on the eyelid were removed by a composition of α-amylase from Aspergillus oryzae and 0.01% hypochlorous acid. It should be noted that from May 23, 2018-Nov. 20, 2020 the patient cleaned his eyes with just 0.01% hypochlorous acid in saline two times a day. After this, the patient used the novel composition of α-amylase from Aspergillus oryzae and 0.01% hypochlorous acid. The first composition was made from powdered α-amylase from Aspergillus oryzae and liquid 0.01% hypochlorous acid in saline right before application to the eyelid with a cotton swab. The second composition included Ca²⁺ and had superior performance. Dosage information is written on the timeline below.

### Timeline of Treatment—Prior to Use of a Composition Comprising

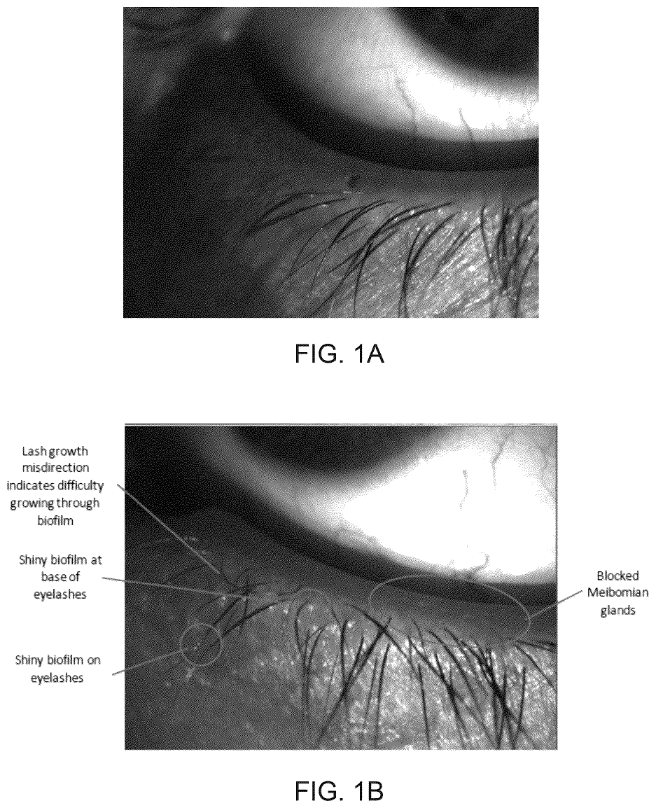

Subject had a first treatment session for treatment of dry eye symptoms and recurrent eye inflammation on May 23, 2018. Prior to this day, subject received a combination of Azasite and oral antibiotics (100 mg doxycycline/day), which did not appear to relieve the symptoms. FIG. 1A and FIG. 1B are photographs depicting two different areas of an eye of the subject afflicted with blocked meibomian glands, shiny biofilm at the base of eyelashes, shine biofilm on eyelashes, and lash growth misdirection taken on May 23, 2018 (time 0). After the photos were taken, the subject was treated with Blephex and Intense Pulse Light Therapy for biofilm removal treating inflammation.

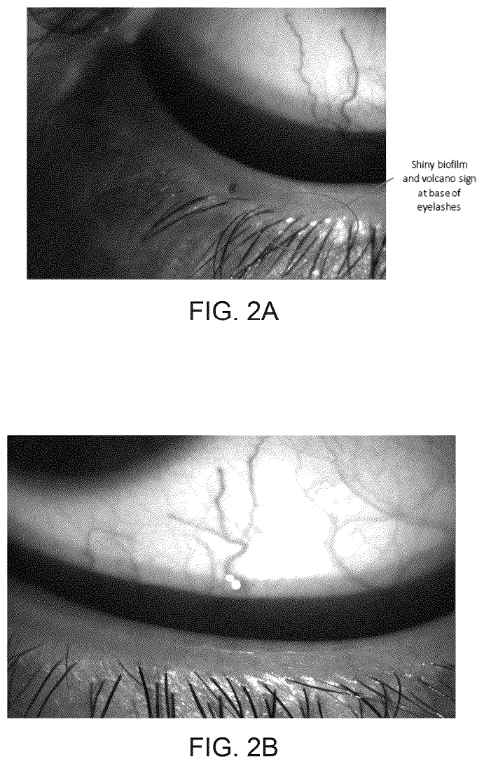

Subject received a subsequent treatment session on Dec. 4, 2018. FIG. 2A and FIG. 2B are photographs depicting the two different areas of the eye of the subject on Dec. 4, 2018, approximately 6 months after the photographs of FIG. 1A and FIG. 1B were taken (time 1). After the photos were taken, the subject received a 1-time treatment of Blephex and Intense Pulse Light Therapy for biofilm removal treating inflammation. Subsequently, subject received daily doses of Avenova (0.01% hypochlorous acid (HOCL)). Minimum improvement to the symptoms was reported on this session.

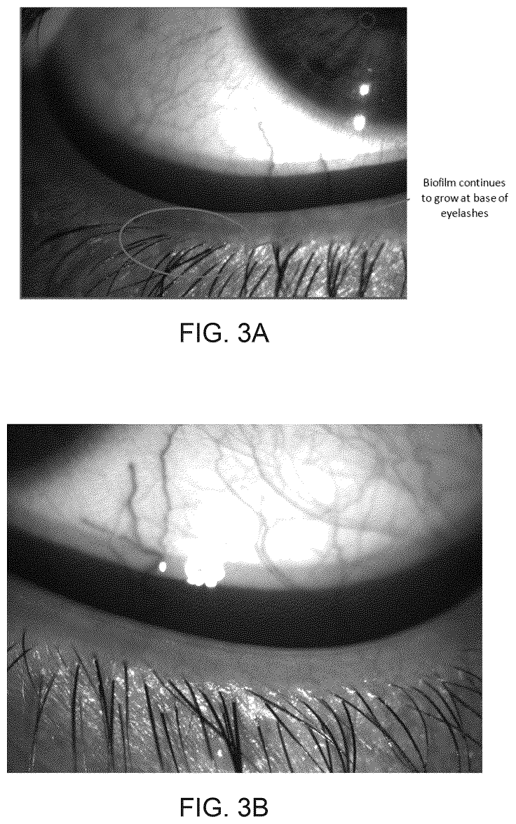

Subject received a subsequent treatment session on June 18 2019 for a daily use of Avenova (0.01% hypochlorous acid (HOCL)). FIG. 3A and FIG. 3B are photographs depicting the two different areas of the eye of the subject approximately 6 months after the photographs of FIG. 2A and FIG. 2B were taken (time 2). After the photos were taken, the subject received a 1-time treatment of Blephex and Intense Pulse Light Therapy for biofilm removal treating inflammation. Subsequently, subject was prescribed daily use of Avenova (0.01% hypochlorous acid (HOCL)). As shown in FIG. 3A, the biofilm continues to grow at the base of the eyelashes.

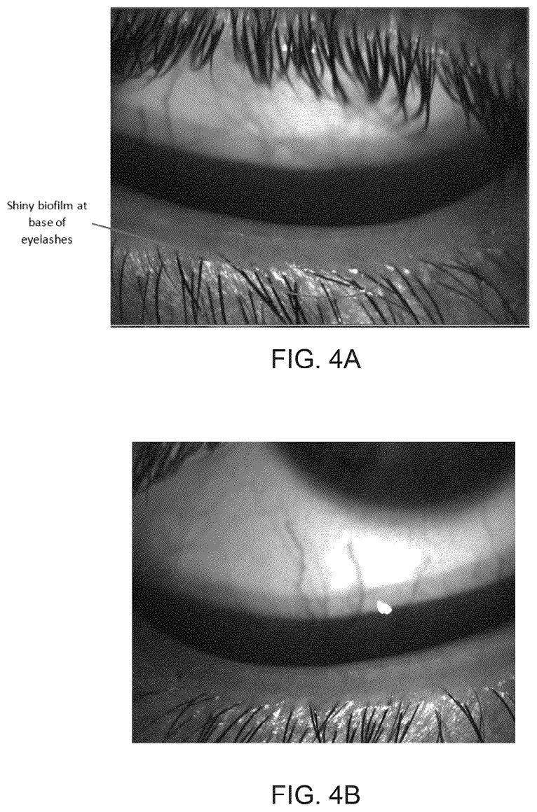

Subject received a subsequent treatment session on October 15 2019 for a daily use of Avenova (0.01% hypochlorous acid (HOCL)). FIG. 4A and FIG. 4B are photographs depicting the two different areas of the eye of the subject approximately 4 months after the photographs of FIG. 3A and FIG. 3B were taken (time 3). After the photos were taken, the subject received a 1-time treatment of Blephex and Intense Pulse Light Therapy for biofilm removal treating inflammation. Subsequently, subject continued to be treated with daily use of Avenova (0.01% hypochlorous acid (HOCL)). As shown in FIGS. 4A and 4B the shine biofilm remains at the eyelashes and inside the eyelid, suggesting that the acid treatment on its own was not sufficient to remove the biofilm.

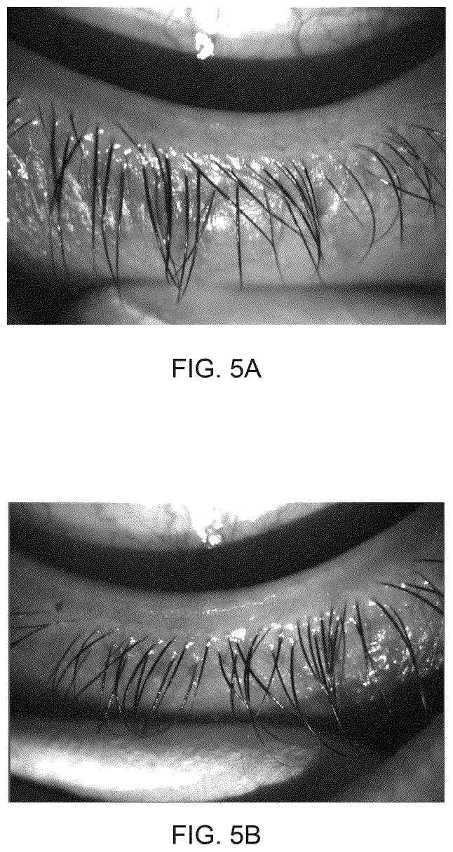

On October 2020, a first composition was prepared with 1 mg/ml α-amylase from Aspergillus oryzae and 0.01% hypochlorous acid. FIG. 5A and FIG. 5B are photographs depicting the two different areas of the eye of the subject approximately on November 2 2020, after a few applications of 1 mg/ml of α-amylase from Aspergillus oryzae and 0.01% hypochlorous acid. This timeframe is approximately about 1-month after the photographs of FIG. 4A and FIG. 4B were taken (time 4). The biofilm was still visibly present after a few low dose applications within the 1-month timeframe for treatment, as shown in FIGS. 5A and 5B.

Subject received daily doses of the composition comprising 1 mg/ml of α-amylase from Aspergillus oryzae and 0.01% hypochlorous acid between time 4 and May 26 2021 (time 5), where the subject was subject to routine daily use of composition FIG. 6A and FIG. 6B are photographs depicting the two different areas of the eye of the subject approximately 6 months after the photographs of FIG. 5A and FIG. 5B were taken. After the photos of FIG. 5A and FIG. 5B were taken, the subject received routine daily use of the composition comprising 1 mg/ml of a-amylase from Aspergillus oryzae and 0.01% hypochlorous acid. As shown in FIGS. 6A and 6B, the biofilm is visibly eliminated, eyelashes are clean of all biofilm, inflammation and redness in eye lid reduced, patient reports major improvements in dry eye symptoms.

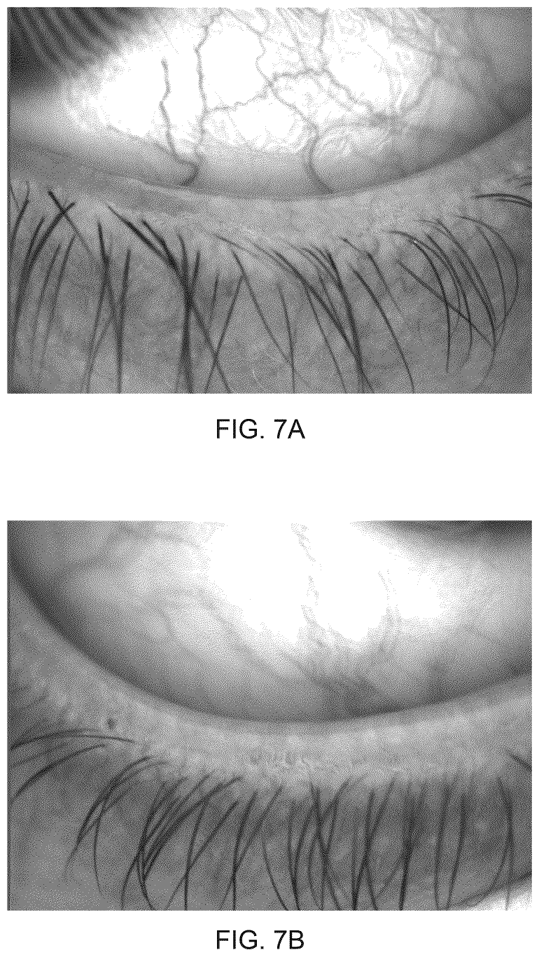

On, Oct. 5, 2021, approximately after 5 months of routine daily use of a composition (time 6) comprising 1 mg/ml of α-amylase from Aspergillus oryzae and 0.01% hypochlorous acid subject reported significantly improved symptoms and new eye images were taken at the at the Watson Dry Eye Center in Raleigh NC. As shown in FIG. 7A and FIG. 7B, the biofilm growth was prevented and the eye lid tissue was no longer inflamed.

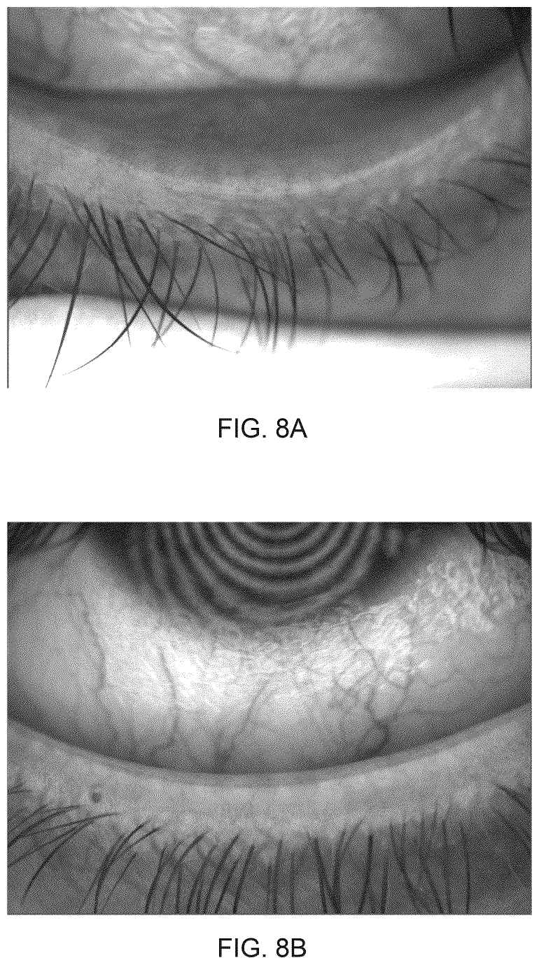

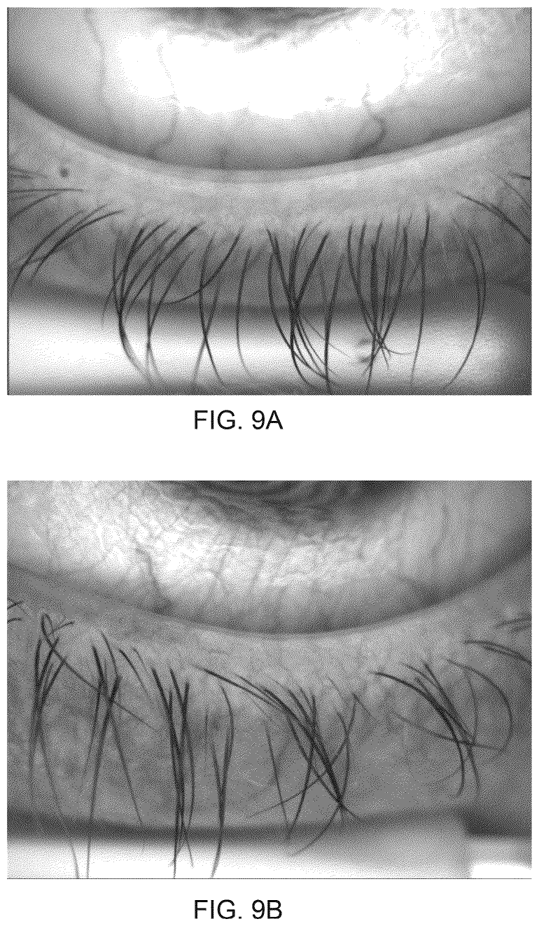

The subject continued to be monitored. On Feb. 3, 2022, approximately 4 months after routine daily use of a composition (time 7) comprising 1 mg/ml of α-amylase from Aspergillus oryzae and 0.01% hypochlorous acid the biofilm growth was prevented on the eyes of the subject, see FIGS. 8A and 8B. Further, no inflammation was observed within that time-frame. Between time 7 and a period of approximately 6 months (August 4 2022), the subject was tested for tolerance of a higher concentration dosage for bi/weekly use, namely 10 mg/ml of α-amylase from Aspergillus oryzae and 0.01% hypochlorous acid. No adverse effects were observed for the higher dose of 10 mg/ml of α-amylase. See FIGS. 9A and 9B.

### Example 14. Treatment of a Human Subject with a Composition of the Disclosure

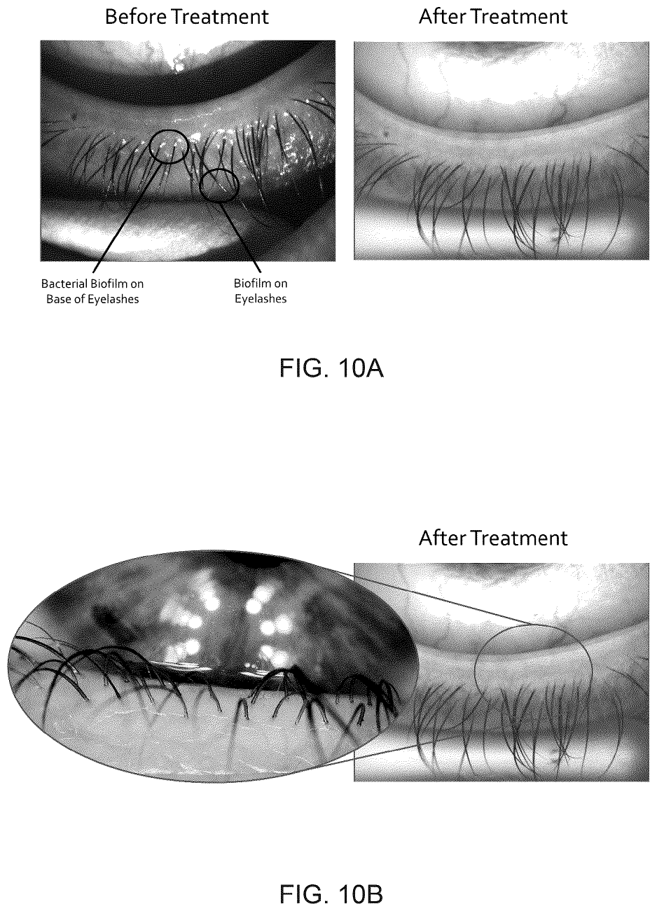

A composition comprising approximately 3% α-amylase, 0.06% sodium chloride, 0.02% hypochlorous acid solution was prepared in ionized water and directly applied bi-weekly to the eyelid margin of a patient who presented with elevated levels of eyelid biofilm. Observations over the course of 6 months indicated a significant reduction in biofilm on the eyelid margin, an increase in tear production, and reduced indications of stress to the ocular surface. A pilot study indicated similar improvements with a composition comprising approximately 3% α-amylase, 1% lysozyme 0.06% sodium chloride, and 0.02% hypochlorous acid solution in ionized water. The composition in the pilot study was prepared fresh prior to each use. Further composition development is investigating the combination of the aforementioned components with preservatives, astringent(s), demulcent(s), emollient(s), hypertonicity agent(s), and vasoconstrictor(s) described throughout the disclosure to improve longer term stability of the composition. FIG. 10A and FIG. 10B are photographs depicting a direct comparison of areas of the eye before and after treatment with a composition that comprises 10 mg/ml of α-amylase in 0.01% hypochlorous acid and Ca²⁺.

### Example 15. Formulation

This protocol outlines the procedure for testing compositions of α-amylase from Aspergillus oryzae with a 0.01% hypochlorous acid for compatibility and ability to remove biofilms from S. Aureus 0.01% hypochlorous acid is intended to prohibit microbial growth, not extend the shelf life on α-amylase. This experiment will also test the effect of hypochlorous acid on the enzyme by using compositions of α-amylase and hypochlorous acid at different time points after the composition is made. The compositions will be tested for its ability to remove biofilms immediately after the composition is made, as well as at 30 minute and 1-hour timepoints. This test will indicate if combining the aqueous hypochlorous acid with a dry α-amylase prior to biofilm application is advantageous.

### Formulation

An off the shelf eyelid cleaner Avenova was be used for this experiment (0.01% Hypochlorous Acid in saline). In the hypochlorous acid solution 0.4% calcium chloride will be added to ensure all solutions tested have calcium ions present.

Prepare a 10 ml solution of 10 mg/ml α-amylase with cofactors.

Add 100 mg of α-amylase from Aspergillus oryzae (Sigma-Aldrich Catalog #10065) to 10 ml sterile DI Water in a flask. Mix until α-amylase is dissolved and evenly distributed.

Dissolve 40 mg of calcium chloride and 56 mg of sodium chloride into the solution.

### 1 Hour Prior to Trial:

Prepare a 5 ml solution of 10 mg/ml α-amylase with hypochlorous acid 0.01%

Add 5 mL of Avenova to flask (hypochlorous acid 0.01%)

Dissolve 20 mg of calcium chloride into the solution.

Add 50 mg of α-amylase from Aspergillus oryzae (Sigma-Aldrich Catalog #10065). Mix until α-amylase is dissolved and evenly distributed.

### 30 Minutes Prior to Trial:

Prepare a 5 ml solution of 10 mg/ml α-amylase with hypochlorous acid 0.01%

Add 5 mL of Avenova to flask (hypochlorous acid 0.01%)

Dissolve 20 mg of calcium chloride into the solution.

Add 50 mg of α-amylase from Aspergillus oryzae (Sigma-Aldrich Catalog #10065). Mix until α-amylase is dissolved and evenly distributed.

### Immediately Before Plating:

Prepare a 5 ml solution of 10 mg/ml α-amylase with hypochlorous acid 0.01%

Add 5 mL of Avenova to flask (hypochlorous acid 0.01%)

Dissolve 20 mg of calcium chloride into the solution.

Add 50 mg of α-amylase from Aspergillus oryzae (Sigma-Aldrich Catalog #10065). Mix until α-amylase is dissolved and evenly distributed.

### Test Compositions

Carefully tilt plate and pipet all liquid from the wells by inserting the pipet tips in the bottom corner of the wells to remove non-adherent cells without disturbing the biofilm grown on the bottom of the well.

Start a timer for 10 minutes when the first row is treated using an 8-channel pipette. Each row should be treated every 30 seconds making sure to gently release the composition in the corner of the well to not disturb the biofilm mechanically.

For Row 1: add 50 μl DI water to row 1.

For Row 2: add 50 μl of 10 mg/ml α-amylase with cofactors to row 2.

For Row 3: add 50 μl of 10 mg/ml α-amylase with cofactors and hypochlorous acid set aside for 30 minutes to row 3.

For Row 4: add 50 μl of 10 mg/ml α-amylase with cofactors and hypochlorous acid set aside for 1 hour to row 4.

For Row 5: combine 5 ml solution of 10 mg/ml α-amylase with hypochlorous acid. Add 50 μl of 10 mg/ml α-amylase with cofactors and hypochlorous acid to row 5.

For Row 6: add 50 μl of 0.018% hypochlorous acid solution to row 7.

For Row 7: add 50 μl of 7.5% bleach (Sodium hypochlorite) solution to row 7.

Incubate plate for the remaining 10 minutes at 37 C.

### Test for Biofilm Reduction

After 10 min all fluid should be removed row by row every 30 seconds starting with Row 1. The fluid must be removed with gentle pipetting, with the plate at an angle, with the pipette in the corner to remove non-adherent cells without disturbing the biofilm grown on the bottom of the well. Add 50 μl of 0.1% crystal violet (CV) to stain adherent cells for all wells. Carefully rinse all wells by submerging the plate in a DI water bath to remove all remaining crystal violet. Dry the well plate and measure the optical density of the biofilms using a plate reader at 600 nm. Once all measurements have been taken the well plate should be transferred to a bleach solution for sanitization and disposed of in a biological waste bin.

### Results

Results showed that there was no statistical significance in biofilm reduction from a-amylase with cofactors and α-amylase with cofactors combined with either hypochlorous acid spray across all timepoints. This indicates that 0.01% hypochlorous acid did not inhibit biofilm removal by the enzyme. While 0.01% hypochlorous acid did slightly reduce the biofilm, when combined with α-amylase and cofactors there was no additive benefit of increased biofilm removal. See FIG. 13.

### Example 16. Enzymatic Facial and Eyelid Cleaners

Various compositions described herein have been developed as enzymatic facial and/or eyelid treatment(s).

The enzymatic facial and eyelid cleanser device utilizes an alpha-amylase enzyme to break down the extracellular matrix of the biofilm that forms on the eyelids. Biofilms are structured communities of microorganisms adhering to surfaces, surrounded by a self-produced extracellular matrix (ECM). The ECM is composed mainly of polysaccharides, proteins, nucleic acids, and lipids. This matrix provides structural stability to the biofilm, protects the bacteria within from external factors, and helps the biofilm adhere to surfaces.

Biofilms are aggregations of microorganisms, such as bacteria, embedded in a self-produced matrix of proteins and polysaccharides on the eyelid. These biofilms can lead to inflammation and infection of the eyelid margin, known as blepharitis, and can also contribute to dry eye disease by disrupting the tear film and causing tear evaporation.

The α-amylase isoform in the composition functions as an enzyme that breaks down the polysaccharides in the biofilm matrix. By doing so, it destabilizes the structure of the biofilm, making it easier to remove from the eyelid. With the ECM destabilized, the biofilm becomes more susceptible to mechanical removal (e.g., by wiping). Additionally, bacteria within the biofilm lose their protective shield, making them more vulnerable to antimicrobial agents or the body's immune response.

In summary the isoform α-amylase from Aspergillus oryzae targets the polysaccharide component of the biofilm's extracellular matrix, breaking it down and facilitating the removal of the biofilm from the eyelids. This can be particularly helpful in managing conditions like blepharitis and dry eye disease, where biofilms contribute to the pathology.

The enzymatic composition can be utilized through different delivery mechanisms such as sterile wipes, sprays, or single or multidose sterile applicators. In some instances, the compositions are cleansers. The compositions can be packaged as a wipe, a spray, a pad, or another suitable applicator infused with an α-amylase powder or solution sourced from Aspergillus oryzae.

### 14.1 Sterile Wipes

A composition of the disclosure is packaged as a sterile wipe(s) pre-saturated with the composition. These wipes are individually packaged or packaged in sets. Such wipes can be further packaged in a container or as part of a kit with other reagents.

Methods of using compositions packaged as sterile wipes: a subject gently closes its eye and uses the sterile wipe to clean the eyelid with, e.g., horizontal strokes, from the inner corner to the outer corner. A subject ensures that the composition is preferentially applied to the base of the eyelashes where biofilms are likely to accumulate.

The wipes are individually packaged for single-use, reducing the risk of contamination. The wipes are portable and convenient for travel or on-the-go use.

### 14.2 Spray

The enzymatic composition can be formulated as a solution within a bottle (e.g., spray or drop).

Methods of using compositions packaged as spray: a spray bottle containing the enzymatic composition can allow for direct application to the eyelid or be sprayed onto a clean cloth or cotton pad for application. A subject may close the eye and lightly spray the solution directly onto the eyelid. Alternatively, a subject may spray the solution onto a clean cloth or cotton pad. If using a cloth or pad, gently wipe the eyelid from the inner corner to the outer corner.

Spray bottles are easy to use and allow for quick application. They are also suitable for multiple uses without direct contact with the eyelid, which reduces contamination risk.

### 14.3 Single or Multidose Sterile Container:

A single or multidose sterile container is used for controlled delivery of the enzymatic solution onto a sterile applicator such as a wipe or sponge and then applied to the eyelid. The applicator could be attached to the sterile container or used separately.

Methods of using compositions packaged in single or multidose sterile container(s): a subject closes the eye and uses the applicator to apply the solution along the eyelid margin and eyelashes. The subject carefully clean the eyelid with horizontal strokes, moving from the inner to the outer corner. The applicator supports targeted application of the solution/gel, which may be beneficial for those who need a more controlled application or have sensitivity issues.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6.

What is claimed is:

1.-29. (canceled)

30. An ophthalmic composition comprising a therapeutically-effective amount of a functionally active α-amylase enzyme for treating a blepharitis or a dry eye condition, a concentration of CaCl₂ for stabilizing the α-amylase enzyme and one or more pharmaceutically-acceptable excipients.

31. The ophthalmic composition of claim 30, wherein the α-amylase enzyme shares 95% sequence homology with an α-Amylase peptide sequence from Aspergillus oryzae.

32. The ophthalmic composition of claim 30, wherein the composition comprises between 1 mg/ml (w/v) of the functionally active α-amylase enzyme to 20 mg/ml (w/v) of the functionally active α-amylase enzyme.

33. The ophthalmic composition of claim 30, wherein the concentration of the CaCl₂ ranges from 0.2% to 0.8% (w/v).

34. The ophthalmic composition of claim 30, wherein the composition further comprises one or more of an ophthalmic astringent, an ophthalmic demulcent, an ophthalmic emollient, an ophthalmic hypertonicity agent, or an ophthalmic vasoconstrictor.

35. The ophthalmic composition of claim 34, wherein the composition further comprises between 0.2% (v/v) to 3.5% (v/v) of carboxymethylcellulose sodium or of hydroxyethyl cellulose.

36. The ophthalmic composition of claim 34, wherein the composition further comprises between 0.2% (v/v) to 3.5% (v/v) of Hypromellose or of methylcellulose.

37. The ophthalmic composition of claim 34, wherein the composition further comprises between 0.01% (v/v) to 1.0% (v/v) of dextran.

38. The ophthalmic composition of claim 34, wherein the composition further comprises between 0.01% (v/v) to 1.5% (v/v) of glycerin.

39. The ophthalmic composition of claim 34, wherein the composition further comprises between 0.05% (v/v) to 1.5% (v/v) of polyethylene glycol 300 or of polyethylene glycol 400.

40. The ophthalmic composition of claim 34, wherein the composition further comprises between 0.2% (v/v) to 1.0% (v/v) of polysorbate.

41. The ophthalmic composition of claim 34, wherein the composition further comprises between 0.2% (v/v) to 2.0% (v/v) of propylene glycol.

42. The ophthalmic composition of claim 34, wherein the composition further comprises between 0.2% (v/v) to 6.0% (v/v) of polyvinyl alcohol.

43. The ophthalmic composition of claim 34, wherein the composition further comprises between 0.1% (v/v) to 4.0% (v/v) of povidone.

44. The ophthalmic composition of claim 34, wherein the composition further comprises between 0.001% (v/v) to 0.1% (v/v) of benzalkonium chloride (BAK).

45. The ophthalmic composition of claim 34, wherein the composition further comprises between 0.0001% (v/v) to 0.01% (v/v) of polyquad (polyquaternium-1).

46. The ophthalmic composition of claim 34, wherein the composition further comprises a stabilized oxychloro complex.

47. The ophthalmic composition of claim 34, wherein the composition further comprises a sodium perborate.

48. The ophthalmic composition of claim 34, wherein the composition further comprises a Edetate disodium and Sorbic acid.

49. The ophthalmic composition of claim 34, wherein the composition further comprises a Borate, sorbitol, propylene glycol, and zinc Ionic buffer.

50. The ophthalmic composition of claim 34, wherein the composition further comprises a citrate buffer.

51. The ophthalmic composition of claim 34, wherein the composition further comprises polyhexanide (polyhexamethylene biguanide).

52. The ophthalmic composition of claim 30, wherein the composition is formulated as a liquid.

53.-67. (canceled)
